SEQUENCE LISTING (34 sequences)

SEQ ID NO: 1 - DNA, 928 bp, Homo sapiens
  CDS (127)...(720)
gcgcgcagcgctggtaccccgttggtccgcgcgttgctgcgttgtgaggggtgtcagctcagtgcatcccaggcagctcttagtgtggagcagtgaactgtgtgtggttccttctacttggggatcatgcagagagcttcacgtctgaagagagagctgcacatgttagccacagagccacccccaggcatcacatgttggcaagataaagaccaaatggatgacctgcgagctcaaatattaggtggagccaacacaccttatgagaaaggtgtttttaagctagaagttatcattcctgagaggtacccatttgaacctcctcagatccgatttctcactccaatttatcatccaaacattgattctgctggaaggatttgtctggatgttctcaaattgccaccaaaaggtgcttggagaccatccctcaacatcgcaactgtgttgacctctattcagctgctcatgtcagaacccaaccctgatgacccgctcatggctgacatatcctcagaatttaaatataataagccagccttcctcaagaatgccagacagtggacagagaagcatgcaagacagaaacaaaaggctgatgaggaagagatgcttgataatctaccagaggctggtgactccagagtacacaactcaacacagaaaaggaaggccagtcagctagtaggcatagaaaagaaatttcatcctgatgtttaggggacttgtcctggttcatcttagttaatgtgttctttgccaaggtgatctaagttgcctaccttgaatttttttttaaatatatttgatgacataatttttgtgtagtttatttatcttgtacatatgtattttgaaatcttttaaacctgaaaaataaatagtcatttaatgttgaaaaaaaaaaaaaaaaaaaaaaaaaaaaaaa

SEQ ID NO: 2 - protein, 197 aa, Homo sapiens
MQRASRLKRELHMLATEPPPGITCWQDKDQMDDLRAQILGGANTPYEKGVFKLEVIIPERYPFEPPQIRFLTPIYHPNIDSAGRICLDVLKLPPKGAWRPSLNIATVLTSIQLLMSEPNPDDPLMADISSEFKYNKPAFLKNARQWTEKHARQKQKADEEEMLDNLPEAGDSRVHNSTQKRKASQLVGIEKKFHPDV

SEQ ID NO: 3 - DNA, 1472 bp, Homo sapiens
  CDS (53)...(1189)
ggccactgagccggggtgcagtggcagcgggagagtacctggcgatggcgatatgagcggtgcgggggtggcggctgggacgcggccccccagctcgccgaccccgggctctcggcgccggcgccagcgcccctctgtgggcgtccagtccttgaggccgcagagcccgcagctcaggcagagcgacccgcagaaacggaacctggacctggagaaaagcctgcagttcctgcagcagcagcactcggagatgctggccaagctccatgaggagatcgagcatctgaagcgggaaaacaaggatctccattacaagctcataatgaatcagacatcacagaagaaagatggcccctcaggaaaccacctttccagggcctctgctcccttgggcgctcgctgggtctgcatcaacggagtgtgggtagagccgggaggacccagccctgccaggctgaaggagggctcctcacggacacacaggccaggaggcaagcgtgggcgtcttgcgggcggtagcgccgacactgtgcgctctcctgcagacagcctctccatgtcaagcttccagtctgtcaagtccatctctaattcaggcaaggccaggccccagcccggctccttcaacaagcaagattcaaaagctgacgtctcccagaaggcggacctggaagaggagcccctacttcacaacagcaagctggacaaagttcctggggtacaagggcaggccagaaaggagaaagcagaggcctctaatgcaggagctgcctgtatggggaacagccagcaccagggcaggcagatgggggcgggggcacaccccccaatgatcctgccccttcccctgcgaaagcccaccacacttaggcagtgcgaagtgctcatccgcgagctgtggaataccaacctcctgcagacccaagagctgcggcacctcaagtccctcctggaagggagccagaggccccaggcagccccggaggaagctagctttcccagggaccaagaagccacgcatttccccaaggtctccaccaagagcctctccaagaaatgcctgagcccacctgtggcggagcgtgccatcctgcccgcactgaagcagaccccgaagaacaactttgccgagaggcagaagaggctgcaggcaatgcagaaacggcgcctgcatcgctcagtgctttgagccaccccaatctggtcagtgccaggcccaccaacctgcagctggagactggctctctatagcatttcctgatacttccgctacttttaggcctggctaaattccaagacagataacactcaagatagataaagtacttgatctccaaactgacaaactgtttattttctagctgttattttgctatttggcatttacataaaagcacacgatgaagcaggtatcgccttacctgttgaaactgaaaataaagcttgtttatttccaaaaaaaaaaaaaaaaa

SEQ ID NO: 4 - protein, 378 aa, Homo sapiens
MSGAGVAAGTRPPSSPTPGSRRRRQRPSVGVQSLRPQSPQLRQSDPQKRNLDLEKSLQFLQQQHSEMLAKLHEEIEHLKRENKDLHYKLIMNQTSQKKDGPSGNHLSRASAPLGARWVCINGVWVEPGGPSPARLKEGSSRTHRPGGKRGRLAGGSADTVRSPADSLSMSSFQSVKSISNSGKARPQPGSFNKQDSKADVSQKADLEEEPLLHNSKLDKVPGVQGQARKEKAEASNAGAACMGNSQHQGRQMGAGAHPPMILPLPLRKPTTLRQCEVLIRELWNTNLLQTQELRHLKSLLEGSQRPQAAPEEASFPRDQEATHFPKVSTKSLSKKCLSPPVAERAILPALKQTPKNNFAERQKRLQAMQKRRLHRSVL

SEQ ID NO: 5 - DNA, 1315 bp, Homo sapiens
  CDS (251)...(1114)
agagaaagtatagccactgcttagacagccagggaaacgtgtgcggggaagtggaggactcaggctctcgtgcgagagcggagttggacgtgcagggccgctggggtcacgcggagctctcccgcctcccctccgcgtgagctctgggatggtccgcgccgggagcgcgcgcgaggcttgaagcgcgggtgaagcgcgcaggtcggagtgacagctgcgctgccggcccggctgcggtcagcaacgcgccatggacgcagagctggcagaggtgcgcgccttgcaagctgagatcgcggccctgcggcgagcgtgtgaggacccaccggcgccctgggaagagaagtcccgagtccaaaaatcttttcaagccatacaccaattcaatttggaaggatggaagtcttcaaaagatctgaaaaatcagcttggacatttagaatcagaactttcatttctaagtacgcttactggcatcaatataagaaatcactccaagcagacagaagacctaacaagcactgagatgacagaaaagagtattagaaaagttctacagagacacagattatcaggaaattgccacatggttacatttcaacttgaatttcagattctggaaattcagaataaggagagattatcttctgctgttactgacctcaacataataatggagcccacagaatgctcagaattaagtgaatttgtgtctagagcagaagagagaaaagatctgttcatgtttttccgaagcctgcatttttttgtggagtggtttgaatatcgtaagcgcacgtttaaacatctcaaggaaaagtacccagatgccgtgtacctctcggaggggccctcctcctgctccatggggatccgcagcgccagccggccagggtttgaattagtcattgtttggaggatacaaatagatgaagatgggaaggtttttccaaagctggatcttctcaccaaagtcccacagcgagccctggagctggacaagaacagagccatagaaactgctcctctcagcttccgaaccctggtaggactgcttggaatcgaagctgctctggaaagcctgataaaatcgctttgtgcagaggagaacaactagttccaaaacagtgaacgtggaggatgaagatgctgcgtggaggaacatgcaattttattcaatataaacatttgctattttctgcttagaaaccacaccctgaagacgtgctgtctatgcagttatggcacattatatggaaactctcatgacatgaaaaataaatacaactagttaagtataaaatgccaaaaaaaa

SEQ ID NO: 6 - protein, 288 aa, Homo sapiens
MDAELAEVRALQAEIAALRRACEDPPAPWEEKSRVQKSFQAIHQFNLEGWKSSKDLKNQLGHLESELSFLSTLTGINIRNHSKQTEDLTSTEMTEKSIRKVLQRHRLSGNCHMVTFQLEFQILEIQNKERLSSAVTDLNIIMEPTECSELSEFVSRAEERKDLFMFFRSLHFFVEWFEYRKRTFKHLKEKYPDAVYLSEGPSSCSMGIRSASRPGFELVIVWRIQIDEDGKVFPKLDLLTKVPQRALELDKNRAIETAPLSFRTLVGLLGIEAALESLIKSLCAEENN

SEQ ID NO: 7 - DNA, 20 bp, Artificial Sequence
  Artificially synthesized primer sequence for RT-PCR
cgaccactttgtcaagctca

SEQ ID NO: 8 - DNA, 23 bp, Artificial Sequence
  Artificially synthesized primer sequence for RT-PCR
ggttgagcacagggtactttatt

SEQ ID NO: 9 - DNA, 23 bp, Artificial Sequence
  Artificially synthesized primer sequence for RT-PCR
caaatattaggtggagccaacac

SEQ ID NO: 10 - DNA, 23 bp, Artificial Sequence
  Artificially synthesized primer sequence for RT-PCR
tagatcaccttggcaaagaacac

SEQ ID NO: 11 - DNA, 20 bp, Artificial Sequence
  Artificially synthesized primer sequence for RT-PCR
acctcaagtccctcctggaa

SEQ ID NO: 12 - DNA, 23 bp, Artificial Sequence
  Artificially synthesized primer sequence for RT-PCR
tcagtttcaacaggtaaggcgat

SEQ ID NO: 13 - DNA, 23 bp, Artificial Sequence
  Artificially synthesized primer sequence for RT-PCR
agagccatagaaactgctcctct

SEQ ID NO: 14 - DNA, 23 bp, Artificial Sequence
  Artificially synthesized primer sequence for RT-PCR
cataactgcatagacagcacgtc

SEQ ID NO: 15 - DNA, 20 bp, Artificial Sequence
  Artificially synthesized primer sequence for RT-PCR
gggaagagaagtcccgagtc

SEQ ID NO: 16 - DNA, 24 bp, Artificial Sequence
  Artificially synthesized primer sequence for RT-PCR
tccttattctgaatttccagaatc

SEQ ID NO: 17 - DNA, 30 bp, Artificial Sequence
  Artificially synthesized primer sequence for 5′ RACE
caagcagtcctaccagggttcggaagctga

SEQ ID NO: 18 - DNA, 30 bp, Artificial Sequence
  Artificially synthesized primer sequence for nested PCR
ccagggttcggaagctgagaggagcagttt

SEQ ID NO: 19 - DNA, 30 bp, Artificial Sequence
  Artificially synthesized primer sequence for PCR
ccggaattcatgcagagagcttcacgtctg

SEQ ID NO: 20 - DNA, 33 bp, Artificial Sequence
  Artificially synthesized primer sequence for PCR
ccgctcgagaacatcaggatgaaatttcttttc

SEQ ID NO: 21 - DNA, 30 bp, Artificial Sequence
  Artificially synthesized primer sequence for PCR
ccggaattcatgagcggtgcgggggtggcg

SEQ ID NO: 22 - DNA, 30 bp, Artificial Sequence
  Artificially synthesized primer sequence for PCR
ccgctcgagaagcactgagcgatgcaggcg

SEQ ID NO: 23 - DNA, 35 bp, Artificial Sequence
  Artificially synthesized primer sequence for PCR
ccggaattcatggacgcagagctggcagaggtgcg

SEQ ID NO: 24 - DNA, 30 bp, Artificial Sequence
  Artificially synthesized primer sequence for PCR
ccgctcgaggttgttctcctctgcacaaag

SEQ ID NO: 25 - DNA, 51 bp, Artificial Sequence
  Artificially synthesized oligonucleotide for siRNA
caccgaagcagcacgacttcttcttcaagagagaagaagtcgtgctgcttc

SEQ ID NO: 26 - DNA, 51 bp, Artificial Sequence
  Artificially synthesized oligonucleotide for siRNA
aaaagaagcagcacgacttcttctctcttgaagaagaagtcgtgctgcttc

SEQ ID NO: 27 - DNA, 18 bp, Artificial Sequence
  Target sequence for siRNA
gaagcagcacgacttctt

SEQ ID NO: 28 - DNA, 19 bp, Artificial Sequence
  Target sequence for siRNA
catcgcaactgtgttgacc

SEQ ID NO: 29 - DNA, 19 bp, Artificial Sequence
  Target sequence for siRNA
tgccagacagtggacagag

SEQ ID NO: 30 - DNA, 19 bp, Artificial Sequence
  Target sequence for siRNA
gcctgcagttcctgcagca

SEQ ID NO: 31 - DNA, 19 bp, Artificial Sequence
  Target sequence for siRNA
gcttccagtctgtcaagtc

SEQ ID NO: 32 - DNA, 19 bp, Artificial Sequence
  Target sequence for siRNA
agcagaggcctctaatgca

SEQ ID NO: 33 - DNA, 19 bp, Artificial Sequence
  Target sequence for siRNA
actgctcctctcagcttcc

SEQ ID NO: 34 - DNA, 19 bp, Artificial Sequence
  Target sequence for siRNA
gtacgcttactggcatcaa

(19) United States
(12) Patent Application Publication
(10) Pub. No.: US 2007/0202109 A1
(43) Pub. Date: Aug. 30, 2007

(54) Method Of Diagnosing Breast Cancer

(75) Inventors: Yusuke Nakamura, Yokohama-shi (JP); Toyomasa Katagiri, Shinagawa-ku (JP); Shuichi Nakatsuru, Saitama-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Oncotherapy Science, Inc., Kawasaki-shi, Kanagawa (JP)

(21) Appl. No.: 10/573,522

(22) PCT Filed: Aug. 10, 2004
(86) PCT No.: PCT/JP04/11741

§ 371 Date: Jul. 25, 2006

Related U.S. Application Data
(60) Provisional application No. 60/505,571, filed on Sep. 24, 2003.

(51) Int. Cl. A61K 48/00; C12Q 1/68; G01N 33/574; A61K 39/395

(52) U.S. Cl. 424/155.1; 435/6; 435/7.23; 514/44

(57) ABSTRACT

Compositions and objective methods for detecting, diagnosing, and treating breast cancer (BRC) are described herein. In particular, the present invention describes three BRC-associated genes, referred to herein as A5657, B9769, and C7965, up-regulated in BRC cells as compared to normal cells. In one embodiment, the diagnostic method involves determining the expression level of a BRC-associated gene that discriminates between BRC cells and normal cells; in an alternate embodiment, the diagnostic method involves determining the expression level of a BRC-associated gene that discriminates among BRC cells, between DCIS cells and IDC cells. The present invention further provides methods of screening for therapeutic agents useful in the treatment of breast cancer, methods of treating breast cancer and methods for vaccinating a subject against breast cancer.

This application claims the benefit of U.S. Provisional Application Ser. No. 60/505,571 filed Sep. 24, 2003, the contents of which are hereby incorporated by reference in its entirety.

### TECHNICAL FIELD

The present invention relates to methods of diagnosing breast cancer.

### BACKGROUND OF THE INVENTION

Breast cancer is a complex disease characterized by the numerous genetic and epigenetic changes in a large number of genes (Katherine N. N., Richard W. and Barbara L. W. Breast cancer genetics: What we know and what we need. Nat Med, 7(5): 552-556, 2001.). Little is known about whether these abnormalities are the cause of breast tumorigenesis, although it has been reported to occur by a multi-step process which can be broadly equated to transformation of normal cells, including the stages of atypical ductal hyperplasia, ductal carcinoma in situ (DCIS) and invasive ductal carcinoma (IDC). Although the stages of mammary carcinogenesis are similar to those in other tissues, the precise molecular mechanisms driving breast cancer remain unknown. In any event, it is readily apparent that molecular factors leading to development of primary breast cancer, its progression, and its metastasis would be valuable targets for the development of better tools for early diagnosis, treatment, and prevention of this disease.

There is evidence that only a portion of premalignant lesions are committed to progression to invasive cancer while the other lesions undergo spontaneous regression. This explanation of molecular participation, which leads to development of primary breast cancer, its progression, and its formation of metastases, is the main focus for new strategies targeted at prevention and treatment.

Gene-expression profiles generated by cDNA microarray analysis can provide considerably more detail about the nature of individual cancers than traditional histopathological methods are able to supply. The promise of such information lies in its potential for improving clinical strategies for treating neoplastic diseases and developing novel drugs (Petricoin, E. F., 3rd, Hackett, J. L., Lesko, L. J., Puri, R. K., Gutman, S. I., Chumakov, K., Woodcock, J., Feigal, D. W., Jr., Zoon, K. C., and Sistare, F. D. Medical applications of microarray technologies: a regulatory science perspective. Nat Genet, 32 Suppl: 474-479, 2002; Johannes B., Esther Z. and Axel U. Molecular targets for breast cancer therapy and prevention. Nat Med, 7 (5): 548-552, 2001.). With this goal in mind, the present inventors have analyzed the expression profiles of tumor or tumors from various tissues by cDNA microarrays (Okabe, H. et al., Genome-wide analysis of gene expression in human hepatocellular carcinomas using cDNA microarray: identification of genes involved in viral carcinogenesis and tumor progression. Cancer Res, 61: 2129-2137, 2001.; Hasegawa, S. et al., Genome-wide analysis of gene expression in intestinal-type gastric cancers using a complementary DNA microarray representing 23,040 genes. Cancer Res, 62: 7012-7017, 2002.; Kaneta, Y. et al., and Ohno, R Prediction of Sensitivity to STI571 among Chronic Myeloid Leukemia Patients by Genome-wide cDNA Microarray Analysis. Jpn J Cancer Res, 93: 849-856,2002.; Kaneta, Y. et al., Genome-wide analysis of gene-expression profiles in chronic myeloid leukemia cells using a cDNA microarray. Int J Oncol, 23: 681-691, 2003.; Kitahara, O. et al., Alterations of gene expression during colorectal carcinogenesis revealed by cDNA microarrays after laser-capture microdissection of tumor tissues and normal epithelia. Cancer Res, 61: 3544-3549, 2001.; Lin, Y. et al. Molecular diagnosis of colorectal tumors by expression profiles of 50 genes expressed differentially in adenomas and carcinomas. Oncogene, 21: 4120-4128, 2002.; Nagayama, S. et al., Genome-wide analysis of gene expression in synovial sarcomas using a cDNA microarray. Cancer Res, 62: 5859-5866, 2002.; Okutsu, J. et al., Prediction of chemosensitivity for patients with acute myeloid leukemia, according to expression levels of 28 genes selected by genome-wide complementary DNA microarray analysis. Mol Cancer Ther, 1: 1035-1042, 2002.; Kikuchi, T. et al., Expression profiles of non-small cell lung cancers on cDNA microarrays: identification of genes for prediction of lymph-node metastasis and sensitivity to anti-cancer drugs. Oncogene, 22: 2192-2205, 2003.).

Recent examination into the expression levels of thousands of genes through the use of cDNA microarrays have resulted in the discovery of distinct patterns in different types of breast cancer (Sgroi, D. C. et al., In vivo gene expression profile analysis of human breast cancer progression. Cancer Res, 59: 5656-5661, 1999.; Sorlie, T. et al., Gene expression patterns of breast carcinomas distinguish tumor subclasses with clinical implications. Proc Natl Acad Sci USA, 98: 10869-10874, 2001.; Kauraniemi, P. et al., New amplified and highly expressed genes discovered in the ERBB2 amplicon in breast cancer by cDNA microarrays. Cancer Res, 61: 8235-8240, 2001.; Gruvberger, S. et al., S. Estrogen receptor status in breast cancer is associated with remarkably distinct gene expression patterns. Cancer Res, 61: 5979-5984, 2001.; Dressman, M. et al., Gene expression profiling detects gene amplification and differentiates tumor types in breast cancer. Cancer Res, 63: 2194-2199, 2003.).

Studies into gene-expression profiles in breast cancers have resulted in the identification of genes that may serve as candidates for diagnostic markers or prognosis profiles. However, these data, derived primarily from tumor masses, cannot adequately reflect expressional changes during breast carcinogenesis, because breast cancer cells exist as a solid mass with a highly inflammatory reaction and containing various cellular components. Therefore, previously published microarray data is likely to reflect heterogenous profiles.

Studies designed to reveal mechanisms of carcinogenesis have already facilitated the identification of molecular targets for certain anti-tumor agents. For example, inhibitors of farnesyltransferase (FTIs) which were originally developed to inhibit the growth-signaling pathway related to Ras, whose activation depends on post-translational farnesylation, have been shown to be effective in treating Ras-dependent tumors in animal models (He et al., Cell 99:335-45 (1999)). Similarly, clinical trials on humans using a combination of anti-cancer drugs and the anti-HER2 monoclonal antibody, trastuzumab, with the aim of antagonizing the proto-oncogene receptor HER2/neu have achieved improved clinical response and overall survival of breast-cancer patients (Lin et al., Cancer Res 61:6345-9 (2001)). Finally, a tyrosine kinase inhibitor, STI-571, which selectively inactivates bcr-abl fusion proteins, has been developed to treat chronic myelogenous leukemias wherein constitutive activation of bcr-abl tyrosine kinase plays a crucial role in the transformation of leukocytes. Agents of these kinds are designed to suppress oncogenic activity of specific gene products (Fujita et al., Cancer Res 61:7722-6 (2001)). Accordingly, it is apparent that gene products commonly up-regulated in cancerous cells may serve as potential targets for developing novel anti-cancer agents.

It has been further demonstrated that CD8+ cytotoxic T lymphocytes (CTLs) recognize epitope peptides derived from tumor-associated antigens (TAAs) presented on the MHC Class I molecule, and lyse tumor cells. Since the discovery of the MAGE family as the first example of TAAs, many other TAAs have been discovered using immunological approaches (Boon, Int J Cancer 54: 177-80 (1993); Boon and van der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994)). Some of the newly discovered TAAs are currently undergoing clinical development as targets of immunotherapy. TAAs discovered so far include MAGE (van der Bruggen et al., Science 254: 1643-7 (1991)), gp100 (Kawakami et al., J Exp Med 180: 347-52 (1994)), SART (Shichijo et al., J Exp Med 187: 277-88 (1998)), and NY-ESO-1 (Chen et al., Proc Natl Acad Sci USA 94: 1914-8 (1997)). On the other hand, gene products demonstrated to be specifically over-expressed in tumor cells have been shown to be recognized as targets inducing cellular immune responses. Such gene products include p53 (Umano et al., Brit J Cancer 84: 1052-7 (2001)), HER2/neu (Tanaka et al., Brit J Cancer 84: 94-9 (2001)), CEA (Nukaya et al., Int J Cancer 80: 92-7 (1999)), and so on.

In spite of significant progress in basic and clinical research concerning TAAs (Rosenberg et al., Nature Med 4: 321-7 (1998); Mukherji et al., Proc Natl Acad Sci USA 92: 8078-82 (1995); Hu et al., Cancer Res 56: 2479-83 (1996)), only limited number of candidate TAAs for the treatment of adenocarcinomaas, including colorectal cancer, are currently available. TAAs abundantly expressed in cancer cells yet whose expression is restricted to cancer cells would be promising candidates as immunotherapeutic targets. Further, identification of new TAAs inducing potent and specific anti-tumor immune responses is expected to encourage clinical use of peptide vaccination strategies for various types of cancer (Boon and can der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994); Shichijo et al., J Exp Med 187: 277-88 (1998); Chen et al., Proc Natl Acad Sci USA 94: 1914-8 (1997); Harris, J Natl Cancer Inst 88: 1442-5 (1996); Butterfield et al., Cancer Res 59: 3134-42 (1999); Vissers et al., Cancer Res 59: 5554-9 (1999); van der Burg et al., J Immunol 156: 3308-14 (1996); Tanaka et al., Cancer Res 57: 4465-8 (1997); Fujie et al., Int J Cancer 80: 169-72 (1999); Kikuchi et al., Int J Cancer 81: 459-66 (1999); Oiso et al., Int J Cancer 81: 387-94 (1999)).

It has been repeatedly reported that peptide-stimulated peripheral blood mononuclear cells (PBMCs) from certain healthy donors produce significant levels of IFN-α in response to the peptide, but rarely exert cytotoxicity against tumor cells in an HLA-A24 or -A0201 restricted manner in ⁵¹Cr-release assays (Kawano et al., Cancer Res 60: 3550-8 (2000); Nishizaka et al., Cancer Res 60: 4830-7 (2000); Tamura et al., Jpn J Cancer Res 92: 762-7 (2001)). However, both of HLA-A24 and HLA-A0201 are popular HLA alleles in the Japanese, as well as the Caucasian populations (Date et al., Tissue Antigens 47: 93-101 (1996); Kondo et al., J Immunol 155: 4307-12 (1995); Kubo et al., J Immunol 152: 3913-24 (1994); Imanishi et al., Proceeding of the eleventh International Histocompatibility Workshop and Conference Oxford University Press, Oxford, 1065 (1992); Williams et al., Tissue Antigen 49: 129 (1997)). Thus, antigenic peptides of carcinomas presented by these HLAs may be especially useful for the treatment of carcinomas among Japanese and Caucasians. Further, it is known that the induction of low-affinity CTL in vitro usually results from the use of peptide at a high concentration, generating a high level of specific peptide/MHC complexes on antigen presenting cells (APCs), which will effectively activate these CTL (Alexander-Miller et al., Proc Natl Acad Sci USA 93: 4102-7 (1996)).

Accordingly, in an effort to understand the carcinogenic mechanisms associated with cancer and identify potential targets for developing novel anti-cancer agents, large scale analyses of gene expression patterns in purified populations of breast cancer cells were performed using a cDNA microarray representing 23,040 genes. More particularly, using a combination of cDNA microarray and laser beam microdissection, precise genome-wide expression profiles of 81 breast tumors were examined, including 12 ductal carcinomas in situ (DCIS) and 69 invasive ductal carcinomas (IDC). Among the up-regulated genes identified, the following three genes were selected for being significantly over-expressed in breast cancer cells: A5657 (previously referred to as AF161499); B9769 (previously referred to as AA156269); and C7965 (previously referred to as AW977394). The findings discussed in detail herein suggest that these genes play key roles in tumor cell growth proliferation, and are therefore promising targets for development of anti-cancer drugs.

The present invention relates to the discovery of the following three genes whose expression was significantly up-regulated in 38 of 49, 30 of 73 and 28 of 49 breast cancer cases assayed, respectively: A5657 (SEQ ID NO: 1), encoding an HSPC150 protein (SEQ ID NO: 2) similar to ubiquitin-conjugating enzyme; B9769 (SEQ ID NO: 3), encoding hypothetical protein BC016861 (SEQ ID NO: 4); and C7965 (SEQ ID NO: 5), designated as LOC90557 (SEQ ID NO: 6). Subsequent semi-quantitative RT-PCR and Northern blot analysis confirmed that A5657, B9769 and C7965 were significantly over-expressed in clinical breast cancer samples and breast cancer cell lines as compared to normal human tissues, including breast ductal cells and normal breast. In particular, B9769 was highly expressed in ERα positive breast cancer cell lines. Immunocytochemical staining showed that exogenous A5657, B9769 and C7965 localized to the cytoplasmic and/or nucleus apparatus, the cytoplasmic, and the cytoplasmic apparatus, respectively, in breast cancer cell line, T47D cells. In particular, exogenous B9769 was observed in the intermediate filament network in COS7 and T47D cells. Furthermore, the A5657 protein was shown to interact with ubiquitin by immunoprecipitation binding assay, which suggests that the A5657 protein potentially has E2 ubiquitin-enzyme activity. Treatment of breast cancer cells with small interfering RNAs (siRNAs) effectively inhibited expression of AS657, B9769 and C7965 and suppressed cell/tumor growth of breast cancer, respectively. In addition, it was discovered that all three genes, when transiently over-expressed in NIH3T3 cells, dramatically promoted cell proliferation in MTT assay, which suggests that they play key roles in cell growth proliferation. These findings suggest that over-expression of A5657, B9769 and C7965 may be involved in breast tumorigenesis and may be promising strategies for specific treatment for breast cancer patients.

Accordingly, the present invention involves the discovery of that the expression of these three genes significantly correlates with breast cancer (BRC). These genes, differentially expressed in breast cancer, are collectively referred to herein as “BRC nucleic acids” or “BRC polynucleotides” and the corresponding encoded polypeptides are referred to as “BRC polypeptides” or “BRC proteins.”

Accordingly, the present invention provides a method of diagnosing or determining a predisposition to breast cancer in a subject by determining an expression level of a BRC-associated gene in a patient-derived biological sample, such as tissue sample. The term “BRC-associated gene” refers to a gene that is characterized by an expression level which differs in a BRC cell as compared to a normal cell. A normal cell is one obtained from breast tissue. In the context of the present invention, a BRC-associated gene is one or more genes selected from the group consisting of A5657 (SEQ ID NO: 1), B9769 (SEQ ID NO: 3) and C7965 (SEQ ID NO: 5). An alteration, e.g., an increase in the level of expression of a BRC-associated gene as compared to a normal control level of the gene, indicates that the subject suffers from or is at risk of developing BRC.

In the context of the present invention, the phrase “control level” refers to a protein expression level detected in a control sample and includes both a normal control level and an breast cancer control level. A control level can be a single expression pattern derived from a single reference population or from a plurality of expression patterns. For example, the control level can be a database of expression patterns from previously tested cells. A “normal control level” refers to a level of gene expression detected in a normal, healthy individual or in a population of individuals known not to be suffering from breast cancer. A normal individual is one with no clinical symptoms of breast cancer. On the other hand, a “BRC control level” refers to an expression profile of BRC-associated genes found in a population suffering from BRC.

An increase in the expression level of one or more BRC-associated genes, selected from the group consisting of A5657, B9769 and C7965, detected in a test sample as compared to a normal control level indicates that the subject (from which the sample was obtained) suffers from or is at risk of developing BRC.

Alternatively, expression of a panel of two or more BRC-associated genes in a sample can be compared to a BRC control level of the same panel of genes. A similarity between a sample expression and BRC control expression indicates that the subject (from which the sample was obtained) suffers from or is at risk of developing BRC.

According to the present invention, gene expression is deemed “altered” when it is increased by at least 10%, at least 25%, at least 50% or more as compared to a control level. Alternately, gene expression may be deemed to be altered if it is increased at least 1, at least 2, at least 5 or more fold as compared to a control level. Gene expression may be determined by detecting hybridization, e.g., on an array, of a BRC-associated gene probe to a gene transcript of the patient-derived tissue sample.

In the context of the present invention, the patient-derived tissue sample can be any tissue obtained from a test subject, e.g., a patient known to or suspected of having BRC. For example, the tissue may contains an epithelial cell. More particularly, the tissue may be an epithelial cell from a breast ductal carcinoma.

The present invention also provides a BRC reference expression profile, comprising a gene expression level of two or more of BRC-associated genes selected from the group consisting of A5657, B9769, and C7965.

The present invention further provides methods of identifying an agent that inhibits the expression or activity of a BRC-associated gene, e.g. BRC-associated genes selected from the group consisting of A5657, B9769 and C7965, by contacting a test cell expressing a BRC-associated gene with a test compound and determining the expression level or activity of the BRC-associated gene. The test cell may be an epithelial cell, such as an epithelial cell obtained from a breast carcinoma A decrease in the expression level or activity a BRC-associated gene or its gene product as compared to a normal control level or activity of the gene or gene product indicates that the test agent is an inhibitor of the BRC-associated gene and may be used to reduce a symptom of BRC, e.g. the expression of a BRC-associated gene selected from the group consisting of A5657, B9769 and C7965.

The present invention also provides a kit comprising a detection reagent which binds to one or more BRC nucleic acids or BRC polypeptides. Also provided is an array of nucleic acids that binds to one or more BRC nucleic acids.

Therapeutic methods of the present invention include a method of treating or preventing BRC in a subject including the step of by administering to the subject an antisense composition. In the context of the present invention, the antisense composition reduces the expression of the specific target gene. For example, the antisense composition may contain a nucleotide which is complementary to a BRC-associated gene sequence selected from the group consisting of BRC-associated genes A5657, B9769 and C7965. Alternatively, the present method may include the steps of administering to a subject a short interfering RNA (siRNA) composition. In the context of the present invention, the siRNA composition reduces the expression of a BRC nucleic acid selected from the group consisting of BRC-associated genes A5657, B9769 and C7965. In yet another method, the treatment or prevention of BRC in a subject may be carried out by administering to a subject a ribozyme composition. In the context of the present invention, the nucleic acid-specific ribozyme composition reduces the expression of a BRC nucleic acid selected from the group consisting of the BRC-associated genes A5657, B9769 and C7965.

The present invention also includes vaccines and vaccination methods. For example, a method of treating or preventing BRC in a subject may involve administering to the subject a vaccine containing a polypeptide encoded by a nucleic acid selected from the group consisting of the BRC-associated genes A5657, B9769 and C7965 or an immunologically active fragment of such polypeptide. In the context of the present invention, an immunologically active fragment is a polypeptide that is shorter in length than the full-length naturally-occurring protein yet which induces an immune response analogous to that induced by the full-length protein. For example, an immunologically active fragment should be at least 8 residues in length and capable of stimulating an immune cell such as a T cell or a B cell. Immune cell stimulation can be measured by detecting cell proliferation, elaboration of cytokines (e.g., IL-2), induction of cytotoxic T lymphocyte, or production of an antibody.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference herein in their entirety. In case of conflict, the present specification, including definitions, will control. In addition, the materials, methods, and examples are illustrative only and not intended to be limiting.

One advantage of the methods described herein is that the disease is identified prior to detection of overt clinical symptoms of breast cancer. Other features and advantages of the invention will be apparent from the following detailed description, and from the claims.

### BRIEF DESCRIPTION OF THE FIGURES

FIG. 1 depicts the results of semi-quantitative RT-PCR. Expression levels of (a) A5657, (b) B9769 and (c) C7965 in tumor cells from breast cancer patients (3T, 31T, 149T, 175T, 431T, 453T, 491T, 554T, 571T, 709T, 772T and 781T), breast cancer cell lines (HBC4, HBC5, HBL100, HCC1937, MCF7, MDA-MB-231, SKBR3, T47D, YMB1, BT-20, BT-474, BT-549, HCC1143, HCC1500, HCC1599, MDA-MB-157, MDA-MB-435S, MDA-MB-453, OCUB-F and ZR-75-1), and normal human tissues are shown. Pre refers to normal breast ductal cells, MG to mammary gland, LUN to lung, LIV to liver, HEA to heart, KID to kidney and BM to bone marrow.

FIG. 2 depicts the results of Northern blot analysis of (a) A5657, (b) B9769 and (c) C7965 transcripts in various human tissues (upper panel), and breast cancer cell lines and normal human vital organs (bottom panel).

FIG. 3 depicts the subcellular localization of exogenous (a) A5657, (b) B9769 (upper panel; low density of cells, bottom panel, high density of cells) and (d) C7965 and control Mock in T47D breast cancer cells. Part (c) depicts the subcellular localization of exogenous B9769 as compared to other cytoskeleton proteins.

FIG. 4 depicts the growth-inhibitory effects of small-interfering RNAs (siRNAs) designed to reduce expression of A5657 in breast cancer cells. Part (a) depicts the results of semi-quantitative RT-PCR demonstrating suppression of endogenous expression of A5657 in breast cancer cell (T47D) at 28-day cultures in selective medium containing neomycin after introduction of siRNAs into T47D cells. GAPDH was used as an internal control. Part (b) depicts the results of an MTT assay demonstrating a decrease in the numbers of colonies by knockdown of A5657 in T47D cells. Part (c) depicts the results of a colony-formation assay demonstrating a decrease in the numbers of colonies by knockdown of A5657 in T47D cells.

FIG. 5 depicts the growth-inhibitory effects of small-interfering RNAs (siRNAs) designed to reduce expression of B9769 in breast cancer cells. Part (a) depicts the results of semi-quantitative RT-PCR showing suppression of endogenous expression of B9769 in breast cancer cell (T47D) at 28-day cultures in selective medium containing neomycin after introduction of siRNAs into T47D cells. GAPDH was used as an internal control. Part (b) depicts the results of an MTT assay demonstrating a decrease in the numbers of colonies by knockdown of B9769 in T47D cells. Part (c) depicts the results of a colony-formation assay demonstrating a decrease in the numbers of colonies by knockdown of B9769 in T47D cells.

FIG. 6 depicts the growth-inhibitory effects of small-interfering RNAs (siRNAs) designed to reduce expression of C7965 in breast cancer cells. Part (a) depicts the results of semi-quantitative RT-PCR showing suppression of endogenous expression of C7965 in breast cancer cell (T47D) at 28-day cultures in selective medium containing neomycin after introduction of siRNAs into T47D cells. GAPDH was used as an internal control. Part (b) depicts the results of an MTT assay demonstrating a decrease in the numbers of colonies by knockdown of C7965 in T47D cells. Part (c) depicts the results of a colony-formation assay demonstrating a decrease in the numbers of colonies by knockdown of C7965 in T47D cells.

FIG. 7 demonstrates that the over-expression of A5657, B9769 and C7965 increases the rate of cell proliferation. NIH3T3 cells were plated on 6-well plates and transiently transfected with A5657, B9769 and C7965 expression vectors as indicated, respectively. MTT assays were performed to monitor the value of O.D. for cell proliferation at 1, 2,4 and 6 days. Bars show mean±S.E. (n=0.2).

FIG. 8 depicts the ubiquitination of the A5657 protein. Part (A) depicts the results of an in vivo ubiquitination assay. Cell lysates were subjected directly to anti-Flag blotting (left) or immunoprecipitated with anti-HA antibody followed by anti-Flag immunoblotting (right). Part (B) demonstrates cell lysates subjected directly to anti-HA blotting (left) or immunoprecipitated with anti-FLAG antibody followed by anti-HA immunoblotting (right). Ubiquitins were conjugated to FLAG-A5657. An extra band on the Western blot indicated A5657 with ubiquitination.

### DETAILED DESCRIPTION

The words “a”, “an” and “the” as used herein mean “at least one” unless otherwise specifically indicated.

Generally breast cancer cells exist as a solid mass having a highly inflammatory reaction and containing various cellular components. Therefore, previous published microarray data are likely to reflect heterogenous profiles.

With these issues in view, the present inventers prepared purified populations of breast cancer cells and normal breast epithelial duct cells by a method of laser-microbeam microdissection (LMM. The gene-expression profiles of cancer cells from 81 BRCs, including 12 DCISs and 69 IDCs, were analyzed using a cDNA microarray representing 23,040 genes. By comparing expression patterns between cancer cells from patients diagnosed with BRC and normal ductal epithelial cells purely selected with Laser Microdissection, 102 genes (data not shown) were identified as commonly up-regulated in BRC cells, and 288 genes (data not shown) were identified as being commonly down-regulated in BRC cells. Candidate molecular markers having the potential to detect cancer-related proteins in serum of patients were selected, and some potential targets for development of signal-suppressing strategies in human BRC were discovered. In particular, the present invention involves the discovery of changes in expression patterns of three nucleic acids, namely A5657, B9769 and C7965, between epithelial cells and carcinomas of patients with BRC. The A5657 gene (SEQ ID NO: 1) constitutes a novel sequence and encodes an HSPC150 protein (SEQ ID NO: 2) similar to ubiquitin-conjugating enzyme (Genbank Accession No. NM_{—}014176). A5657 is up-regulated in IDC cells as compared to DCIS cells and normal breast epithelial cells. The B9769 gene (SEQ ID NO: 3) constitutes a novel sequence and encodes a hypothetical protein (SEQ ID NO: 4) (Genbank Accession No. NM_{—}138770). B9769 is up-regulated in IDC cells as compared to normal breast epithelial cells. The C7965 gene (SEQ ID NO: 5), and its encoded protein (SEQ ID NO: 6) constitute known sequences designated as LOC90557. C7965 is up-regulated in both DCIS cells and IDC cells as compared to normal breast epithelial cells. The differentially expressed genes identified herein find diagnostic utility as markers of BRC and as BRC gene targets, the expression of which may be altered to treat or alleviate a symptom of BRC. Alternatively, the A5657 gene, differentially expressed between DCIS and IDC, identified herein finds diagnostic utility as a marker for distinguishing IDC from DCIS as well as a BRC gene target, the expression of which may be altered to treat or alleviate a symptom of IDC.

These genes, whose expression level is modulated (i.e., increased) in BRC patients, are collectively referred to herein as “BRC-associated genes”, “BRC nucleic acids” or “BRC polynucleotides” and the corresponding encoded polypeptides are referred to as “BRC polypeptides” or “BRC proteins.” Unless indicated otherwise, “BRC” refers to any of the sequences disclosed herein (e.g., BRC-associated genes selected from the group consisting of A5657, B9769, and C7965).

By measuring expression of the various genes in a sample of cells, BRC can be diagnosed. Similarly, measuring the expression of these genes in response to various agents can identify agents for treating BRC.

The present invention involves determining (e.g., measuring) the expression of at least one, and up to all the BRC-associated genes selected from the group consisting of A5657, B9769, and C7965. Using sequence information provided by the GenBank™ database entries for known sequences, the BRC-associated genes can be detected and measured using techniques well known to one of ordinary skill in the art. For example, sequences within the sequence database entries corresponding to BRC-associated genes, can be used to construct probes for detecting RNA sequences corresponding to BRC-associated genes in, e.g., Northern blot hybridization analyses. Probes typically include at least 10, at least 20, at least 50, at least 100, or at least 200 nucleotides of a reference sequence. As another example, the sequences can be used to construct primers for specifically amplifying the BRC nucleic acid in, e.g., amplification-based detection methods, such as reverse-transcription based polymerase chain reaction.

Expression level of one or more of BRC-associated genes in a test cell population, e.g., a patient-derived tissues sample, is then compared to the expression level(s) of the same gene(s) in a reference population. The reference cell population includes one or more cells for which the compared parameter is known, ie., breast ductal carcinoma cells (e.g., BRC cells) or normal breast ductal epithelial cells (e.g., non-BRC cells).

Whether or not a pattern of gene expression in a test cell population as compared to a reference cell population indicates BRC or a predisposition thereto depends upon the composition of the reference cell population. For example, if the reference cell population is composed of non-BRC cells, a similarity in gene expression pattern between the test cell population and the reference cell population indicates the test cell population is non-BRC. Conversely, if the reference cell population is made up of BRC cells, a similarity in gene expression profile between the test cell population and the reference cell population indicates that the test cell population includes BRC cells.

A level of expression of a BRC marker gene in a test cell population is considered “altered” if it varies from the expression level of the corresponding BRC marker gene in a reference cell population by more than 1.0, more than 1.5, more than 2.0, more than 5.0, more than 10.0 or more fold.

Differential gene expression between a test cell population and a reference cell population can be normalized to a control nucleic acid, e.g. a housekeeping gene. For example, a control nucleic acid is one which is known not to differ depending on the cancerous or non-cancerous state of the cell. The expression level of a control nucleic acid can be used to normalize signal levels in the in the test and reference populations. Exemplary control genes include, but are not limited to, e.g., β-actin, glyceraldehyde 3-phosphate dehydrogenase and ribosomal protein P1.

The test cell population can be compared to multiple reference cell populations. Each of the multiple reference populations may differ in the known parameter. Thus, a test cell population may be compared to a first reference cell population known to contain, e.g., BRC cells, as well as a second reference population known to contain, e.g., non-BRC cells (normal cells). The test cell may be included in a tissue type or cell sample from a subject known to contain, or suspected of containing, BRC cells.

The test cell is obtained from a bodily tissue or a bodily fluid, e.g., biological fluid (such as blood or sputum, for example). For example, the test cell may be purified from breast tissue. Preferably, the test cell population comprises an epithelial cell. The epithelial cell is preferably from a tissue known to be or suspected to be a breast ductal carcinoma.

Cells in the reference cell population should be derived from a tissue type similar to that of the test cell. Optionally, the reference cell population is a cell line, e.g. a BRC cell line (i.e., a positive control) or a normal non-BRC cell line (i.e., a negative control). Alternatively, the control cell population may be derived from a database of molecular information derived from cells for which the assayed parameter or condition is known.

The subject is preferably a mammal. Exemplary mammals include, but are not limited to, e.g., a human, non-human primate, mouse, rat, dog, cat, horse, or cow.

Expression of the genes disclosed herein can be determined at the protein or nucleic acid level, using methods known in the art. For example, Northern hybridization analysis, using probes which specifically recognize one or more of these nucleic acid sequences can be used to determine gene expression. Alternatively, gene expression may be measured using reverse-transcription-based PCR assays, e.g., using primers specific for the differentially expressed gene sequences. Expression may also be determined at the protein level, i.e., by measuring the level of a polypeptides encoded by a gene described herein, or the biological activity thereof. Such methods are well known in the art and include, but are not limited to, e.g., immunoassays that utilize antibodies to proteins encoded by the genes. The biological activities of the proteins encoded by the genes are either generally well known or can be routinely identified.

Novel Nucleotides, Polypeptides, Vectors and Host Cells:

The present invention provides novel human genes, A5657 and B9769, whose expression is markedly elevated in a great majority of breast carcinomas as compared to corresponding non-cancerous breast epithelia. The isolated A5657 gene includes a polynucleotide sequence as described in SEQ ID NO: 1, a cDNA sequence containing 928 nucleotides. In particular, A5657 consists of 7 exons and is located on the chromosome 1q32.1 spanning approximately 10.3 kb in the genome. The cDNA transcript eventually encodes a polypeptide of 197 amino acids, an HSPC150 protein similar to ubiquitin-conjugating enzyme. The isolated B9769 gene includes a polynucleotide as described in SEQ ID NO: 3, a cDNA sequence containing 1472 nucleotides. In particular, B9769 consists of 8 exons and is located on the chromosome 2q21.2 spanning approximately 5.7 kb in the genome. The ORF starts at exon 1 and ends at exon 8. Eventually, the cDNA transcript encodes a polypeptide of 378 amino acids.

In addition to the novel human genes A5657 and B9769, including the polynucleotide sequences described in SEQ ID NOs: 1 and 3, the present invention encompasses degenerates and mutants thereof, to the extent that they encode an A5657 or B9769 protein, including the amino acid sequences set forth in SEQ ID NOs: 2 and 4 or functional equivalents thereof. Examples of polypeptides functionally equivalent to A5657 or B9769 include, for example, homologous proteins of other organisms corresponding to the human A5657 or B9769 proteins, as well as mutants of such human proteins.

The present invention further encompasses polypeptides that are functionally equivalent to the polypeptides identified by the present inventors and polynucleotides encoding such functionally equivalent polypeptides. In the context of the present invention, the term “functionally equivalent” means that the subject polypeptide retains a biologically significant activity that is characteristic of the A5657 or B9769 protein, the amino acid sequences of which are shown in SEQ ID NOs: 2 and 4, respectively. For example, both the A5657 and B9769 genes are characterized as being specifically overexpressed in BRC cells as compared to normal cells. The A5657 gene is further overexpressed in IDC cells as compared to DCIS cells. Moreover, their overexpression is demonstrated herein to promote cell proliferation. Accordingly, in the context of the present invention, a functional equivant of the A5657 or B9769 protein should have a cell proliferative activity analogous to that of the wild-type protein. Cell proliferation is a parameter that may be measured using assays and techniques conventional in the art (e.g., the MTT assay discussed in the Examples below). In addition, the A5657 protein is demonstrated herein to have E2 ubiquitin enzyme activity and to bind to ubiquitin. Accordingly, in the context of the instant invention, a functional equivant of the A5657 protein should have ubiquitin enzyme and/or binding activities analogous to that of the wild-type protein.

Accordingly, the present invention contemplates certain mutations or variants of the disclosed sequences. For example, the present invention encompasses polynucleotides encoding a protein of SEQ ID NO: 2 or 4, in which one or more amino acids are substituted, deleted, inserted and/or added, so long as the resulting protein retains a biologically significant activity of the wild-type protein. In a preferred embodiment, the functionally equivalent protein encoded by the polynucleotide of the present invention is similarly overexpressed in BRC cells as compared to normal cells. The present invention also includes polynucleotides that hybridize under stringent conditions with an A5657 or B9769 DNA, including the nucleotide sequence of SEQ ID NOs: 1 and 3, respectively, so long as the resulting polynucleotide encodes a protein that is functionally equivalent to the A5657 or B9769 protein. The determination of biologically significant activity can be conducted by methods well known to those skilled in the art, including methods described herein (see Examples section).

The invention provides an isolated polynucleotide that encodes a polypeptide described herein or a fragment thereof. Preferably, the isolated polypeptide is encoded by a nucleotide sequence that is at least about 60% identical to the nucleotide sequence shown in SEQ ID NO: 1 or 3. More preferably, the isolated nucleic acid molecule is at least about 65%, 70%, 75%, 80%, 85%, 90%, 91%, 92%, 93%, 94%, 95%, 96%, 97%, 98%, 99%, or more, identical to the nucleotide sequence shown in SEQ ID NO: 1 or 3.

To determine the percent identity of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions) multiplied by 100). The percent identity between two sequences can be determined using conventional techniques such as to those described herein, with or without allowing gaps. In calculating percent identity, typically exact matches are counted.

The determination of percent identity between two sequences can be accomplished using any conventional mathematical algorithm, such as the BLAST algorithm by Karlin and Altschul (S. Karlin and S. F. Altschul, Proc. Natl. Acad. Sci. USA. 1990, 87: 2264-2268; S. Karlin and S. F. Altschul, Proc. Natl. Acad. Sci. USA. 1993, 90: 5873-5877). The BLAST algorithm is incorporated into the BLASTN and BLASTX programs of Altschul et al. (S. F. Altschul et al., J. Mol. Biol. 1990, 215: 403). When a nucleotide sequence is analyzed according to BLASTN, suitable parameters include, for example, a score=100 and word length=12. On the other hand, suitable parameters for the analysis of amino acid sequences by BLASTX include, for example, a score=50 and word length-3. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. When utilizing BLAST, Gapped BLAST, and PSI-Blast programs, the default parameters of the respective programs (e.g., XBLAST and NBLAST) are preferably used. However, one skilled in the art can readily adjust the parameters to suit a particular purpose.

Another example of a mathematical algorithm that may be utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

Methods for preparing polypeptides functionally equivalent to a given protein are well known to those skilled in the art and include conventional methods of introducing mutations into a protein. For example, one skilled in the art can prepare polypeptides functionally equivalent to the A5657 or B9769 protein by introducing an appropriate mutation in the amino acid sequence of either of these proteins by site-directed mutagenesis (Hashimoto-Gotoh et al., Gene 152:271-5 (1995); Zoller and Smith, Methods Enzymol 100: 468-500 (1983); Kramer et al., Nucleic Acids Res. 12:9441-9456 (1984); Kramer and Fritz, Methods Enzymol 154: 350-67 (1987); Kunkel, Proc Natl Acad Sci USA 82: 488-92 (1985); Kunkel, Methods Enzymol 85: 2763-6 (1988)). Mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Amino acid mutations can occur in nature, too. Accordingly, the present invention includes proteins having the amino acid sequences of the A5657 or B9769 protein in which one or more amino acids are mutated, provided the resulting mutated polypeptide is functionally equivalent to the wild-type A5657 or B9769 protein.

The number of amino acids that may be mutated is not particularly restricted, so long as biologically significant activity is maintained. Generally, up to about 50 amino acids may be mutated, preferably up to about 30 amino acids, more preferably up to about 10 amino acids, and even more preferably up to about 3 to 5 amino acids. Likewise, the site of mutation is not particularly restricted, so long as the mutation does not result in the disruption of biologically significant activity.

Amino acid substitutions may be made at one or more predicted, preferably nonessential amino acid residues. A “nonessential” amino acid residue is a residue that can be altered from the wild-type sequence of a protein (e.g., the sequences shown in SEQ ID NOs: 2 and 4) without altering the biological activity, whereas an “essential” amino acid residue is required for biological activity. The amino acid residue to be mutated is preferably substituted for a different amino acid that allows the properties of the amino acid side-chain to be conserved (a process known as conservative amino acid substitution). Herein, the phrase “conservative amino acid substitution” refers to the replacement of an amino acid residue is replaced with an amino acid residue having a chemically similar side chain. Groups of amino acid residues having similar side chains have been defined in the art. Examples of amino acid groups include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Additional examples of amino acid groupings include side chains having the following chains properties, characteristics, and/or functional groups or characteristics in common: hydrophobic amino acids (A, I, L, M, F, P, W, Y, V), hydrophilic amino acids (R, D, N, C, E, Q, G, H, K, S, T), aliphatic side-chains (G, A, V, L, I, P); a hydroxyl containing side-chains (S, T, Y); sulfur atom containing side-chains (C, M); carboxylic acid and amide containing side-chains (D, N, E, Q); base containing side-chains (R, K, H); and aromatic containing side-chains (H, F, Y, W). Note, the parenthetic letters indicate the one-letter codes of amino acids. The number of amino acids that may be mutated is not particularly restricted, so long as the activity is maintained. Generally, up to about 50 amino acids may be mutated, preferably up to about 30 amino acids, more preferably up to about 10 amino acids, and even more preferably up to about 3 amino acids. Likewise, the site of mutation is not particularly restricted, so long as the mutation does not result in the disruption of the activity. Alternatively, the number of mutations typically corresponds to 30% or less, or 20% or less, or 10% or less, preferably 5% or less, or 3% or less of the total amino acids, more preferably 2% or less or 1% or less of the total amino acids.

An example of a polypeptide in which one or more amino acids residues are added to the amino acid sequence of an A5657 or B9769 protein is a fusion protein containing the A5657 or B9769 protein. Fusion proteins are fusions of an A5657 or B9769 protein with other peptides or proteins, and are included in the present invention. Fusion proteins can be made by techniques well known to a person skilled in the art, such as by linking the DNA encoding an A5657 or B9769 protein of the present invention with DNA encoding other peptides or proteins, so that the frames match, inserting the fusion DNA into an expression vector and expressing it in a host. There is no restriction as to the peptides or proteins fused to the protein of the present invention.

Known peptides that can be used as peptides that are fused to the protein of the present invention include, for example, FLAG (Hopp et al., Biotechnology 6: 1204-10 (1988)), 6×H is containing six H is (histidine) residues, 10×His, Influenza agglutinin (HA), human c-myc fragment, VSP-GP fragment, p18HIV fragment, T7-tag, HSV-tag, E-tag, SV40T antigen fragment, Ick tag, α-tubulin fragment, B-tag, Protein C fragment, and the like. Examples of proteins that may be fused to a protein of the invention include GST (glutathione-S-transferase), Influenza agglutinin (HA), immunoglobulin constant region, β-galactosidase, MBP (maltose-binding protein), and such.

Fusion proteins can be prepared by fusing commercially available DNA, encoding the fusion peptides or proteins discussed above, with the DNA encoding the polypeptide of the present invention and expressing the fused DNA prepared.

An alternative method known in the art for isolating functionally equivalent polypeptides involves conventional hybridization techniques (Sambrook et al., Molecular Cloning 2nd ed. 9.47-9.58, Cold Spring Harbor Lab. Press (1989)). One skilled in the art can readily isolate a DNA having high homology with a whole or part of the DNA sequence encoding the human A5657 or B9769 protein (i.e., SEQ ID NO: 1 or 3), and isolate polypeptides functionally equivalent to the human A5657 or B9769 protein from the isolated DNA. The polypeptides of the present invention include those that are encoded by DNA that hybridize with a whole or part of the DNA sequence encoding the A5657 or B9769 protein and are functionally equivalent to the A5657 or B9769 protein. These polypeptides include mammal homologues corresponding to the protein derived from human (for example, a polypeptide encoded by a monkey, rat, rabbit and bovine gene).

The condition of hybridization for isolating a DNA encoding a polypeptide functionally equivalent to the A5657 or B9769 protein can be routinely selected by a person skilled in the art. For example, hybridization may be performed by conducting prehybridization at 68° C. for 30 min or longer using “Rapid-hyb buffer” (Amersham LIFE SCIENCE), adding a labeled probe, and warming at 68° C. for 1 hour or longer. The following washing step can be conducted, for example, in a low stringent condition. A low stringent condition is, for example, 42° C., 2×SSC, 0.1% SDS, or preferably 50° C., 2×SSC, 0.1% SDS. More preferably, high stringent conditions are used. A high stringent condition is, for example, washing 3 times in 2×SSC, 0.01% SDS at room temperature for 20 min, then washing 3 times in 1×SSC, 0.1% SDS at 37° C. for 20 min, and washing twice in 1×SSC, 0.1% SDS at 50° C. for 20 min. However, several factors, such as temperature and salt concentration, can influence the stringency of hybridization and one skilled in the art can suitably select the factors to achieve the requisite stringency.

In place of hybridization, a gene amplification method, for example, the polymerase chain reaction (PCR) method, can be utilized to isolate a DNA encoding a polypeptide functionally equivalent to the A5657 or B9769 protein, using a primer synthesized based on the sequence information of the protein encoding DNA (SEQ ID NO: 1 or 3).

Polypeptides that are functionally equivalent to the A5657 or B9769 protein encoded by the DNA isolated through the above hybridization techniques or gene amplification techniques, normally have a high homology to the amino acid sequence of the human A5657 or B9769 protein. Herein, “high homology” typically refers to a homology of 40% or higher, preferably 60% or higher, more preferably 80% or higher, even more preferably 85%, 90% or 95% or higher. The homology of a polypeptide can be determined by following the algorithm in “Wilbur and Lipman, Proc Natl Acad Sci USA 80: 726-30 (1983)”.

A polypeptide of the present invention may have variations in amino acid sequence, molecular weight, isoelectric point, the presence or absence of sugar chains, or form, depending on the cell or host used to produce it or the purification method utilized. Nevertheless, so long as it has a function equivalent to that of an A5657 or B9769 protein of the present invention, it is within the scope of the present invention.

The polypeptides of the present invention can be prepared as recombinant proteins or natural proteins, by methods well known to those skilled in the art. A recombinant protein can be prepared by inserting a DNA, which encodes the polypeptide of the present invention (for example, the DNA comprising the nucleotide sequence of SEQ ID NO: 1 or 3), into an appropriate expression vector, introducing the vector into an appropriate host cell, obtaining the extract, and purifying the polypeptide by subjecting the extract to chromatography, for example, ion exchange chromatography, reverse phase chromatography, gel filtration, or affinity chromatography utilizing a column to which antibodies against the protein of the present invention is fixed, or by combining more than one of aforementioned columns. Also when the polypeptide of the present invention is expressed within host cells (for example, animal cells and E. coli) as a fusion protein with glutathione-5-transferase protein or as a recombinant protein supplemented with multiple histidines, the expressed recombinant protein can be purified using a glutathione column or nickel column. Alternatively, when the polypeptide of the present invention is expressed as a protein tagged with c-myc, multiple histidines, or FLAG, it can be detected and purified using antibodies to c-myc, H is, or FLAG, respectively.

After purifying the fusion protein, it is also possible to exclude regions other than the objective polypeptide by cutting with thrombin or factor-Xa as required. A natural protein can be isolated by methods known to a person skilled in the art, for example, by contacting the affinity column, in which antibodies binding to the A5657 or B9769 protein described below are bound, with the extract of tissues or cells expressing the polypeptide of the present invention. The antibodies can be polyclonal antibodies or monoclonal antibodies.

The present invention also encompasses partial peptides of polypeptides of the present invention. The partial peptide has an amino acid sequence specific to a polypeptide of the present invention and consists of at least 7 amino acids, preferably 8 amino acids or more, and more preferably 9 amino acids or more. The partial peptide can be used, for example, for preparing antibodies against the polypeptide of the present invention, screening for a compound that binds to the polypeptide of the present invention, and screening for accelerators or inhibitors of the polypeptide of the present invention.

A partial peptide of the invention can be produced by genetic engineering, by known methods of peptide synthesis, or by digesting the polypeptide of the invention with an appropriate peptidase. For peptide synthesis, for example, solid phase synthesis or liquid phase synthesis may be used.

Furthermore, the present invention provides polynucleotides encoding the polypeptide of the present invention. Examples of polynucleotides of the present invention include DNA comprising the nucleotide sequence of SEQ ID NOs: 1 or 3. The polynucleotides of the present invention can be used for the in vivo or in vitro production of the polypeptide of the present invention as described above, or can be applied to gene therapy for diseases attributed to genetic abnormality in the gene encoding the protein of the present invention. Any form of the polynucleotide of the present invention can be used so long as it encodes the polypeptide of the present invention, including mRNA, RNA, cDNA, genomic DNA, chemically synthesized polynucleotides. The polynucleotide of the present invention include a DNA comprising a given nucleotide sequences as well as its degenerate sequences, so long as the resulting DNA encodes a polypeptide of the present invention.

A polynucleotide of the present invention is preferably isolated. As used herein, an “isolated polynucleotide” is a polynucleotide removed from its original environment (e.g., the natural environment if naturally occurring) and thus, altered by the “hand of man” from its natural state, the structure of which is not identical to that of any naturally occurring nucleic acid or to that of any fragment of a naturally occurring genomic nucleic acid spanning more than three genes. The term therefore covers, for example, (a) a DNA which fragment has the sequence of part of a naturally occurring genomic DNA molecule free of the coding sequences that naturally flank the nucleic acid (i.e., sequences located at the 5′ and 3′ ends of the nucleic acid) in the genomic DNA but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in random, uncharacterized mixtures of different DNA molecules, transfected cells, or cell clones, e.g., as these occur in a DNA library such as a cDNA or genomic DNA library.

Polynucleotides of the present invention can be isolated by methods well known to those skilled in the art. Exemplary methods include the hybridization techniques discussed above (E. M. Southern, J. Mol. Biol. 1975, 98: 503-517) as well as the polymerase chain reaction (PCR) technique (R. K. Saiki et al., Science 1985, 230: 1350-1354; R. K. Saiki et al., Science 1988, 239: 487-491). More specifically, those skilled in the art can generally isolate polynucleotides highly homologous to the polynucleotide shown in SEQ ID NOs: 1 and 3 from other animals (such as human), using the polynucleotide shown in SEQ ID NOs: 1 and 3 or a part thereof as probes or using the oligonucleotide which specifically hybridizes with the polynucleotide shown in SEQ ID NOs: 1 and 3 as primers. Furthermore, polynucleotides that can be isolated by hybridization techniques or PCR techniques and that hybridize with polynucleotides shown in SEQ ID NOs: 1 and 3 are also included in the polynucleotides of the present invention.

Hybridization reactions to isolate polynucleotides as described above are preferably conducted under stringent conditions. Hybridization may be performed with buffers that permit the formation of a hybridization complex between nucleic acid sequences that contain some mismatches. At high stringency, hybridization complexes will remain stable only where the nucleic acid molecules are almost completely complementary. Many factors determine the stringency of hybridization, including G+C content of the cDNA, salt concentration, and temperature. For example, stringency may be increased by reducing the concentration of salt or by raising the hybridization temperature. Temperature conditions for hybridization and washing greatly influence stringency and can be adjusted using melting temperature (Tm). Tm varies with the ratio of constitutive nucleotides in the hybridizing base pairs, and with the composition of the hybridization solution (concentrations of salts, formamide and sodium dodecyl sulfate). In solutions used for some membrane based hybridizations, addition of an organic solvent, such as formamide, allows the reaction to occur at a lower temperature. Accordingly, on considering the relevant parameters, one skilled in the art can select appropriate conditions to achieve a suitable stringency based experience or experimentation.

Examples of stringent hybridization conditions includes conditions comprising: 6 M urea, 0.4% SDS and 0.5×SSC, and those having a stringency equivalent to the conditions. Polynucleotides with higher homology are expected to be isolated when hybridization is performed under conditions with higher stringency, for example, 6 M urea, 0.4% SDS and 0.1×SSC. Polynucleotides isolated under higher stringency conditions, such as described above, are expected to encode a polypeptide having a higher homology at the amino acid level to the amino acid sequence shown in SEQ ID NOs: 2 and 4. As noted above, “high homology” refers to an identity of at least 40% or more, preferably 60% or more, more preferably 80% or more, and even more preferably 85%, 90% or 95% or more, in the whole amino acid sequence.

The present invention also includes allelic variants of the polynucleotides shown in SEQ ID NOs: 1 and 3. An allele is one of two or more alternate forms of a gene occupying the same locus in a particular chromosome or linkage structure and differing from other alleles of the locus at one or more mutational sites. Accordingly, an allelic variant comprises an alteration in the normal sequence of a gene. Complete gene sequencing often identifies numerous allelic variants (sometimes hundreds) for a given gene. Allelic variants have a high percent identity to the original polynucleotide and may differ, for example, by about three bases per hundred bases. Allelic variants generally encode substantially identical proteins and typically do not affect the resulting phenotype. Allelic variants of a particular polynucleotide can be routinely obtained, for example, via the hybridization techniques discussed above.

Examples of allelic variants include genetic polymorphisms. Polymorphisms comprise differences in DNA sequences that exist among individuals. Genetic variations occurring in more than 1% of a population are considered useful polymorphisms for genetic linkage analysis. Certain polymorphisms (e.g., restriction fragment length polymorphisms (RFLP)) result in variations among individuals in DNA fragment sizes cut by specific enzymes, such as restriction enzymes. Polymorphic sequences that result in RFLPs are useful as markers on both physical maps and genetic linkage maps. Another example of a common genetic polymorphism is a “single nucleotide polymorphism” (SNP). SNPs comprise changes in a single base as a result of a substitution, insertion or deletion. The change may be conservative (purine for purine) or non-conservative (purine to pyrimidine) and may or may not result in a change in an encoded amino acid. Such changes may predispose an individual to a specific disease or condition.

The polynucleotide of the present invention can be prepared by methods known to a person skilled in the art. For example, the polynucleotide of the present invention can be prepared by: preparing a cDNA library from cells which express the polypeptide of the present invention, and conducting hybridization using a partial sequence of a DNA of the present invention (for example, SEQ ID NOs: 1 or 3) as a probe. A cDNA library can be prepared, for example, by the method described in Sambrook et al., Molecular Cloning, Cold Spring Harbor Laboratory Press (1989); alternatively, commercially available cDNA libraries may be used. A cDNA library can be also prepared by: extracting RNAs from cells expressing the polypeptide of the present invention, synthesizing oligo DNAs based on the sequence of a DNA of the present invention (for example, SEQ ID NOs: 1 or 3), conducting PCR using the oligo DNAs as primers, and amplifying cDNAs encoding the protein of the present invention.

In addition, by sequencing the nucleotides of the obtained cDNA, the translation region encoded by the cDNA can be routinely determined, and the amino acid sequence of the polypeptide of the present invention can be easily obtained. Moreover, by screening the genomic DNA library using the obtained cDNA or parts thereof as a probe, the genomic DNA can be isolated.

The obtained mRNA can be used to synthesize cDNA using reverse transcriptase. cDNA may be synthesized using a commercially available kit, such as the AMV Reverse Transcriptase First-strand cDNA Synthesis Kit (Seikagaku Kogyo). Alternatively, cDNA may be synthesized and amplified following the 5′-RACE method (Frohman et al., Proc Natl Acad Sci USA 85: 8998-9002 (1988); Belyavsky et al., Nucleic Acids Res 17: 2919-32 (1989)), which uses a primer and such, described herein, the 5′-Ampli FINDER RACE Kit (Clontech), and polymerase chain reaction (PCR).

A desired DNA fragment can be prepared from the PCR products and ligated with a vector DNA. The recombinant vectors are used to transform E. coli and such, and a desired recombinant vector is prepared from a selected colony. The nucleotide sequence of the desired DNA can be verified by conventional methods, such as dideoxynucleotide chain termination.

The nucleotide sequence of a polynucleotide of the invention may be designed to be expressed more efficiently by taking into account the frequency of codon usage in the host to be used for expression (Grantham et al., Nucleic Acids Res 9: 43-74 (1981)). The sequence of the polynucleotide of the present invention may be altered by a commercially available kit or a conventional method. For instance, the sequence may be altered by digestion with restriction enzymes, insertion of a synthetic oligonucleotide or an appropriate polynucleotide fragment, addition of a linker, or insertion of the initiation codon (ATG) and/or the stop codon (TAA, TGA, or TAG).

The present invention also provides a vector into which a polynucleotide of the present invention is inserted. A vector of the present invention is useful to keep a polynucleotide, especially a DNA, of the present invention in host cell, to express the polypeptide of the present invention, or to administer the polynucleotide of the present invention for gene therapy.

When E. coli is a host cell and the vector is amplified and produced in a large amount in E. coli (e.g., JM109, DH5α, HB101, or XL1Blue), the vector should have “ori” to be amplified in E. coli and a marker gene for selecting transformed E. coli (e.g., a drug-resistance gene selected by a drug such as ampicillin, tetracycline, kanamycin, chloramphenicol or the like). For example, M13-series vectors, pUC-series vectors, pBR322, pBluescript, pCR-Script, etc. can be used. In addition, pGEM-T, pDIRECT, and pT7 can also be used for subcloning and extracting cDNA as well as the vectors described above. When a vector is used to produce the protein of the present invention, an expression vector is especially useful. For example, an expression vector to be expressed in E. coli should have the above characteristics to be amplified in E. coli. When E. coli, such as JM109, DH5α, HB101, or XL1 Blue, are used as a host cell, the vector should have a promoter, for example, lacZ promoter (Ward et al., Nature 341: 544-6 (1989); FASEB J 6: 2422-7 (1992)), araB promoter (Better et al., Science 240: 1041-3 (1988)), or T7 promoter or the like, that can efficiently express the desired gene in E. coli. In that respect, pGEX-5×-1 (Pharmacia), “QIAexpress system” (Qiagen), pEGFP and pET (in this case, the host is preferably BL21 which expresses T7 RNA polymerase), for example, can be used instead of the above vectors. Additionally, the vector may also contain a signal sequence for polypeptide secretion. An exemplary signal sequence that directs the polypeptide to be secreted to the periplasm of the E. coli is the pelB signal sequence (Lei et al., J Bacteriol 169: 4379 (1987)). Means for introducing of the vectors into the target host cells include, for example, the calcium chloride method, and the electroporation method.

In addition to E. coli, for example, expression vectors derived from mammals (for example, pcDNA3 (Invitrogen) and pEGF-BOS (Nucleic Acids Res 18(17): 5322 (1990)), pEF, pCDM8), expression vectors derived from insect cells (for example, “Bac-to-BAC baculovirus expression system” (GIBCO BRL), pBacPAK8), expression vectors derived from plants (e.g., pMH1, pMH2), expression vectors derived from animal viruses (e.g., pHSV, pMV, pAdexLcw), expression vectors derived from retroviruses (e.g., pZIpneo), expression vector derived from yeast (e.g., “Pichia Expression Kit” (Invitrogen), pNV11, SP-Q01), and expression vectors derived from Bacillus subtilis (e.g., pPL608, pKTH50) can be used for producing the polypeptide of the present invention.

In order to express the vector in animal cells, such as CHO, COS, or NIH3T3 cells, the vector should have a promoter necessary for expression in such cells, for example, the SV40 promoter (Mulligan et al., Nature 277: 108 (1979)), the MMLV-LTR promoter, the EF1α promoter (Mizushima et al., Nucleic Acids Res 18: 5322 (1990)), the CMV promoter, and the like, and preferably a marker gene for selecting transformants (for example, a drug resistance gene selected by a drug (e.g., neomycin, G418)). Examples of known vectors with these characteristics include, for example, pMAM, pDR2, pBK-RSV, pBK-CMV, pOPRSV, and pOP13.

In addition, methods may be used to express a gene stably and, at the same time, to amplify the copy number of the gene in cells. For example, a vector comprising the complementary DHFR gene (e.g., pCHO I) may be introduced into CHO cells in which the nucleic acid synthesizing pathway is deleted, and then amplified by methotrexate (MTX). Furthermore, in case of transient expression of a gene, the method wherein a vector comprising a replication origin of SV40 (pcD, etc.) is transformed into COS cells comprising the SV40 T antigen expressing gene on the chromosome can be used.

A polypeptide of the present invention obtained as above may be isolated from inside or outside (such as medium) of host cells, and purified as a substantially pure homogeneous polypeptide. The term “substantially pure” as used herein in reference to a given polypeptide means that the polypeptide is substantially free from contaminants, such as other biological macromolecules, culture media (if recombinantly produced), or chemical precursors (if chemically synthesized). The substantially pure polypeptide is at least about 75%, preferably at least about 80%, more preferably at least about 85, 90, 95, or 99% (e.g., at least 80, 85, 95, or 99%) pure by dry weight. Purity can be measured by any appropriate standard conventional method, for example by column chromatography, polyacrylamide gel electrophoresis, or HPLC analysis.

For instance, column chromatography, filter, ultrafiltration, salt precipitation, solvent precipitation, solvent extraction, distillation, immunoprecipitation, SDS-polyacrylamide gel electrophoresis, isoelectric point electrophoresis, dialysis, and recrystallization may be appropriately selected and combined to isolate and purify the polypeptide.

Examples of chromatography include, for example, affinity chromatography, ion-exchange chromatography, hydrophobic chromatography, gel filtration, reverse phase chromatography, adsorption chromatography, and such (Strategies for Protein Purification and Characterization: A Laboratory Course Manual. Ed. Daniel R. Marshak et al., Cold Spring Harbor Laboratory Press (1996)). These chromatographies may be performed by liquid chromatography, such as HPLC and FPLC. Thus, the present invention provides for highly purified polypeptides prepared by the above methods.

A polypeptide of the present invention may be optionally modified or partially deleted by treating it with an appropriate protein modification enzyme before or after purification. Useful protein modification enzymes include, but are not limited to, trypsin, chymotrypsin, lysylendopeptidase, protein kinase, glucosidase, and so on.

Antibodies:

The present invention further provides antibodies that binds to a polypeptide of the invention. An antibody of the present invention can be used in any form, such as monoclonal or polyclonal antibodies, and includes antiserum obtained by immunizing an animal such as a rabbit with the polypeptide of the invention, all classes of polyclonal and monoclonal antibodies, human antibodies, and humanized antibodies produced by genetic recombination.

A polypeptide of the invention used as an antigen to obtain an antibody may be derived from any animal species, but preferably is derived from a mammal such as a human, mouse, or rat, more preferably from a human. A human-derived polypeptide may be obtained from the nucleotide or amino acid sequences disclosed herein.

According to the present invention, the polypeptide to be used as an immunization antigen may be a complete protein or a partial peptide of the protein. A partial peptide may comprise, for example, the amino (N)-terminal or carboxy (C)-terminal fragment of a polypeptide of the present invention.

Any mammalian animal may be immunized with the antigen, but preferably the compatibility with parental cells used for cell fusion is taken into account. In general, animals of the orders Rodentia, Lagomorpha, and Primate are preferably used. Animals of the order Rodentia include, but are not limited to, for example, mouse, rat, and hamster. Animals of the order Lagomorpha include, but are not limited to, for example, rabbit. Animals of the order Primate include, but are not limited to, for example, a monkey of the infra-order Catarrhini (old world monkey), such as Macaca fascicularis, rhesus monkey, sacred baboon, and chimpanzees.

Methods for immunizing animals with antigens are known in the art. Intraperitoneal injection or subcutaneous injection of antigens is a standard method for immunization of mammals. More specifically, antigens may be diluted and suspended in an appropriate amount of phosphate buffered saline (PBS), physiological saline, etc. If desired, the antigen suspension may be mixed with an appropriate amount of a standard adjuvant, such as Freund's complete adjuvant, made into emulsion, and then administered to mammalian animals. Preferably, it is followed by several administrations of antigen mixed with an appropriately amount of Freund's incomplete adjuvant every 4 to 21 days. An appropriate carrier may also be used for immunization. After immunization as above, serum is examined by a standard method for an increase in the amount of desired antibodies.

Polyclonal antibodies against the polypeptides of the present invention may be prepared by collecting blood from the immunized mammal examined for the increase of desired antibodies in the serum, and by separating serum from the blood by any conventional method. Polyclonal antibodies include serum containing the polyclonal antibodies, as well as the fraction containing the polyclonal antibodies may be isolated from the serum. Immunoglobulin G or M can be prepared from a fraction which recognizes only the polypeptide of the present invention using, for example, an affinity column coupled with the polypeptide of the present invention, and further purifying this fraction using protein A or protein G column.

To prepare monoclonal antibodies, immune cells are collected from the mammal immunized with the antigen and checked for the increased level of desired antibodies in the serum as described above, and are subjected to cell fusion. The immune cells used for cell fusion are preferably obtained from spleen. Other preferred parental cells to be fused with the above immunocyte include, for example, myeloma cells of mammalians, and more preferably myeloma cells having an acquired property for the selection of fused cells by drugs.

The above immunocyte and myeloma cells can be fused according to known methods, for example, the method of Milstein et al. (Galfre and Milstein, Methods Enzymol 73: 3-46 (1981)).

Resulting hybridomas obtained by the cell fusion may be selected by cultivating them in a standard selection medium, such as HAT medium (hypoxanthine, aminopterin, and thymidine containing medium). The cell culture is typically continued in the HAT medium for several days to several weeks, the time being sufficient to allow all the other cells, with the exception of the desired hybridoma (non-fused cells), to die. Then, the standard limiting dilution is performed to screen and clone a hybridoma cell producing the desired antibody.

In addition to the above method, in which a non-human animal is immunized with an antigen for preparing hybridoma, human lymphocytes such as those infected by EB virus may be immunized with a polypeptide, polypeptide expressing cells, or their lysates in vitro. Then, the immunized lymphocytes are fused with human-derived myeloma cells that are capable of indefinitely dividing, such as U266, to yield a hybridoma producing a desired human antibody that is able to bind to the polypeptide can be obtained (Unexamined Published Japanese Patent Application No. (JP-A) Sho 63-17688).

The obtained hybridomas are subsequently transplanted into the abdominal cavity of a mouse and the ascites are extracted. The obtained monoclonal antibodies can be purified by, for example, ammonium sulfate precipitation, a protein A or protein G column, DEAE ion exchange chromatography, or an affinity column to which the polypeptide of the present invention is coupled. The antibody of the present invention can be used not only for purification and detection of the polypeptide of the present invention, but also as a candidate for agonists and antagonists of the polypeptide of the present invention. In addition, this antibody can be applied to the antibody treatment for diseases related to the polypeptide of the present invention. When the obtained antibody is to be administered to the human body (antibody treatment), a human antibody or a humanized antibody is preferable for reducing immunogenicity.

For example, transgenic animals having a repertory of human antibody genes may be immunized with an antigen selected from a polypeptide, polypeptide expressing cells, or their lysates. Antibody producing cells are then collected from the animals and fused with myeloma cells to obtain hybridoma, from which human antibodies against the polypeptide can be prepared (see WO92-03918, WO93-2227, WO94-02602, WO94-25585, WO96-33735, and WO96-34096).

Alternatively, an immune cell, such as an immunized lymphocyte, producing antibodies may be immortalized by an oncogene and used for preparing monoclonal antibodies.

Monoclonal antibodies thus obtained can be also recombinantly prepared using genetic engineering techniques (see, for example, Borrebaeck and Larrick, Therapeutic Monoclonal Antibodies, published in the United Kingdom by MacMillan Publishers LTD (1990)). For example, a DNA encoding an antibody may be cloned from an immune cell, such as a hybridoma or an immunized lymphocyte producing the antibody, inserted into an appropriate vector, and introduced into host cells to prepare a recombinant antibody. The present invention also provides recombinant antibodies prepared as described above.

Furthermore, an antibody of the present invention may be a fragment of an antibody or modified antibody, so long as it binds to one or more of the polypeptides of the invention. For instance, the antibody fragment may be Fab, F(ab′)₂, Fv, or single chain Fv (scFv), in which Fv fragments from H and L chains are ligated by an appropriate linker (Huston et al., Proc Natl Acad Sci USA 85: 5879-83 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co et al., J Immunol 152: 2968-76 (1994); Better and Horwitz, Methods Enzymol 178: 476-96 (1989); Pluckthun and Skerra, Methods Enzymol 178: 497-515 (1989); Lamoyi, Methods Enzymol 121: 652-63 (1986); Rousseaux et al., Methods Enzymol 121: 663-9 (1986); Bird and Walker, Trends Biotechnol 9:132-7 (1991)).

An antibody may be modified by conjugation with a variety of molecules, such as polyethylene glycol (PEG). The present invention provides for such modified antibodies. The modified antibody can be obtained by chemically modifying an antibody. These modification methods are conventional in the field.

Alternatively, an antibody of the present invention may be obtained as a chimeric antibody, between a variable region derived from nonhuman antibody and the constant region derived from human antibody, or as a humanized antibody, comprising the complementarity determining region (CDR) derived from nonhuman antibody, the frame work region (FR) and the constant region derived from human antibody. Such antibodies can be prepared by using known technology.

Antibodies obtained as above may be purified to homogeneity. For example, the separation and purification of the antibody can be performed according to separation and purification methods used for general proteins. For example, the antibody may be separated and isolated by the appropriately selected and combined use of column chromatographies, such as affinity chromatography, filter, ultrafiltration, salting-out, dialysis, SDS polyacrylamide gel electrophoresis, isoelectric focusing, and others (Antibodies: A Laboratory Manual. Ed Harlow and David Lane, Cold Spring Harbor Laboratory (1988)), but are not limited thereto. A protein A column and protein G column can be used as the affinity column. Exemplary protein A columns to be used include, for example, Hyper D, POROS, and Sepharose F.F. (Pharmacia). Exemplary chromatography, with the exception of affinity includes, for example, ion-exchange chromatography, hydrophobic chromatography, gel filtration, reverse-phase chromatography, adsorption chromatography, and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC, and FPLC.

For example, measurement of absorbance, enzyme-linked immunosorbent assay (ELISA), enzyme immunoassay (EIA), radioimmunoassay (RIA), and/or immunofluorescence may be used to measure the antigen binding activity of the antibody of the invention. In ELISA, the antibody of the present invention is immobilized on a plate, a polypeptide of the invention is applied to the plate, and then a sample containing a desired antibody, such as culture supernatant of antibody producing cells or purified antibodies, is applied. Then, a secondary antibody that recognizes the primary antibody and is labeled with an enzyme, such as alkaline phosphatase, is applied, and the plate is incubated. Next, after washing, an enzyme substrate, such asp-nitrophenyl phosphate, is added to the plate, and the absorbance is measured to evaluate the antigen binding activity of the sample. A fragment of the polypeptide, such as a C-terminal or N-terminal fragment, may be used as the antigen to evaluate the binding activity of the antibody. BIAcore (Pharmacia) may be used to evaluate the activity of the antibody according to the present invention.

Diagnosing Breast Cancer:

In the context of the present invention, BRC is diagnosed by measuring the expression level of one or more BRC nucleic acids from a test population of cells, (i.e., a patient-derived biological sample). Preferably, the test cell population contains an epithelial cell, e.g., a cell obtained from breast tissue. Gene expression can also be measured from blood or other bodily fluids such as urine. Other biological samples can be used for measuring protein levels.

For example, the protein level in blood or serum derived from a subject to be diagnosed can be measured by immunoassay or other conventional biological assay. Expression of one or more BRC-associated genes, e.g., A5657, B9769 and C7965, is determined in the test cell or biological sample and compared to the normal control expression level associated with the one or more BRC-associated gene(s) assayed. A normal control level is an expression profile of a BRC-associated gene typically found in a population known not to be suffering from BRC. An alteration (e.g., an increase) in the level of expression in the patient-derived tissue sample of one or more BRC-associated gene indicates that the subject is suffering from or is at risk of developing BRC. For example, an increase in the expression of one or more of the BRC-associated genes A5657, B9769 and C7965 in the test population as compared to the normal control level indicates that the subject is suffering from or is at risk of developing BRC.

Increase in the level of expression of one or more of the BRC-associated genes in the test population as compared to the normal control level indicates that the subject suffers from or is at risk of developing BRC. For example, increase of one, two or three of the panel of BRC-associated genes (e.g., A5657, B9769 and C7965) indicates that the subject suffers from or is at risk of developing BRC.

Identifying Agents that Inhibit BRC-Associated Gene Expression:

An agent that inhibits the expression of a BRC-associated gene or the activity of its gene product can be identified by contacting a test cell population expressing a BRC-associated up-regulated gene with a test agent and then determining the expression level or activity of the BRC-associated gene. A decrease in the level of expression of the BRC-associated gene or in the level of activity of its gene product in the presence of the agent as compared to the normal control level (or compared to the expression or activity level in the absence of the test agent) indicates that the agent is an inhibitor of a BRC-associated gene and useful in inhibiting BRC.

The test cell population may be any cell expressing the BRC-associated genes. For example, the test cell population may contain an epithelial cell, such as a cell derived from breast tissue. Furthermore, the test cell may be an immortalized cell line derived from an carcinoma cell. Alternatively, the test cell may be a cell which has been transfected with a BRC-associated gene or which has been transfected with a regulatory sequence (e.g. promoter sequence) from a BRC-associated gene operably linked to a reporter gene.

Assessing Efficacy of Treatment of BRC in a Subject:

The differentially expressed BRC-associated genes identified herein also allow for the course of treatment of BRC to be monitored. In this method, a test cell population is provided from a subject undergoing treatment for BRC. If desired, test cell populations are obtained from the subject at various time points, before, during, and/or after treatment. Expression of one or more of the BRC-associated genes in the cell population is then determined and compared to a reference cell population which includes cells whose BRC state is known. In the context of the present invention, the reference cells should have not been exposed to the treatment of interest.

If the reference cell population contains no BRC cells, a similarity in the expression of a BRC-associated gene in the test cell population and the reference cell population indicates that the treatment of interest is efficacious. However, a difference in the expression of a BRC-associated gene in the test population and a normal control reference cell population indicates a less favorable clinical outcome or prognosis. Similarly, if the reference cell population contains BRC cells, a difference between the expression of a BRC-associated gene in the test cell population and the reference cell population indicates that the treatment of interest is efficacious, while a similarity in the expression of a BRC-associated gene in the test population and a control reference cell population indicates a less favorable clinical outcome or prognosis.

Additionally, the expression level of one or more BRC-associated genes determined in a subject-derived biological sample obtained after treatment (i.e., post-treatment levels) can be compared to the expression level of the one or more BRC-associated genes determined in a subject-derived biological sample obtained prior to treatment onset (i.e., pre-treatment levels). A decrease in the expression level in a post-treatment sample indicates that the treatment of interest is efficacious while an increase or maintenance in the expression level in the post-treatment sample indicates a less favorable clinical outcome or prognosis.

As used herein, the term “efficacious” indicates that the treatment leads to a reduction in the expression of a pathologically up-regulated gene, an increase in the expression of a pathologically down-regulated gene or a decrease in size, prevalence, or metastatic potential of breast ductal carcinoma in a subject. When a treatment of interest is applied prophylactically, the term “efficacious” means that the treatment retards or prevents a breast tumor from forming or retards, prevents, or alleviates a symptom of clinical BRC. Assessment of breast tumors can be made using standard clinical protocols.

In addition, efficaciousness can be determined in association with any known method for diagnosing or treating BRC. BRC can be diagnosed, for example, by identifying symptomatic anomalies, e.g., weight loss, abdominal pain, back pain, anorexia, nausea, vomiting and generalized malaise, weakness, and jaundice.

Selecting a Therapeutic Agent for Treating BRC that is Appropriate for a Particular Individual:

Differences in the genetic makeup of individuals can result in differences in their relative abilities to metabolize various drugs. An agent that is metabolized in a subject to act as an anti-BRC agent can manifest itself by inducing a change in a gene expression pattern in the subject's cells from that characteristic of a cancerous state to a gene expression pattern characteristic of a non-cancerous state. Accordingly, the differentially expressed BRC-associated genes disclosed herein allow for a putative therapeutic or prophylactic inhibitor of BRC to be tested in a test cell population from a selected subject in order to determine if the agent is a suitable inhibitor of BRC in the subject.

To identify an inhibitor of BRC that is appropriate for a specific subject, a test cell population from the subject is exposed to a therapeutic agent, and the expression of one or more of the BRC-associated genes A5657, B9769 and C7965 is determined.

In the context of the method of the present invention, the test cell population contains a BRC cell expressing a BRC-associated gene. Preferably, the test cell is an epithelial cell. For example, a test cell population may be incubated in the presence of a candidate agent and the pattern of gene expression of the test sample may be measured and compared to one or more reference profiles, e.g., a BRC reference expression profile or a non-BRC reference expression profile.

A decrease in expression of one or more of the BRC-associated genes A5657, B9769 and C7965 relative to a reference cell population containing BRC indicates that the agent has therapeutic potential.

In the context of the present invention, the test agent can be any compound or composition. Exemplary test agents include, but are not limited to, immunomodulatory agents.

Screening Assays for identifying Therapeutic Agents:

The differentially expressed BRC-associated genes disclosed herein can also be used to identify candidate therapeutic agents for treating BRC. The method of the present invention involves screening a candidate therapeutic agent to determine if it can convert an expression profile of one or more of the BRC-associated genes A5657, B9769 and C7965 characteristic of a BRC state to a gene expression pattern characteristic of a non-BRC state.

In the instant method, a cell is exposed to a test agent or a plurality of test agents (sequentially or in combination) and the expression of one or more of the BRC-associated genes A5657, B9769 and C7965 in the cell is measured. The expression profile of the BRC-associated gene(s) assayed in the test population is compared to expression level of the same BRC-associated gene(s) in a reference cell population that is not exposed to the test agent.

An agent capable of suppressing the expression of over-expressed or up-regulated genes has potential clinical benefit. Such agents may be further tested for the ability to prevent breast ductal carcinomal growth in animals or test subjects.

In a further embodiment, the present invention provides methods for screening candidate agents which are potential targets in the treatment of BRC. As discussed in detail above, by controlling the expression levels of marker genes or the activities of their gene products, one can control the onset and progression of BRC. Thus, candidate agents, which are potential targets in the treatment of BRC, can be identified through screening methods that use such expression levels and activities of as indices of the cancerous or non-cancerous state. In the context of the present invention, such screening may comprise, for example, the following steps:
a) contacting a test compound with a polypeptide encoded by A5657, B9769 and C7965;
b) detecting the binding activity between the polypeptide and the test compound; and
c) selecting the test compound that binds to the polypeptide.

Alternatively, the screening method of the present invention may comprise the following steps:
a) contacting a candidate compound with a cell expressing one or more marker genes, wherein the one or more marker genes is selected from the group consisting A5657, B9769 and C7965; and
b) selecting the candidate compound that reduces the expression level of one or more marker genes as compared to a control.
   Cells expressing a marker gene include, for example, cell lines established from BRC; such cells can be used for the above screening of the present invention.

Alternatively, the screening method of the present invention may comprise the following steps:
a) contacting a test compound with a polypeptide encoded by a polynucleotide selected from the group consisting of the BRC-associated genes A5657, B9769 and C7965;
b) detecting the biological activity of the polypeptide of step (a); and
c) selecting a compound that suppresses the biological activity of the polypeptide as compared to the biological activity detected in the absence of the test compound.

A protein for use in the screening method of the present invention can be obtained as a recombinant protein using the nucleotide sequence of the marker gene. Based on the information regarding the marker gene and its encoded protein, one skilled in the art can select any biological activity of the protein as an index for screening and any suitable measurement method to assay for the selected biological activity.

In another embodiment of the method for screening a compound for treating a cell proliferative disease of the present invention, the method utilizes biological activity of the polypeptide of the present invention as an index. Since the A5657, B9769 and C7965 proteins of the present invention have the activity of promoting cell proliferation, a compound which inhibits this activity of one of these proteins can be screened using this activity as an index. This screening method includes the steps of: (a) contacting a test compound with a polypeptide selected from group consisting of A5657, B9769 and C7965; (b) detecting the biological activity of the polypeptide of step (a); and (c) selecting a compound that suppresses the biological activity of the polypeptide in comparison with the biological activity detected in the absence of the test compound. In the present method, the polypeptide is preferably expressed in a living cell, and the biological activity can be detected by cell proliferation as the index. Furthermore, the preferable living cell is a host cell transfeted by polynucleotide selected from group consisting of A5657, B9769 and C7965. For example, NIH3T3 can be used as the host cell.

Any polypeptides can be used for screening so long as they comprise the biological activity of the A5657, B9769 and C7965 protein. Such biological activity include cell-proliferating activity of the human A5657, B9769 and C7965 protein. For example, a human A5657, B9769 and C7965 protein can be used and polypeptides functionally equivalent to these proteins can also be used. Such polypeptides may be expressed endogenously or exogenously by cells.

Alternatively, the screening method of the present invention may comprise the following steps:
a) contacting a candidate compound with a cell into which a vector, comprising the transcriptional regulatory region of one or more marker genes and a reporter gene that is expressed under the control of the transcriptional regulatory region, has been introduced, wherein the one or more marker genes are selected from the group consisting of the BRC-associated genes A5657, B9769 and C7965;
b) measuring the expression level or activity of said reporter gene; and
c) selecting the candidate compound that reduces the expression level or activity of said reporter gene as compared to a control

Suitable reporter genes and host cells are well known in the art. A reporter construct suitable for the screening method of the present invention can be prepared by using the transcriptional regulatory region of a marker gene. When the transcriptional regulatory region of the marker gene is known to those skilled in the art, a reporter construct can be prepared by using the previous sequence information. When the transcriptional regulatory region of the marker gene remains unidentified, a nucleotide segment containing the transcriptional regulatory region can be isolated from a genome library based on the nucleotide sequence information of the marker gene.

Any test compounds, for example, cell extracts, cell culture supernatant, products of fermenting microorganism, extracts of marine organism, plant extracts, purified or crude proteins, peptides, non-peptide compounds, synthetic micromolecular compounds, natural compounds, can be used.

A compound isolated by the screening serves as a candidate for the development of drugs that inhibit the activity of the protein encoded by marker gene and can be applied to the treatment or prevention of breast cancer.

Moreover, compounds in which a part of the structure of the compound inhibiting the activity of proteins encoded by marker genes is converted by addition, deletion and/or replacement are also included as the compounds obtainable by the screening method of the present invention.

When administrating a compound isolated by the method of the present invention as a pharmaceutical for humans and other mammals, such as mice, rats, guinea-pigs, rabbits, cats, dogs, sheep, pigs, cattle, monkeys, baboons, and chimpanzees, the isolated compound can be directly administered or can be formulated into a dosage form using known pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredient contained in such a preparation makes a suitable dosage within the indicated range acquirable.

Examples of additives that can be admixed into tablets and capsules include, but are not limited to, binders, such as gelatin, corn starch, tragacanth gum and arabic gum; excipients, such as crystalline cellulose; swelling agents, such as corn starch, gelatin and alginic acid; lubricants, such as magnesium stearate; sweeteners, such as sucrose, lactose or saccharin; and flavoring agents, such as peppermint, Gaultheria adenothrix oil and cherry. When the unit-dose form is a capsule, a liquid carrier, such as an oil, can be further included in the above ingredients. Sterile composites for injection can be formulated following normal drug implementations using vehicles, such as distilled water, suitable for injection.

Physiological saline, glucose, and other isotonic liquids, including adjuvants, such as D-sorbitol, D-mannnose, D-mannitol, and sodium chloride, can be used as aqueous solutions for injection. These can be used in conjunction with suitable solubilizers, such as alcohol, for example, ethanol; polyalcohols, such as propylene glycol and polyethylene glycol; and non-ionic surfactants, such as Polysorbate 80 (TM) and HCO-50.

Sesame oil or soy-bean oil can be used as an oleaginous liquid, may be used in conjunction with benzyl benzoate or benzyl alcohol as a solubilizer, and may be formulated with a buffer, such as phosphate buffer and sodium acetate buffer; a pain-killer, such as procaine hydrochloride; a stabilizer, such as benzyl alcohol and phenol; and/or an anti-oxidant. A prepared injection may be filled into a suitable ampoule.

Methods well known to those skilled in the art may be used to administer the pharmaceutical composition of the present invention to patients, for example as an intraarterial, intravenous, or percutaneous injection or as an intranasal, transbronchial, intramuscular or oral administration. The dosage and method of administration vary according to the body-weight and age of a patient and the administration method; however, one skilled in the art can routinely select a suitable method of administration. If said compound is encodable by a DNA, the DNA can be inserted into a vector for gene therapy and the vector administered to a patient to perform the therapy. The dosage and method of administration vary according to the body-weight, age, and symptoms of the patient; however, one skilled in the art can suitably select them.

For example, although the dose of a compound that binds to a protein of the present invention and regulates its activity depends on the symptoms, the dose is generally about 0.1 mg to about 100 mg per day, preferably about 1.0 mg to about 50 mg per day and more preferably about 1.0 mg to about 20 mg per day, when administered orally to a normal adult human (weight 60 kg).

When administering the compound parenterally, in the form of an injection to a normal adult human (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. In the case of other animals, the appropriate dosage amount may be routinely calculated by converting to 60 kgs of body-weight.

Assessing the Prognosis of a Subject with Breast Cancer:

The present invention also provides a method of assessing the prognosis of a subject with BRC including the step of comparing the expression of one or more BRC-associated genes in a test cell population to the expression of the BRC-associated genes A5657, B9769 and C7965 in a reference cell population derived from patients over a spectrum of disease stages. By comparing the gene expression of one or more BRC-associated genes in the test cell population and the reference cell population(s), or by comparing the pattern of gene expression over time in test cell populations derived from the subject, the prognosis of the subject can be assessed.

For example, an increase in the expression of one or more of the BRC-associated genes A5657, B9769 and C7965 as compared to a normal control indicates less favorable prognosis. Conversely, a similarity to the expression of one or more of the BRC-associated genes A5657, B9769 and C7965 as compared to normal control indicates a more favorable prognosis for the subject. Preferably, the prognosis of a subject can be assessed by comparing the expression profile of gene selected form group consisting of the BRC-associated genes A5657, B9769 and C7965. The classification score (CS) may be use for the comparing the expression profile.

Kits:

The present invention also includes a BRC-detection reagent, e.g., a nucleic acid that specifically binds to or identifies one or more BRC nucleic acids, such as oligonucleotide sequences which are complementary to a portion of a BRC nucleic acid, or an antibody that bind to one or more proteins encoded by a BRC nucleic acid. The detection reagents may be packaged together in the form of a kit. For example, the detection reagents may be packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay may also be included in the kit. The assay format of the kit may be a Northern hybridization or a sandwich ELISA, both of which are known in the art.

For example, a BRC detection reagent may be immobilized on a solid matrix, such as a porous strip, to form at least one BRC detection site. The measurement or detection region of the porous strip may include a plurality of sites, each containing a nucleic acid. A test strip may also contain sites for negative and/or positive controls. Alternatively, control sites may be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of BRC present in the sample. The detection sites may be configured in any suitably detectable shape and are typically in the shape of a bar or dot spanning the width of a test strip.

Alternatively, the kit may contain a nucleic acid substrate array comprising one or more nucleic acids. The nucleic acids on the array specifically identify one or more nucleic acid sequences represented by the BRC-associated genes A5657, B9769 and C7965. The expression of 2 or more of the nucleic acids represented by the BRC-associated genes A5657, B9769 and C7965 may be identified by virtue of the level of binding to an array test strip or chip. The substrate array can be on, e.g., a solid substrate, such as a “chip” described in U.S. Pat. No. 5,744,305, the contents of which are incorporated by reference herein in its entirety.

Arrays and Pluralities:

The present invention also includes a nucleic acid substrate array comprising one or more nucleic acids. The nucleic acids on the array specifically correspond to one or more nucleic acid sequences represented by the BRC-associated genes A5657, B9769 and C7965. The level of expression of 2 or more of the nucleic acids represented by the BRC-associated genes A5657, B9769 and C7965 may be identified by detecting nucleic acid binding to the array.

The present invention also includes an isolated plurality (i.e., a mixture of two or more nucleic acids) of nucleic acids. The nucleic acids may be in a liquid phase or a solid phase, e.g., immobilized on a solid support such as a nitrocellulose membrane. The plurality includes one or more of the nucleic acids represented by the BRC-associated genes A5657, B9769 and C7965. In various embodiments, the plurality includes 2 or more of the nucleic acids represented by the BRC-associated genes listed in A5657, B9769 and C7965.

Methods of Inhibiting Breast Cancer:

The present invention further provides a method for treating or alleviating a symptom of BRC in a subject by decreasing the expression of one or more of the up-regulated BRC-associated genes A5657, B9769 and C7965 (or the activity of its gene product). Suitable therapeutic compounds can be administered prophylactically or therapeutically to a subject suffering from or at risk of (or susceptible to) developing BRC. Such subjects can be identified using standard clinical methods or by detecting an aberrant level of expression of one or more BRC-associated genes A5657, B9769 and C7965 or aberrant activity of its gene product. In the context of the present invention, suitable therapeutic agents include, for example, inhibitors of cell cycle regulation, cell proliferation, and protein kinase activity.

The therapeutic method of the present invention may include the step of decreasing the expression, function, or both, of one or more gene products of genes whose expression is aberrantly increased (“up-regulated” or “over-expressed” gene) in breast cells. Expression may be inhibited in any of several ways known in the art. For example, expression can be inhibited by administering to the subject a nucleic acid that inhibits, or antagonizes, the expression of the over-expressed gene or genes, e.g., an antisense oligonucleotide or small interfering RNA which disrupts expression of the over-expressed gene or genes.

Antisense Nucleic Acids:

As noted above, antisense nucleic acids corresponding to the nucleotide sequence of the BRC-associated genes A5657, B9769 and C7965 can be used to reduce the expression level of the genes. Antisense nucleic acids corresponding to the BRC-associated genes A5657, B9769 and C7965 that are up-regulated in breast cancer are useful for the treatment of breast cancer. Specifically, the antisense nucleic acids of the present invention may act by binding to the BRC-associated genes A5657, B9769 and C7965, or mRNAs corresponding thereto, thereby inhibiting the transcription or translation of the genes, promoting the degradation of the mRNAs, and/or inhibiting the expression of proteins encoded by the BRC-associated genes A5657, B9769 and C7965, thereby, inhibiting the function of the proteins. The term “antisense nucleic acids” as used herein encompasses both nucleotides that are entirely complementary to the target sequence and those having a mismatch of one or more nucleotides, so long as the antisense nucleic acids can specifically hybridize to the target sequences. For example, the antisense nucleic acids of the present invention include polynucleotides that have a homology of at least 70% or higher, preferably at least 80% or higher, more preferably at least 90% or higher, even more preferably at least 95% or higher over a span of at least 15 continuous nucleotides. Algorithms known in the art can be used to determine the homology.

The antisense nucleic acid of the present invention act on cells producing the proteins encoded by BRC-associated marker genes A5657, B9769 and C7965 by binding to the DNAs or mRNAs encoding the proteins, inhibiting their transcription or translation, promoting the degradation of the mRNAs, and inhibiting the expression of the proteins, thereby resulting in the inhibition of the protein function.

An antisense nucleic acid of the present invention can be made into an external preparation, such as a liniment or a poultice, by admixing it with a suitable base material which is inactive against the nucleic acid.

Also, as needed, the antisense nucleic acids of the present invention can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids of the present invention can be given to the patient by direct application onto the ailing site or by injection into a blood vessel so that it will reach the site of ailment. An antisense-mounting medium can also be used to increase durability and membrane-permeability. Examples include, but are not limited to, liposomes, poly-L-lysine, lipids, cholesterol, lipofectin or derivatives of these.

The dosage of the antisense nucleic acid of the present invention can be adjusted suitably according to the patient's condition and used in desired amounts. For example, a dose range of 0.1 to 100 mg/kg, preferably 0.1 to 50 mg/kg can be administered.

The antisense nucleic acids of the present invention inhibit the expression of a protein of the present invention and are thereby useful for suppressing the biological activity of the protein of the invention. In addition, expression-inhibitors, comprising antisense nucleic acids of the present invention, are useful in that they can inhibit the biological activity of a protein of the present invention.

The method of the present invention can be used to alter the expression in a cell of an up-regulated BRC-associated gene, e.g., up-regulation resulting from the malignant transformation of the cells. Binding of the siRNA to a transcript corresponding to one of the BRC-associated genes A5657, B9769 or C7965 in the target cell results in a reduction in the protein production by the cell. The length of the oligonucleotide is at least 10 nucleotides and may be as long as the naturally-occurring transcript. Preferably, the oligonucleotide is 19-25 nucleotides in length. Most preferably, the oligonucleotide is less than 75, 50, 25 nucleotides in length.

The antisense nucleic acids of present invention include modified oligonucleotides. For example, thioated oligonucleotides may be used to confer nuclease resistance to an oligonucleotide.

Also, an siRNA against a marker gene can be used to reduce the expression level of the marker gene. Herein, term “siRNA” refers to a double stranded RNA molecule which prevents translation of a target mRNA. Standard techniques for introducing siRNA into the cell may be used, including those in which DNA is a template from which RNA is transcribed. In the context of the present invention, the siRNA comprises a sense nucleic acid sequence and an anti-sense nucleic acid sequence against an up-regulated marker gene, such as the BRC-associated genes A5657, B9769 and C7965. The siRNA is constructed such that a single transcript has both the sense and complementary antisense sequences from the target gene, e.g., a hairpin.

siRNA of A5657, B9769 or C7965, which hybridize to target mRNA, decrease or inhibit production of the A5657, B9769 and C7965 polypeptides encoded by the A5657, B9769 and C7965 genes, respectively, by associating with the normally single-stranded mRNA transcript, thereby interfering with translation and thus, expression of the protein. The siRNA is preferably less than 500, 200, 100, 50, or 25 nucleotides in length. More preferably the siRNA is 19-25 nucleotides in length. Exemplary nucleic acid sequence for the production of A5657, B9769 and C7965 siRNA include the sequences of nucleotides of SEQ ID NOs: 28, 29, 30, 31, 32, 33, and 34 as the target sequence. Furthermore, in order to enhance the inhibition activity of the siRNA, nucleotide “u” can be added to 3′ end of the antisense strand of the target sequence. The number of “u”s to be added is at least 2, generally 2 to 10, preferably 2 to 5. The added “u”s form single strand at the 3′ end of the antisense strand of the siRNA.

An siRNA of A5657, B9769 or C7965 can be directly introduced into the cells in a form that is capable of binding to the mRNA transcripts. Alternatively, a DNA encoding the siRNA of A5657, B9769 or C7965 may be carried in a vector.

Vectors may be produced, for example, by cloning an A5657, B9769 or C7965 target sequence into an expression vector operatively-linked regulatory sequences flanking the A5657, B9769 or C7965 sequence in a manner that allows for expression (by transcription of the DNA molecule) of both strands (Lee, N. S., Dohjima, T., Bauer, G., Li, H., Li, M.-J., Ehsani, A., Salvaterra, P., and Rossi, J. (2002) Expression of small interfering RNAs targeted against HIV-1 rev transcripts in human cells. Nature Biotechnology 20: 500-505.). An RNA molecule that is antisense to A5657, B9769 or C7965 mRNA is transcribed by a first promoter (e.g., a promoter sequence 3′ of the cloned DNA) and an RNA molecule that is the sense strand for the A5657, B9769 or C7965 mRNA is transcribed by a second promoter (e.g., a promoter sequence 5′ of the cloned DNA). The sense and antisense strands hybridize in vivo to generate siRNA constructs for silencing of the A5657, B9769 or C7965 gene. Alternatively, the two constructs can be utilized to create the sense and anti-sense strands of a siRNA construct. Cloned A5657, B9769 or C7965 can encode a construct having secondary structure, e.g., hairpins, wherein a single transcript has both the sense and complementary antisense sequences from the target gene.

A loop sequence consisting of an arbitrary nucleotide sequence can be located between the sense and antisense sequence in order to form the hairpin loop structure. Thus, the present invention also provides siRNA having the general formula 5′-[A]-[B]-[A′]-3′, wherein [A] is a ribonucleotide sequence corresponding to a sequence selected from the group consisting of nucleotides of SEQ ID NOs: 28, 29, 30, 31, 32, 33, and 34,

[B] is a ribonucleotide sequence consisting of 3 to 23 nucleotides, and

[A′] is a ribonucleotide sequence consisting of the complementary sequence of [A]. The region [A] hybridizes to [A′], and then a loop consisting of region [B] is formed. The loop sequence may be preferably 3 to 23 nucleotide in length. The loop sequence, for example, can be selected from group consisting of following sequences (http//www.ambion.com/techlib/tb/tb_{—}506.html). Furthermore, loop sequence consisting of 23 nucleotides also provides active siRNA (Jacque, J.-M., Triques, K., and Stevenson, M. (2002) Modulation of HIV-1 replication by RNA interference. Nature 418: 435-438.).

CCC, CCACC or CCACACC: Jacque, J. M, Triques, K., and Stevenson, M (2002) Modulation of HIV-1 replication by RNA interference. Nature, Vol. 418: 435-438.

UUCG: Lee, N. S., Dohjima, T., Bauer, G., Li, H., Li, M.-J., Ehsani, A., Salvaterra, P., and Rossi, J. (2002) Expression of small interfering RNAs targeted against HIV-1 rev transcripts in human cells. Nature Biotechnology 20: 500-505. Fruscoloni, P., Zamboni, M., and Tocchini-Valentini, G. P. (2003) Exonucleolytic degradation of double-stranded RNA by an activity in Xenopus laevis germinal vesicles. Proc. Natl. Acad. Sci. USA 100(4): 1639-1644.

UUCAAGAGA: Dykxhoorn, D. M., Novina, C. D., and Sharp, P. A. (2002) Killing the messenger: Short RNAs that silence gene expression. Nature Reviews Molecular Cell Biology 4: 457-467.

For example, preferable siRNAs having hairpin loop structure of the present invention are shown below. In the following structure, the loop sequence can be selected from group consisting of, CCC, UUCG, CCACC, CCACACC, and UUCAAGAGA. Preferable loop sequence is UUCAAGAGA (“ttcaagaga” in DNA). Exemplary hairpin siRNA suitable for use in the context of the present invention include:

for A5657-siRNA:

(for target sequence of SEQ ID NO: 28)

caucgcaacuguguugacc-[b]-ggucaacacaguugcgaug;
and

(for target sequence of SEQ ID NO: 29)

ugccagacaguggacagag-[b]-cucuguccacugucuggca.
for B9769-siRNA:

(for target sequence of SEQ ID NO: 30)

gccugcaguuccugcagca-[b]-ugcugcaggaacugcaggc;

(for target sequence of SEQ ID NO: 31)

gcuuccagucugucaaguc-[b]-gacuugacagacuggaagc;
and

(for target sequence of SEQ ID NO: 32)

agcagaggccucuaaugca-[b]-ugcauuagaggccucugcu.
for C7965-siRNA:

(for target sequence of SEQ ID NO: 33)

acugcuccucucagcuucc-[b]-ggaagcugagaggagcagu;
and

(for target sequence of SEQ ID NO: 34)

guacgcuuacuggcaucaa-[b]-uugaugccaguaagcguac.

The nucleotide sequence of suitable siRNAs can be designed using an siRNA design computer program available from the Ambion website (http://www.ambion.com/techlib/misc/siRNA_finder.html). The computer program selects nucleotide sequences for siRNA synthesis based on the following protocol.

Selection of siRNA Target Sites:
1. Beginning with the AUG start codon of the object transcript, scan downstream for AA dinucleotide sequences. Record the occurrence of each AA and the 3′ adjacent 19 nucleotides as potential siRNA target sites. Tuschl, et al. recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with binding of the siRNA endonuclease complex.
2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/.
3. Select qualifying target sequences for synthesis. At Ambion, preferably several target sequences can be selected along the length of the gene to evaluate.

The regulatory sequences flanking the A5657, B9769 or C7965 sequences can be identical or different, such that their expression can be modulated independently, or in a temporal or spatial manner. siRNAs are transcribed intracellularly by cloning the A5657, B9769 or C7965 gene templates, respectively, into a vector containing, e.g., a RNA pol III transcription unit from the small nuclear RNA (snRNA) U6 or the human H1 RNA promoter. For introducing the vector into the cell, transfection-enhancing agent can be used. FuGENE (Rochediagnostices), Lipofectamin 2000 (Invitrogen), Oligofectamin (Invitrogen), and Nucleofactor (Wako pure Chemical) are useful as the transfection-enhancing agent.

The antisense oligonucleotide or siRNA of the present invention inhibits the expression of a polypeptide of the present invention and is thereby useful for suppressing the biological activity of a polypeptide of the invention. Also, expression-inhibitors, comprising the antisense oligonucleotide or siRNA of the invention, are useful in the point that they can inhibit the biological activity of the polypeptide of the invention. Therefore, a composition comprising an antisense oligonucleotide or siRNA of the present invention is useful for treating a breast cancer.

Antibodies:

Alternatively, function of one or more gene products of the genes over-expressed in BRC (e.g., A5657, B9769 or C7965) can be inhibited by administering a compound that binds to or otherwise inhibits the function of the gene products. For example, the compound may be an antibody which binds to the over-expressed gene product or gene products.

The present invention refers above to the use of antibodies, particularly antibodies against a protein encoded by an up-regulated marker gene, or a fragment of such an antibody.

As used herein, the term “antibody” refers to an immunoglobulin molecule having a specific structure, that interacts (i.e., binds) only with the antigen that was used for synthesizing the antibody (i.e., the gene product of an up-regulated marker gene such as A5657, B9769 or C7965) or with an antigen closely related thereto. As noted above, in the context of the present invention, an antibody may be a fragment of an antibody or a modified antibody, so long as it binds to one or more of the proteins encoded by the marker genes. For instance, the antibody fragment may be Fab, F(ab′)₂, Fv, or single chain Fv (scFv), in which Fv fragments from H and L chains are ligated by an appropriate linker (Huston J. S. et al. Proc. Natl. Acad. Sci. U.S.A. 85:5879-5883 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co M. S. et al. J. Immunol. 152:2968-2976 (1994); Better M. and Horwitz A. H. Methods Enzymol. 178:476-496 (1989); Pluckthun A. and Skerra A. Methods Enzymol. 178:497-515 (1989); Lamoyi E. Methods Enzymol. 121:652-663 (1986); Rousseaux J. et al. Methods Enzymol. 121:663-669 (1986); Bird R. E. and Walker B. W. Trends Biotechnol. 9:132-137 (1991)).

An antibody may be modified by conjugation with a variety of molecules, such as polyethylene glycol (PEG). The present invention provides such modified antibodies. The modified antibody can be obtained by chemically modifying an antibody. Such modification methods are conventional in the field.

Alternatively, an antibody may comprise a chimeric antibody having a variable region derived from a nonhuman antibody and a constant region derived from a human antibody, or a humanized antibody, comprising a complementarity determining region (CDR) derived from a nonhuman antibody, a frame work region (FR) and a constant region derived from a human antibody. Such antibodies can be prepared by using known technologies.

Cancer therapies directed at specific molecular alterations that occur in cancer cells have been validated through clinical development and regulatory approval of anti-cancer drugs such as trastuzumab (Herceptin) for the treatment of advanced breast cancer, imatinib methylate (Gleevec) for chronic myeloid leukemia, gefitinib (Iressa) for non-small cell lung cancer (NSCLC), and rituximab (anti-CD20 mAb) for B-cell lymphoma and mantle cell lymphoma (Ciardiello F, Tortora G. A novel approach in the treatment of cancer: targeting the epidermal growth factor receptor. Clin Cancer Res. 2001 October; 7(10):2958-70. Review.; Slamon D J, Leyland-Jones B, Shak S, Fuchs H, Paton V, Bajamonde A, Fleming T, Eiermann W, Wolter J, Pegram M, Baselga J, Norton L. Use of chemotherapy plus a monoclonal antibody against HER2 for metastatic breast cancer that overexpresses HER2. N Engl J. Med. 2001 Mar. 15; 344(11):783-92.; Rehwald U, Schulz H, Reiser M, Sieber M, Staak J O, Morschhauser F, Driessen C, Rudiger T, Muller-Hermelink K, Diehl V, Engert A. Treatment of relapsed CD20+ Hodgkin lymphoma with the monoclonal antibody rituximab is effective and well tolerated: results of a phase 2 trial of the German Hodgkin Lymphoma Study Group. Blood. 2003 Jan. 15;101(2):420-424.; Fang G, Kim C N, Perkins C L, Ramadevi N. Winton E, Wittmann S and Bhalla K N. (2000). Blood, 96, 2246-2253.). These drugs are clinically effective and better tolerated than traditional anti-cancer agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L. (2002). Oncology, 63 Suppl 1, 47-56.; Klejman A, Rushen L, Morrione A, Slupianek A and Skorski T. (2002). Oncogene, 21, 5868-5876.). Therefore, future cancer treatments will probably involve combining conventional drugs with target-specific agents aimed at different characteristics of tumor cells such as angiogenesis and invasiveness.

These modulatory methods can be performed ex vivo or in vitro (e.g., by culturing the cell with the agent) or, alternatively, in vivo (e.g., by administering the agent to a subject). The methods involve administering a protein or combination of proteins or a nucleic acid molecule or combination of nucleic acid molecules as therapy to counteract aberrant expression of the differentially expressed genes or aberrant activity of their gene products.

Diseases and disorders that are characterized by increased (relative to a subject not suffering from the disease or disorder) expression levels or biological activities of genes and gene products, respectively, may be treated with therapeutics that antagonize (i.e., reduce or inhibit) activity of the over-expressed gene or genes. Therapeutics that antagonize activity can be administered therapeutically or prophylactically.

Accordingly, therapeutics that may be utilized in the context of the present invention include, e.g., (i) a polypeptide of the over-expressed gene or genes, or analogs, derivatives, fragments or homologs thereof; (ii) antibodies to the over-expressed gene or gene products; (iii) nucleic acids encoding the over-expressed gene or genes; (iv) antisense nucleic acids or nucleic acids that are “dysfunctional” (i.e., due to a heterologous insertion within the nucleic acids of one or more over-expressed gene or genes); (v) small interfering RNA (siRNA); or (vi) modulators (i.e., inhibitors, antagonists that alter the interaction between an over-expressed polypeptide and its binding partner). The dysfunctional antisense molecules are utilized to “knockout” endogenous function of a polypeptide by homologous recombination (see, e.g., Capecchi, Science 244: 1288-1292 1989).

Increased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Vaccinating Against Breast Cancer:

The present invention also relates to a method of treating or preventing breast cancer in a subject comprising the step of administering to said subject a vaccine comprising a polypeptide encoded by a nucleic acid selected from the group consisting of the BRC-associated genes A5657, B9769 or C7965, an immunologically active fragment of said polypeptide, or a polynucleotide encoding such a polypeptide or fragment thereof. Administration of the polypeptide induces an anti-tumor immunity in a subject. To induce anti-tumor immunity, a polypeptide encoded by a nucleic acid selected from the group consisting of the BRC-associated genes A5657, B9769 or C7965, an immunologically active fragment of said polypeptide, or a polynucleotide encoding such a polypeptide or fragment thereof is administered to subject in need thereof. The polypeptide or the immunologically active fragments thereof are useful as vaccines against BRC. In some cases, the proteins or fragments thereof may be administered in a form bound to the T cell receptor (TCR) or presented by an antigen presenting cell (APC), such as macrophage, dendritic cell (DC), or B-cells. Due to the strong antigen presenting ability of DC, the use of DC is most preferable among the APCs.

In the present invention, a vaccine against BRC refers to a substance that has the ability to induce anti-tumor immunity upon inoculation into animals. According to the present invention, polypeptides encoded by the BRC-associated genes A5657, B9769 or C7965, or fragments thereof, were suggested to be HLA-A24 or HLA-A*0201 restricted epitopes peptides that may induce potent and specific immune response against BRC cells expressing the BRC-associated genes A5657, B9769 or C7965. Thus, the present invention also encompasses a method of inducing anti-tumor immunity using the polypeptides. In general, anti-tumor immunity includes immune responses such as follows:
induction of cytotoxic lymphocytes against tumors,
induction of antibodies that recognize tumors, and
induction of anti-tumor cytokine production.

Therefore, when a certain protein induces any one of these immune responses upon inoculation into an animal, the protein is determined to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. Specifically, a foreign substance that enters the living body is presented to T cells and B cells by the action of antigen presenting cells (APCs). T cells that respond to the antigen presented by the APCs in an antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to a T cell via an APC, and detecting the induction of CTLs. Furthermore, APCs have the effect of activating CD4+ T cells, CD8+ T cells, macrophages, eosinophils, and NK cells. Since CD4+ T cells and CD8+ T cells are also important in anti-tumor immunity, the anti-tumor immunity-inducing action of the peptide can be evaluated using the activation effect of these cells as indicators.

A method for evaluating the inducing action of CTLs using dendritic cells (DCs) as the APC is well known in the art. DCs are a representative APCs having the strongest CTL-inducing action among APCs. In this method, the test polypeptide is initially contacted with DCs, and then the DCs are contacted with T cells. Detection of T cells having cytotoxic effects against the cells of interest after the contact with DC shows that the test polypeptide has an activity of inducing the cytotoxic T cells. Activity of CTLs against tumors can be detected, for example, using the lysis of ⁵¹Cr-labeled tumor cells as the indicator. Alternatively, the method of evaluating the degree of tumor cell damage using ³H-thymidine uptake activity or LDH (lactose dehydrogenase)-release as the indicator is also well known.

Apart from DCs, peripheral blood mononuclear cells (PBMCs) may also be used as the APC. The induction of CTLs has been reported to be enhanced by culturing PBMCs in the presence of GM-CSF and IL-4. Similarly, CTLs have been shown to be induced by culturing PBMCs in the presence of keyhole limpet hemocyanin (KLH) and IL-7.

Test polypeptides confirmed to possess CTL-inducing activity by these methods are deemed to be polypeptides having DC activation effect and subsequent CTL-inducing activity. Therefore, polypeptides that induce CTLs against tumor cells are useful as vaccines against tumors. Furthermore, APCs that have acquired the ability to induce CTLs against tumors through contact with the polypeptides are also useful as vaccines against tumors. Furthermore, CTLs that have acquired cytotoxicity due to presentation of the polypeptide antigens by APCs can be also be used as vaccines against tumors. Such therapeutic methods for tumors, using anti-tumor immunity due to APCs and CTLs, are referred to as cellular immunotherapy.

Generally, when using a polypeptide for cellular immunotherapy, efficiency of the CTL-induction is known to be increased by combining a plurality of polypeptides having different structures and contacting them with DCs. Therefore, when stimulating DCs with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth of tumor cells is suppressed by those antibodies, the polypeptide is deemed to have the ability to induce anti-tumor immunity.

Anti-tumor immunity is induced by administering the vaccine of this invention, and the induction of anti-tumor immunity enables treatment and prevention of BRC. Therapy against cancer or prevention of the onset of cancer includes any of the following steps, such as inhibition of the growth of cancerous cells, involution of cancer, and suppression of the occurrence of cancer. A decrease in mortality and morbidity of individuals having cancer, decrease in the levels of tumor markers in the blood, alleviation of detectable symptoms accompanying cancer, and such are also included in the therapy or prevention of cancer.

Such therapeutic and preventive effects are preferably statistically significant. For example, in observation, at a significance level of 5% or less, wherein the therapeutic or preventive effect of a vaccine against cell proliferative diseases is compared to a control without vaccine administration. For example, Student's t-test, the Mann-Whitney U-test, or ANOVA may be used for statistical analysis.

The above-mentioned protein having immunological activity or a vector encoding the protein may be combined with an adjuvant. An adjuvant refers to a compound that enhances the immune response against the protein when administered together (or successively) with the protein having immunological activity. Exemplary adjuvants include, but are not limited to, cholera toxin, salmonella toxin, alum, and such, but are not limited thereto. Furthermore, the vaccine of this invention may be combined appropriately with a pharmaceutically acceptable carrier. Examples of such carriers include sterilized water, physiological saline, phosphate buffer, culture fluid, and such. Furthermore, the vaccine may contain as necessary, stabilizers, suspensions, preservatives, surfactants, and such. The vaccine can be administered systemically or locally. Vaccine administration can be performed by single administration, or boosted by multiple administrations.

When using an APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and following the induction of APCs or CTLs, the cells may be administered to the subject. APCs can be also induced by introducing a vector encoding the polypeptide into PBMCs ex vivo. APCs or CTLs induced in vitro can be cloned prior to administration. By cloning and growing cells having high activity of damaging target cells, cellular immunotherapy can be performed more effectively. Furthermore, APCs and CTLs isolated in this manner may be used for cellular immunotherapy not only against individuals from whom the cells are derived, but also against similar types of tumors from other individuals.

Furthermore, a pharmaceutical composition for treating or preventing a cell proliferative disease, such as cancer, comprising a pharmaceutically effective amount of the polypeptide of the present invention is provided. The pharmaceutical composition may be used for raising anti tumor immunity.

Pharmaceutical Compositions for Inhibiting BRC or Malignant BRC:

In the context of the present invention, suitable pharmaceutical formulations include those suitable for oral, rectal, nasal, topical (including buccal and sub-lingual), vaginal or parenteral (including intramuscular, subcutaneous and intravenous) administration, or for administration by inhalation or insufflation. Preferably, administration is intravenous. The formulations are optionally packaged in discrete dosage units.

Pharmaceutical formulations suitable for oral administration include capsules, cachets or tablets, each containing a predetermined amount of active ingredient. Suitable formulations also include powders, granules, solutions, suspensions and emulsions. The active ingredient is optionally administered as a bolus electuary or paste. Tablets and capsules for oral administration may contain conventional excipients, such as binding agents, fillers, lubricants, disintegrant and/or wetting agents. A tablet may be made by compression or molding, optionally with one or more formulational ingredients. Compressed tablets may be prepared by compressing in a suitable machine the active ingredients in a free-flowing form, such as a powder or granules, optionally mixed with a binder, lubricant, inert diluent, lubricating, surface active and/or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives, such as suspending agents, emulsifying agents, non-aqueous vehicles (which may include edible oils), and/or preservatives. The tablets may optionally be formulated so as to provide slow or controlled release of the active ingredient therein. A package of tablets may contain one tablet to be taken on each of the month.

Formulations suitable for parenteral administration include aqueous and non-aqueous sterile injection solutions, optionally contain anti-oxidants, buffers, bacteriostats and solutes which render the formulation isotonic with the blood of the intended recipient; as well as aqueous and non-aqueous sterile suspensions including suspending agents and/or thickening agents. The formulations may be presented in unit dose or multi-dose containers, for example as sealed ampoules and vials, and may be stored in a freeze-dried (lyophilized) condition, requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations suitable for rectal administration include suppositories with standard carriers such as cocoa butter or polyethylene glycol. Formulations suitable for topical administration in the mouth, for example, buccally or sublingually, include lozenges, containing the active ingredient in a flavored base such as sucrose and acacia or tragacanth, and pastilles, comprising the active ingredient in a base such as gelatin and glycerin or sucrose and acacia. For intra-nasal administration, the compounds of the invention may be used as a liquid spray, a dispersible powder, or in the form of drops. Drops may be formulated with an aqueous or non-aqueous base also comprising one or more dispersing agents, solubilizing agents and/or suspending agents.

For administration by inhalation the compounds can be conveniently delivered from an insufflator, nebulizer, pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base, such as lactose or starch. The powder composition may be presented in unit dosage form, for example, as capsules, cartridges, gelatin or blister packs, from which the powder may be administered with the aid of an inhalator or insufflators.

Other formulations include implantable devices and adhesive patches which release a therapeutic agent.

When desired, the above described formulations, adapted to give sustained release of the active ingredient, may be employed. The pharmaceutical compositions may also contain other active ingredients, such as antimicrobial agents, immunosuppressants and/or preservatives.

It should be understood that in addition to the ingredients particularly mentioned above, the formulations of this invention may include other agents conventional in the art with regard to the type of formulation in question. For example, formulations suitable for oral administration may include flavoring agents.

Preferred unit dosage formulations contain an effective dose, as recited below, or an appropriate fraction thereof, of the active ingredient.

For each of the aforementioned conditions, the compositions, e.g., polypeptides and organic compounds, can be administered orally or via injection at a dose ranging from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age and sex of the subject, the precise disorder being treated, and its severity. Also the route of administration may vary depending upon the condition and its severity. In any event, appropriate and optimum dosages may be routinely calculated by those skilled in the art, taking into consideration the above-mentioned factors.

Aspects of the present invention are described in the following examples, which are not intended to limit the scope of the invention described in the claims. The following examples illustrate the identification and characterization of genes differentially expressed in BRC cells.

### EXAMPLES

Tissue obtained from diseased tissue (e.g., epithelial cells from BRC) and normal tissues was evaluated to identity genes which are differently expressed or a disease state, e.g., BRC. The assays were carried out as follows.

Patients and Tissue Samples:

Primary breast cancers were obtained with informed consent from 81 patients (12 ductal carcinoma in situ and 69 invasive ductal carcinoma from 2 cm to 5 cm(T2), median age 45 in a range of 21 to 68 years old) who treated at Department of Breast Surgery, Cancer Institute Hospital, Tokyo, Japan, concerning which all patients had given informed consent (Table 2). Clinical information was obtained from medical records and each tumor was diagnosed according to histopathological subtype and grade by pathologists. Tumor tissue was used to evaluate tumor type (according to the World Health Organization classification and the Japanese cancer society classification). Clinical stage was judged according to the JBCS TNM classification. No significant differences were observed between node-positive and node-negative cases. The presence of angioinvasive growth and extensive lymphocytic infiltrate was determined by pathologists. Estrogen receptor (ER) and progesterone receptor (PgR) expression was determined by EIA (ER negative when less than 13 fmol/mg protein, BML). A mixture of normal breast ductal cells from the 15 premenopausal patients with breast cancer or the 12 postmenopausal patients were used as normal controls, respectively. All samples were immediately frozen and stored at −80° C.

Tissue Samples and LMM:

Clinical and pathological information on the tumor is detailed in Table 2. Samples were embedded in TissueTek OCT medium (Sakura) and then stored at −80° C. until use. Frozen specimens were serially sectioned in 8-μm slices with a cryostat and stained with hematoxylin and eosin to define the analyzed regions. To avoid cross-contamination of cancer and noncancerous cells, these two populations were prepared by EZ Cut LMM System (SL Microtest GmbH) followed the manufacture's protocol with several modifications. To minimize the effects during storage process and tissue collection, the cancer tissues were carefully handled by the same procedure. To check the quality of RNAs, total RNA extracted from the residual tissue of each case were electrophoresed under the degenerative agarose gel, and confirmed their quality by a presence of ribosomal RNA bands.

Cell Lines:

Human-breast cancer cell lines HBC4, HBC5, MDA-MB-231, BSY-1 were kindly provided by Dr. Yamori (The Japanese Foundation of Cancer Research, Tokyo), MCF7, T47D, SKBR3, HCC1937, MDA-MB-435S, YMB1, HBL100, COS7, NIH3T3 were obtained from ATCC. All cells were cultured in appropriate media; i.e. RPMI-1640 (Sigma, St. Louis, Mo.) for HBC4, HBC5, SKBR3, T47D, YMB1, and HCC1937 (with 2 mM L-glutamine); Dulbecco's modified Eagle's medium (Invitrogen, Carlsbad, Calif.) for HBL100, COS7, NIH3T3; EMEM (Sigma) with 0.1mM essential amino acid (Roche), 1 mM sodium pyruvate (Roche), 0.01 mg/ml Insulin(Sigma) for MCM7; L-15 (Roche) for MDA-MB-231 and MDA-MB-435S. Each medium was supplemented with 10% fetal bovine serum (Cansera) and 1% antibiotic/antimycotic solution (Sigma). MDA-MB-231 and MDA-MB-435S cells were maintained at 37° C. an atmosphere of humidified air without CO₂. Other cell lines were maintained at 37° C. an atmosphere of humidified air with 5% CO₂.

RNA Extraction and 17-Based RNA Amplification:

Total RNA was extracted from each population of laser captured cells into 35011 RLT lysis buffer (QIAGEN). The extracted RNA was treated for 30 minutes at room temperature with 30 units of DNase I (QIAGEN). After inactivation at 70° C. for 10 min, the RNAs were purified with an RNeasy Mini Kit (QIAGEN) according to the manufacturer's recommendations. All of the DNase I treated RNA was subjected to T7-based amplification using Ampliscribe T7 Transcription Kit (Epicentre Technologies). Two rounds of amplification yielded 28.8-329.4 μg of amplified RNAs (aRNAs) for each sample, whereas when RNAs from normal samples from 15 premenopausal patients or 12 postmenopausal patients were amplified, total of 2240.2 μg and 2023.8 μg were yielded, respectively. 2.5 μg aliquots of aRNA from each cancerous cells and noncancerous breast ductal cells were reverse-transcribed in the presence of Cy5-dCTP and Cy3-dCTP (Amersham Biosciences), respectively.

cDNA Microarrays:

A “genome-wide” cDNA microarray system was established containing 23,040 cDNAs selected from the UniGene database (build #131) the National Center for Biotechnology Information (NCBI). Fabrication of the cDNA microarray slides has been described elsewhere (Ono K, Tanaka T, Tsunoda T, Kitahara O, Kihara C, Okamoto A, Ochiai K, Katagiri T and Nakamura Y. Identification by cDNA Microarray of Genes Involved in Ovarian Carcinogenesis. Cancer Res., 60, 5007-11, 2000.). Briefly, the cDNAs were amplified by reverse transcription-PCR using poly(A)+RNA isolated from various human organs as templates; lengths of the amplicons ranged from 200 to 1100 bp without repetitive or poly(A) sequences. The PCR products were spotted in duplicate on type-7 glass slides (Amersham Bioscience) using a Lucidea Array Spotter (Amersham Biosciences); 4,608 or 9,216 genes were spotted in duplicate on a single slide. Three different sets of slides (total 23,040 genes) were prepared, on each of which the same 52 housekeeping genes and two kinds of negative-control genes were spotted as well.

Hybridization and Acquisition of Data:

Hybridization and washing were performed according to protocols described previously except that all processes were carried out with an Automated Slide Processor (Amersham Biosciences) (Giuliani, N., et al., V. Human myeloma cells stimulate the receptor activator of nuclear factor-kappa B ligand (RANKL) in T lymphocytes: a potential role in multiple myeloma bone disease. Blood, 100: 4615-4621, 2002.). The intensity of each hybridization signal was calculated photometrically by the ArrayVision computer program (Amersham Biosciences) and background intensity was subtracted. The fluorescence intensities of Cy5 (tumor) and Cy3 (control) for each target spot were adjusted so that the mean Cy5/Cy3 ratio was performed using averaged signals from the 52 housekeeping genes. Because data derived from low signal intensities are less reliable, a cut-off value on each slide was determined as described previously (Ono, K., et al., Identification by cDNA microarray of genes involved in ovarian carcinogenesis. Cancer Res, 60: 5007-5011, 2000.) excluded genes from further analysis when both Cy3 and Cy5 dyes yielded signal intensities lower than the cut-off (Saito-Hisaminato, A., Katagiri, T., Kakiuchi, S., Nakamura, T., Tsunoda, T., and Nakamura, Y. Genome-wide profiling of gene expression in 29 normal human tissues with a cDNA microarray. DNA Res, 9: 3545, 2002.). For other genes, the Cy5/Cy3 ratio was calculated using the raw data of each sample.

Identification and Isolation of a Novel Human Genes A5657, B9769 and C7965:

Total RNAs were extracted and amplified as discussed above. Aliquots of amplified RNA from breast cancer cells and the normal breast ductal cells were labeled by reverse transcription with Cy5-dCTP and Cy3-dCTP, respectively (Amersham Biosciences, Buckinghamshire, UK). Hybridization, washing, and detection were carried out as described above. To detect genes that were commonly up-regulated in breast cancer, the overall expression patterns of the 23,040 genes on the microarray were screened to select those with expression ratios >3.0 that were present in >50% of i) all of 81 breast cancer cases, ii) 69 invasive ductal carcinomas, iii) 31 well-, iv) 14 moderately, or v) 24 poorly-differentiated lesions, respectively. Among the total of 102 genes that appeared to up-regulated in tumor cells, the three with the following in-house identification number, A5657, B9769 and C7965 were selected for further examination because their expression ratio were greater than 3.0 in more than 50% of the informative breast cancer cases.

Semi-Quantitative RT-PCR:

The three up-regulated genes, A5657, B9769 and C7965, mentioned above were selected and their expression levels were examined by applying the semi-quantitative RT-PCR experiments. Specifically, total RNA were extracted, amplified and reverse transcribed as described above. Appropriate dilutions of each single-stranded cDNA were prepared for subsequent PCR amplification by monitoring the glyceraldehyde-3-phosphate dehydrogenase (GAPDB) as a quantitative internal control. The PCR primer sequences were

| | |
|---|---|
| 5′-CGACCACTTTGTCAAGCTCA-3′ | (SEQ ID No. 7) |
| and | |
| 5′-GGTTGAGCACAGGGTACTTTATT-3′ | (SEQ ID No. 8) |
| for GAPDH; | |
| 5′-CAAATATTAGGTGGAGCCAACAC-3′ | (SEQ ID No. 9) |
| and | |
| 5′-TAGATCACCTTGGCAAAGAACAC-3′ | (SEQ ID No. 10) |
| for A5657, | |
| 5′-ACCTCAAGTCCCTCCTGGAA-3′ | (SEQ ID No. 11) |
| and | |
| 5′-TCAGTTTCAACAGGTAAGGCGAT-3′ | (SEQ ID No. 12) |
| for B9769, | |
| 5′-AGAGCCATAGAAACTGCTCCTCT-3′ | (SEQ ID No. 13) |
| and | |
| 5′-CATAACTGCATAGACAGCACGTC-3′ | (SEQ ID No. 14) |
| for C7965. | |

Northern-Blot Analysis:

Total RNAs were extracted from all breast cancer cell lines using RNeasy kit (QIAGEN) according to the manufacturer's instructions. After treatment with DNase I (Nippon Gene, Osaka, Japan), mRNA was isolated with mRNA purification kit (Amersham Biosciences) following the manufacturer's instructions. A 1-μg aliquot of each mRNA, along with polyA(+) RNAs isolated from normal adult human breast (Biochain), lung, heart, liver, kidney, bone marrow (BD, Clontech, Palo Alto, Calif.), were separated on 1% denaturing agarose gels and transferred to nylon membranes (Breast cancer-Northern blots). Breast cancer- and Human multiple-tissue Northern blots (Clontech, Palo Alto, Calif.) were hybridized with an [α³²P]-dCTP-labeled PCR products of A5657, B9769 and C7965 prepared by RT-PCR (see below). Pre-hybridization, hybridization and washing were performed according to the supplier's recommendations. The blots were autoradiographed with intensifying screens at −80° C. for 14 days. Specific probes for A5657 (541 bp), B9769 (499 bp) and C7965 (238 bp) were prepared by PCR using a primer set as mentioned in semi-quantitative RT-PCR section;

| | |
|---|---|
| 5′-CAAATATTAGGTGGAGCCAACAC-3′ | (SEQ ID No. 9) |
| and | |
| 5′-TAGATCACCTTGGCAAAGAACAC-3′ | (SEQ ID No. 10) |
| for A5657, | |
| 5′-ACCTCAAGTCCCTCCTGGAA-3′ | (SEQ ID No. 11) |
| and | |
| 5′-TCAGTTTCAACAGGTAAGGCGAT-3′ | (SEQ ID No. 12) |
| for B9769, | |
| 5′-GGGAAGAGAAGTCCCGAGTC-3′ | (SEQ ID No. 15) |
| and | |
| 5′-TCCTTATTCTGAATTTCCAGAATC-3′ | (SEQ ID No. 16) |
| for C7965. | |

Rapid Amplification cDNA End (RACE)-PCR:

To detect the full length of transcripts of C7965, 5′ RACE was performed with a Marathon cDNA amplification kit (BD, CLONTECH) following the manufacturer's instruction. When 1^{st} PCR was performed with reverse primers (5′-CAAGCAGTCCTACCAGGGTTCGGAAGCTGA-3′) (SEQ ID No.17) using cDNA prepared from the breast cancer cell line, MCF7, as a template, multiple product bands were detected. 1^{st} 5′ RACE-PCR was performed under the following conditions: initial denaturation at 94° C. for 1 min; 35 cycles of 94° C. for 30 sec; 68° C. for 2 min; followed by a final elongation at 68° C. for 7 min. After the 1^{st}-PCR products were diluted, strong product bands were detected by Nested PCR with nested reverse primers (5′-CCAGGGTTCGGAAGCTGAGAGGAGCAGTTT-3′) (SEQ ID No.18). Nested-PCR was performed under the following conditions: initial denaturation at 94° C. for 1 min; 2 cycles of 94° C. for 30 sec, 72° C. for 2 min, and 2 cycles of 94° C. for 30 sec, 70° C. for 2 min, 15 cycles of 94° C. for 30 sec, 68° C. for 2 min; followed by a final elongation at 68° C. for 7 min. Sequences were identified using Gel extraction kit (Qiagen) and TOPO TA cloning kit (Invitrogen), following the manufacturer's instruction.

Construction of Expression Vectors:

The entire coding sequences of A5657, B9769 and C7965 cDNA were amplified by PCR using KOD-Plus DNA polymerase (Toyobo, Osaka, Japan) with primers

| A5657 forward; | |
|---|---|
| 5′-CCGGAATTCATGCAGAGAGCTTCACGTCTG- | (SEQ ID No. 19) |
| 3′ | |
| and | |
| A5657 reverse; | |
| 5′-CCGCTCGAGAACATCAGGATGAAATTTCTTT | (SEQ ID No. 20) |
| TC-3′, | |
| primers B9769-forward; | |
| 5′-CCGGAATTCATGAGCGGTGCGGGGGTGGCG- | (SEQ ID No. 21) |
| 3′ | |
| and | |
| B9769-reverse; | |
| 5′-CCGCTCGAGAAGCACTGAGCGATGCAGGCG- | (SEQ ID No. 22) |
| 3′ | |
| and | |
| primers C7965-forward; | |
| 5′-CCGGAATTCATGGACGCAGAGCTGGCAGAGG | (SEQ ID No. 23) |
| TGCG-3′ | |
| and | |
| C7965-reverse; | |
| 5′-CCGCTCGAGGTTGTTCTCCTCTGCACAAAG- | (SEQ ID No. 24) |
| 3′. | |

The PCR products were inserted into the EcoRI and XhoI sites of pCAGGS-3×FLAGn and pCAGGSnHA, pcDNA3.1(+)-Myc/H is (Invitrogen) expression vectors, respectively. For ubiquitin binding assay, the HA-ubiquitin expression vector (pcdef3-HA-RPS27A) was a gift from Dr. Kohei Miyazono (The Cancer Institute of Japanese Foundation for Cancer Research). These constructs (PCAGGS-A5657-HA, pcDNA3.1-A5657-Myc/H is, pCAGGS-3×FLAG-A5657, and pcdef3-HA-RPPS27A(Ub80a)) were confirmed by DNA sequencing. For cell growth promoting assay, the entire coding sequence of mutant H-ras (G12K) was constructed by Dr. Motoko Unoki.
Immunocytochemical Staining:

To examine the sub-cellular localization of the A5657, B9769 and C7965 proteins, T47D cells were seeded at 5×10⁴ cells per well for A5657 and B9769, and COS7 cells at 1×10⁴ (low density) and 1×10⁵ (high density) for B9769 and C7965. After 24 hours, the cells were transiently transfected with 1 μg of pCAGGS-A5657-HA into T47D cells using FuGENE 6 transfection reagent (Roche) according to the manufacturer's instructions, respectively. Then, the cells were fixed with PBS containing 4% paraformaldehyde for 15 min, and rendered permeable with PBS containing 0.1% Triton X-100 for 2.5 min at 4° C. Subsequently the cells were covered with 3% BSA in PBS for 12 hours at 4° C. to block non-specific hybridization. Next, A5657-HA-transfected T47D cells were incubated with a mouse anti-HA antibody (SANTA CRUZ) at 1: 1000 dilution or a mouse anti-myc antibody (Sigma) at 1:1000 dilution, respectively. After washing with PBS, both transfected-cells were stained by an Alexas94-conjugated anti-mouse secondary antibody (Molecular Probe) at 1:5000 dilution. Moreover, the sub-cellular localization between B9769 and other cytoskelton proteins was compared. After transfection with 1 μg of pcDNA3.1(+)-B9769-myc-his, cells were with a rabbit anti-myc antibody (SANTACRUZ) at 1:1000 dilution or a mouse anti-β tubulin antibody(SIGMA) at 1:500 dilution. After washing with PBS, transfected-T47D cells were stained by an Alexa488-conjugated anti-rabbit secondary antibody (Molecular Probe), an Alexa594-conjugated anti-mouse secondary antibody (Molecular Probe) at 1:5000 dilution, Alexa594 conjugated Phalloidin at 1:50 dilution. Furthermore, to examine sub-cellular localization of B9769 in low density or high density of cells, COS7 cells were transfected with 1 μg of pCAGGSn3F-B9769-HA, and performed immunocytochemical staining with the same procedures as above mentioned. Transfected cells were incubated with a mouse anti-HA antibody (SANTACRUZ) at 1:1000 dilution. After washing with PBS, COS7 cells were stained by an Alexas94-conjugated anti-mouse secondary antibody (Molecular Probe) at 1:5000 dilution. Nuclei were counter-stained with 4′,6′-diamidine-2′-phenylindole dihydrochloride (DAPI). Furthermore, to examine sub-cellular localization of C7965 in low density or high density of cells, COS7 cells were transfected with 1 μg of pcDNA3.1(+)-C7965-Myc/His, and performed immunocytochemical staining with the same procedures as above mentioned. Transfected cells were incubated with a mouse anti-myc antibody (SANTACRUZ) at 1:1000 dilution. After washing with PBS, COS7 cells were stained by an AlexaS94-conjugated anti-mouse secondary antibody (Molecular Probe) at 1:5000 dilution. Nuclei were counter-stained with 4′,6′-diamidine-2′-phenylindole dihydrochloride (DAPI). Fluorescent images were obtained under a TCS SP2 AOBS microscope (Leica, Tokyo, Japan). Construction of A5657. B9769 and C7965 specific-siRNA expression vector using psi U6X3.0:

A vector-based RNAi system was established using a psiU6BX siRNA expression vector described in the literature (Shimokawa T., Furukawa Y., Sakai M., Li M., Miwa N., Lin Y. M. Nakamura Y. Involvement of the FGF18 Gene in Colorectal Carcinogenesis, as a Novel Downstream Target of the β-Catenin/T-Cell Factor Complex63, Cancer Res., 63, 6116-20,2003). An siRNA expression vector against A5657 (psiU6BX-A5657), B9769 (psiU6BX-B9769) and C7965 (psiU6BX-C7965) were prepared by cloning of double-stranded oligonucleotides into the BbsI site in the psiU6BX vector. A control plasmid, psiU6BX-EGFP, was prepared by cloning double-stranded oligonucleotides of

TABLE 1

| | |
|---|---|
| 5′-CACCGAAG CAGCACGACTTCTTCTTCAAGA | (SEQ ID No. 25) |
| GAGAAGAAGTCGTGCTGCTTC-3′ | |
| and | |
| 5′-AAAAGAAGCAGCACGACTTCTTCTCTCTTGA | (SEQ ID No. 26) |
| AGAAGAAGTCGTGCTGCTTC-3′ | |
| into the BbsI site in the | |
| psiU6BX3.0 vector. | |

| | |
|---|---|
| target sequense | SEQ ID No. |

| | | |
|---|---|---|
| siEGFP | 5′-GAAGCAGCACGACTTCTT-3′ | 27 |
| A5657 si2 | 5′-CATCGCAACTGTGTTGACC-3′ | 28 |
| A5657 si3 | 5′-TGCCAGACAGTGGACAGAG-3′ | 29 |
| B9769 si1 | 5′-GCCTGCAGTTCCTGCAGCA-3′ | 30 |
| B9769 si2 | 5′-GCTTCCAGTCTGTCAAGTC-3′ | 31 |
| B9769 si4 | 5′-AGCAGAGGCCTCTAATGCA-3′ | 32 |
| C7965 si1 | 5′-ACTGCTCCTCTCAGCTTCC-3′ | 33 |
| C7965 si3 | 5′-GTACGCTTACTGGCATCAA-3′ | 34 |

Gene-Silencing Effect of A5657, B9769 and C7965:

Human breast cancer cells line, T47D, was plated onto 10-cm dishes (1×10⁶ cells/dish) and transfected with psiU6BX-EGFP as negative control, psiU6BX-A5657 using FuGENE6 reagent according to the supplier's recommendations (Roche). Total RNA was extracted from the cells at 7 days after the transfection, and then the knockdown effect of siRNAs was confirmed by semi-quantitative RT-PCR using specific primers for A5657, B9769 and C7965, and for GAPDHas above mentioned. Moreover, transfectants expressing siRNAs using T47D cell lines were grown for 28 days in selective media containing 0.7 mg/ml of neomycin. After fixation with 4% paraformaldehyde, transfected cells were stained with Giemsa solution to assess colony formation. MTT assays were performed to quantify cell viability. After 7 days of culture in the neomycin-containing medium, MTT solution (3-(4,5-dimethylthiazol-2-yl)-2,5-diphenyl tetrazolium bromide) (Sigma) was added at a concentration of 0.5 mg/ml. Following incubation at 37° C. for 2.5 hours, acid-SDS (0.01 N HCl/10% SDS) was added; the suspension was mixed vigorously and then incubated overnight at 37° C. to dissolve the dark blue crystals. Absorbance at 570 nm was measured with a Microplate Reader 550 (BioRad).

Cell Proliferation Assays:

NIH3T3 cells were seeded at 3×10⁶ cells per 150 mm dish. After 24 hours, 16 μg of pcDNA3.1(+)-Myc/His (Invitrogen) as a negative control, pcDNA3.1(+)-A5657-Myc/His, pcDNA3.1(+)-B9769-Myc/H is, pcDNA3.1 (+)-C7965 or pcDNA3.1(+)-H-Ras-mutant-Myc/H is as a positive control were transiently transfected using FuGENE 6 (Roche) into NIH3T3 cells, respectively. Next, 2×10⁴ of NIH3T3 cells per well were re-seeded at 24 hours after transfection. MTT assays were performed at 1, 2, 4 and 6 days after re-seedling as described previously.

Ubiquitin Binding Analysis

COS7 cells were seeded at 5×10⁵ cells per well. After 24 hours, 1 μg of FLAG-tagged A5657 and 0.1 μg of HA-tagged-ubiquitin expression vector (Ub80a-HA) were cotransfected using FuGENE6 (Roche) into COS7 cells. The HA-tagged-ubiquitin expression vector was constructed according to (Imamura et al. and Ebisawa et al (Imamura et al., Nuture 389/6651, 622-6, 1997 “Smad6 inhibits signalling by the TGF-beta super family”; Ebisawa et al., J Biol Chem. 20:276/16, 12477-80, 2001 “Smurfl interacts with transforming growth factor-beta type I receptor through Smad7 and induces receptor degradation”). The cells were added 5 μM proteasome inhibitor, MG132 or 5 μM DMSO at 24 hours after transfection, and 6 hour later, the cells were lysed in 1 ml of 1.0% NP40 buffer (1.0% NP40, 50 mM Tris-HCl (pH 8.0), 150 mM NaCl, and complete protease-inhibitor cocktail). The cell lysates were immunoprecipitated with agarose conjugated with mouse monoclonal anti-FLAG M2-antibody or mouse anti-HA antibody. After six rounds of washing with lysis buffer, the binding proteins were eluted with Flag-M2 or HA peptides (SIGMA). The immunoprecipitates were analysed on Western blots using rabbit anti-HA polyclonal antibody, or rabbit anti-Flag polyclonal antibody. The soluble proteins in sample buffer were loaded on 12% SDS-polyacrylamide gels and transferred to nitrocellulose membranes (Hybond ECL6). The membranes were blocked with BlockAce powder (Yukijirushi, Tokyo, Japan) in Tris-buffered saline containing 0.05% Tween 20 (TBST) and incubated with each antibody for 1 hour at room temperature. The blots were then hybridized with HRP-conjugated secondary antibody (Amersharm Biosciences), and detected using the ECL method (Amersham Biosciences).

Results

Identification of A5657, B9769 and C7965 as Up-Regulated Genes in Breast Cancer Cells:

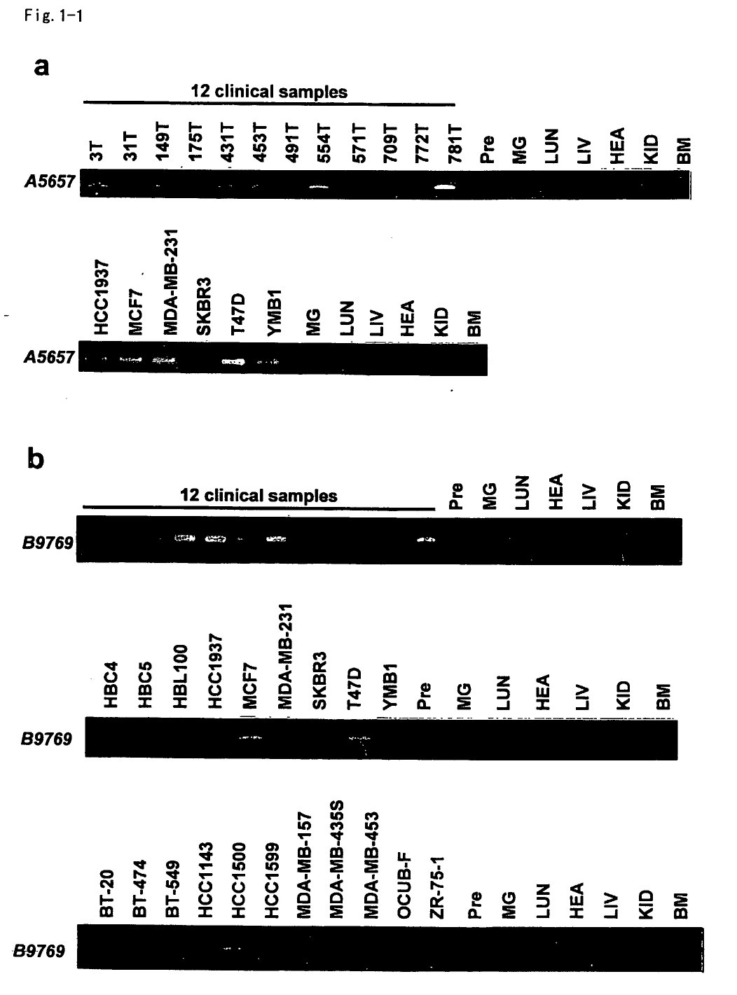

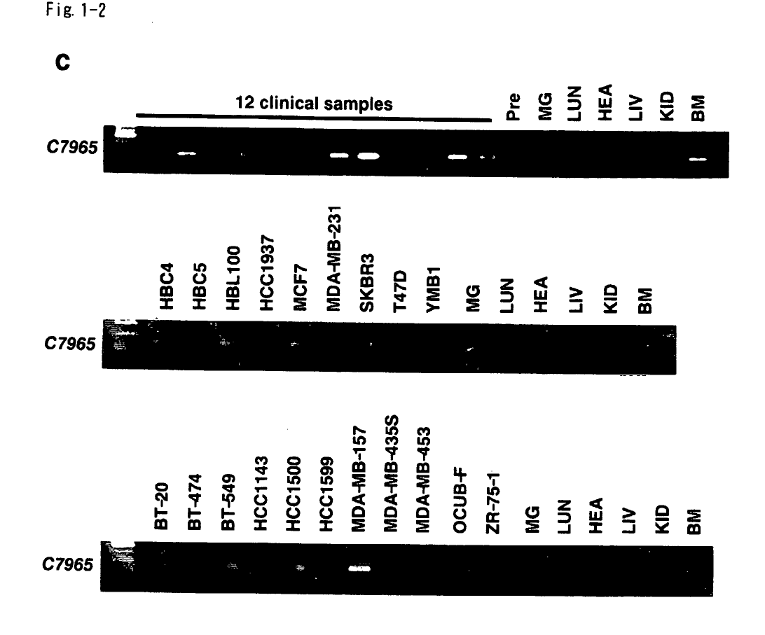

When gene-expression profiles of cancer cells from 81 breast cancer patients were analyzed using a cDNA microarray representing 23,040 human genes, 102 genes that were commonly up-regulated were identified in breast cancer cells. From these up-regulated genes, the genes with in-house code A5657, which designated HSPC150 protein similar to ubiquitin-conjugating enzyme (Genbank Accession NM_{—}014176) (SEQ ID NO: 1), with B9769, which designated hypothetical protein BC016861 (Genbank accession No. NM138770) (SEQ ID NO: 3), and with C7965, which corresponded to EST (SEQ ID NO: 5) were selected. Expression of the genes A5657, B9769 and C7965 were elevated in 38 of 49, 30 of 73 and 28 of 49 breast cancer cells on the microarray in comparison with normal breast ductal cells, respectively. To conform the expression of these up-regulated genes we focused, semi-quantitative RT-PCR analysis was performed to compare the expression of them between breast cancer cells and normal human tissues including normal breast cells. Firstly, it was discovered that A5657 showed the elevated expression in 9 of 12 clinical breast cancer samples (poorly-differentiated type) as compared to normal breast ductal cells and some other normal tissues, and was over-expressed in all of 6 breast cancer cell lines as well (FIG. 1a). Next, is was discovered that B9769 showed the elevated expression in 6 of 12 clinical breast cancer samples (poorly-differentiated type) as compared to normal breast ductal cells, and was over-expressed in 4 of 20 breast cancer cell lines (FIG. 1b). Finally, it was revealed that C7965 showed the elevated expression in 7 of 12 clinical breast cancer samples (well-differentiated type) compared to normal breast ductal cells, and was over-expressed in 15 of 20 breast cancer cell lines FIG. 1c).

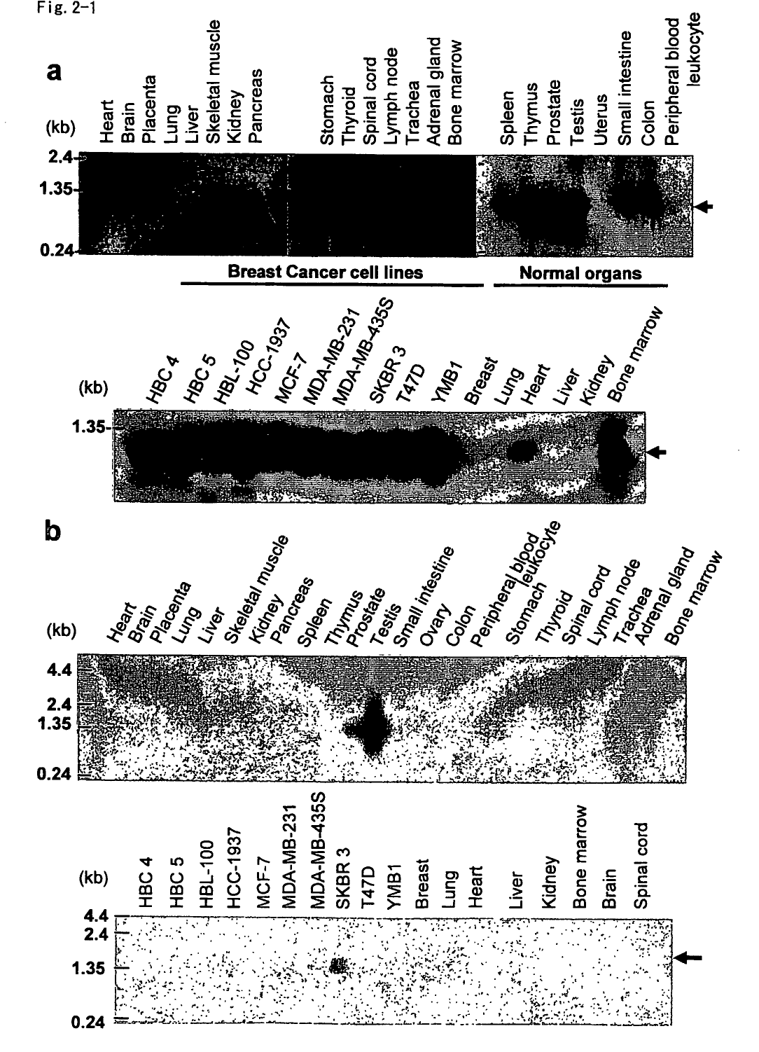

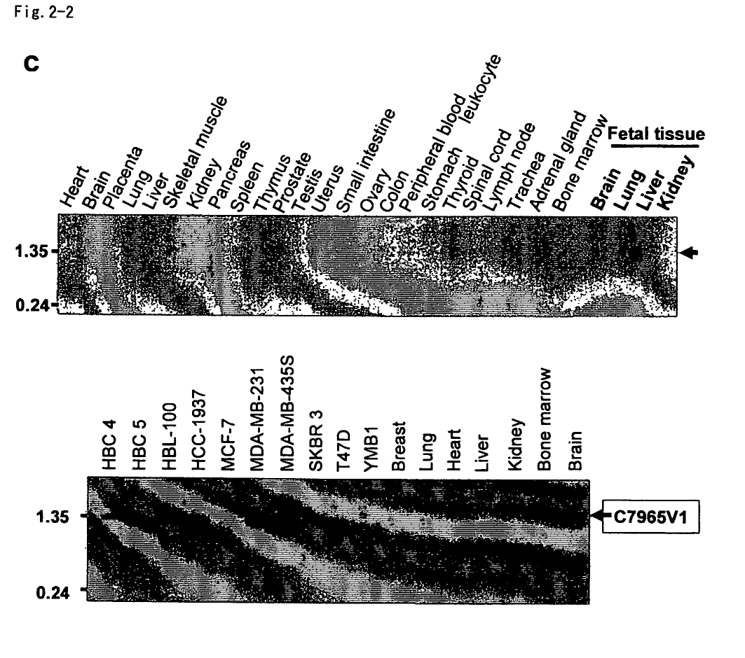

To further examine the expression pattern of these up-regulated genes, Northern blot analysis was performed with multiple-human tissues and breast cancer cell lines using cDNA fragments of A5657, B9769 and C7965 as probes (see above). As a result, A5657 was found to be ubiquitously expressed except lung, liver, pancreas and peripheral blood leukocytes (FIG. 2a; the upper panel), while was surprisingly over-expressed in all of breast cancer cell lines as compared to other normal tissues, especially heart and bone marrow, each of which showed strong signals as shown in the upper panel of FIG. 2a (FIG. 2a; the bottom panel). B9769 was exclusively expressed in testis and prostate (FIG. 2b, the upper panel) and was found to be over-expressed in some of breast cancer cell lines as compared to other normal tissues, especially normal human breast (FIG. 2b, the bottom panel). When Northern blot analysis was performed with multiple-tissues and breast cancer cell lines using a C7965 fragment within exon2 and 3 as a probe, approximately 1.35 kb transcripts were observed. The transcript of 1.35 kb was specifically expressed in breast cancer cell lines than in normal tissues including breast tissue (FIG. 2c, the bottom panel), and only weakly expressed in testis, skeletal muscle and small intestine (FIG. 2c, the upper panel).

Genomic Structure of A5657, B9769 and C7965:

To obtain the entire cDNA sequences for A5657, B9769 and C7965, RT-PCR was performed using a cDNA prepared from a breast cancer cell line and T47D as template. A5657 consists of 7 exons, designated HSPC150 protein similar to ubiquitin-conjugating enzyme, located on the chromosome 1q32.1 spanning approximately 10.3 kb in the genome. The full-length cDNA sequence of A5657 contained 928 nucleotides. The open reading frame (ORF) start at exon 2, and ends at exon 7. Eventually, this transcript encodes 197 amino acids.

B9769 consists of 8 exons, located on the chromosome 2q21.2 spanning approximately 5.7 kb in the genome. The full-length cDNA sequence of B9769 contained 1472 nucleotides. The ORF start at exon 1, and the ends at exon 8. Eventually, this transcript encodes 378 amino acids.

To further isolate the 5′ end of the C7965 transcript, rapid amplification of cDNA ends (RACE) was performed using a cDNA prepared from a breast cancer cell line, MCF7, as a template (see material and methods). A transcript was isolated consisting of 8 exons, corresponding to 1.35 kb in Northern blot (FIG. 2c, the bottom panel), respectively, and located on the chromosome 9q spanning approximately 28.8 kb in the genome. The full-length cDNA sequence of C7965 contained 1315 nucleotides. The ORF of the C7965 cDNA start within exon 1 and ends at exon 8. Eventually, this transcript encodes 288 amino acids.

Subcellular Localization of A5657. B9769 and C7965:

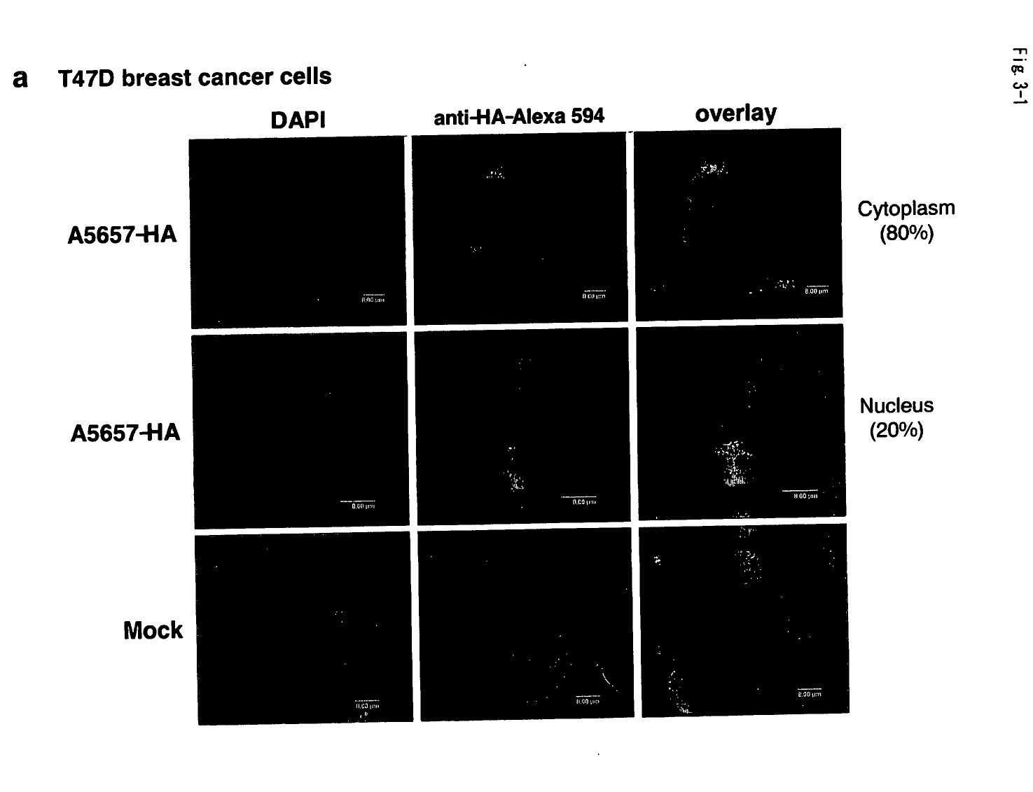

To further examine the characterization of A5657, B9769 and C7965, the sub-cellular localization of these gene products was examined in mammalian cells. Firstly, when we transiently transfected plasmids expressing the A5657 protein (pCAGGS-A5657-HA) were transiently transfected into T47D cells, immunocytochemical staining revealed exogenous A5657 localized throughout the cytoplasm in approximately 80% of all transfected-T47D cells and nucleus in the remaining approximately 20% of cells (FIG. 3a).

Next, when a plasmid expressing the B9769 protein (pCAGGS-Flag-B9769-HA) was transiently transfected into COS7 cells, immunocytochemical staining revealed that the B9769 protein was localized to the cytoplasmic apparatus as the intermediate filaments in a small number of cells (low density), but localized to the cytoplasm in a large number of cells (high density) (FIG. 3b), suggesting that B9769 may play a key role of interaction of cell to cell. To further examine the localization of this protein in more detail, localization of other cytoskeletal proteins was compared by immunocytochemical staining. As a result, B9769 and other cytoskeletal proteins were not co-localized to the cytoplasmic apparatus as the filaments in T47D cells (FIG. 3c).

Finally, a plasmid expressing C7965 protein (pcDNA3.1 (+)-C7965-Myc/H is) was transiently transfected into COS7 cells, immunocytochemical staining reveals the C7965 protein localized to the cytoplasmic apparatus in COS7 cells (FIG. 3d).

Growth-Inhibitory Effects of Small-Interfering RNA (siRNA) Designed to Reduce Expression of A5657, B9769 and C7965:

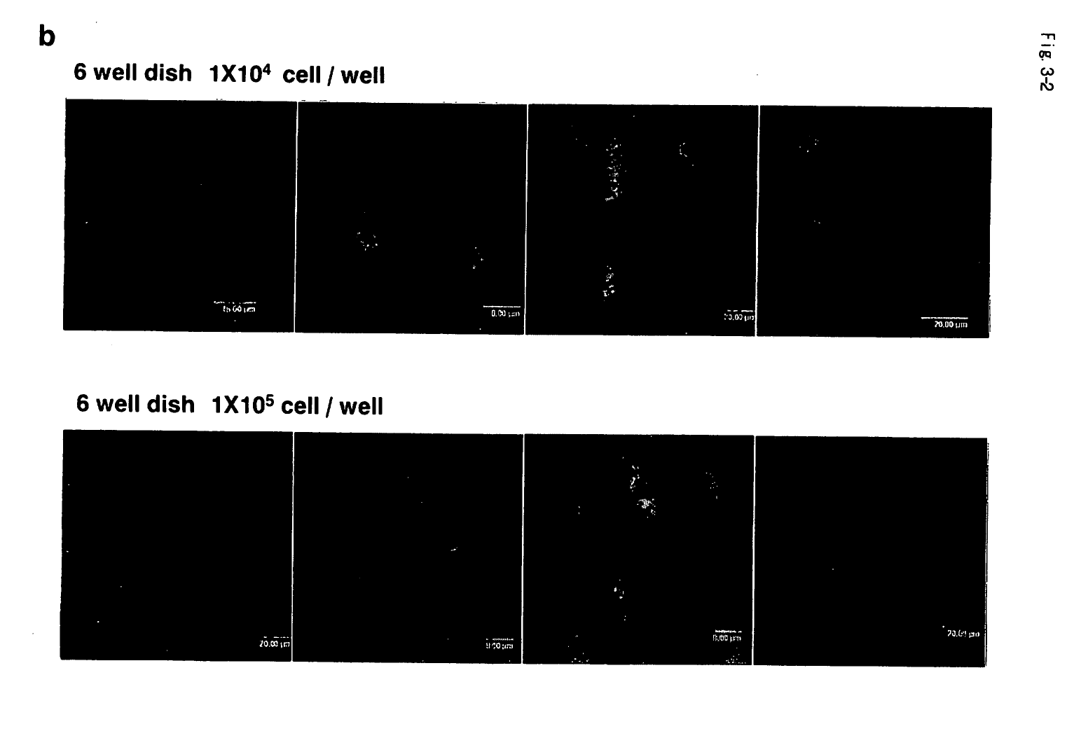

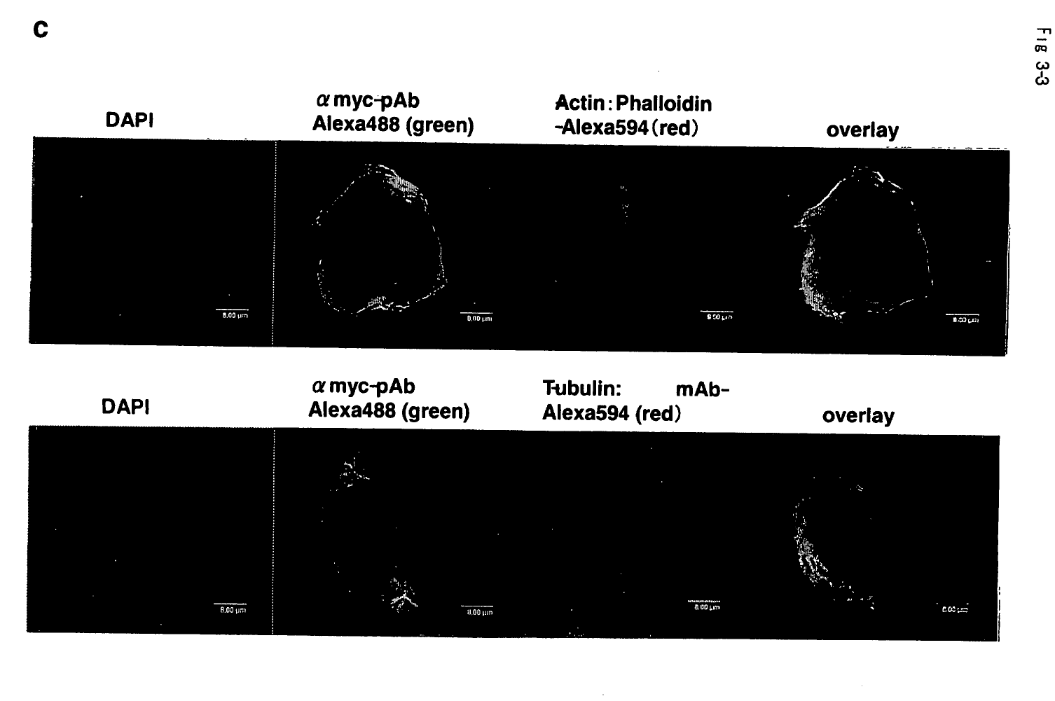

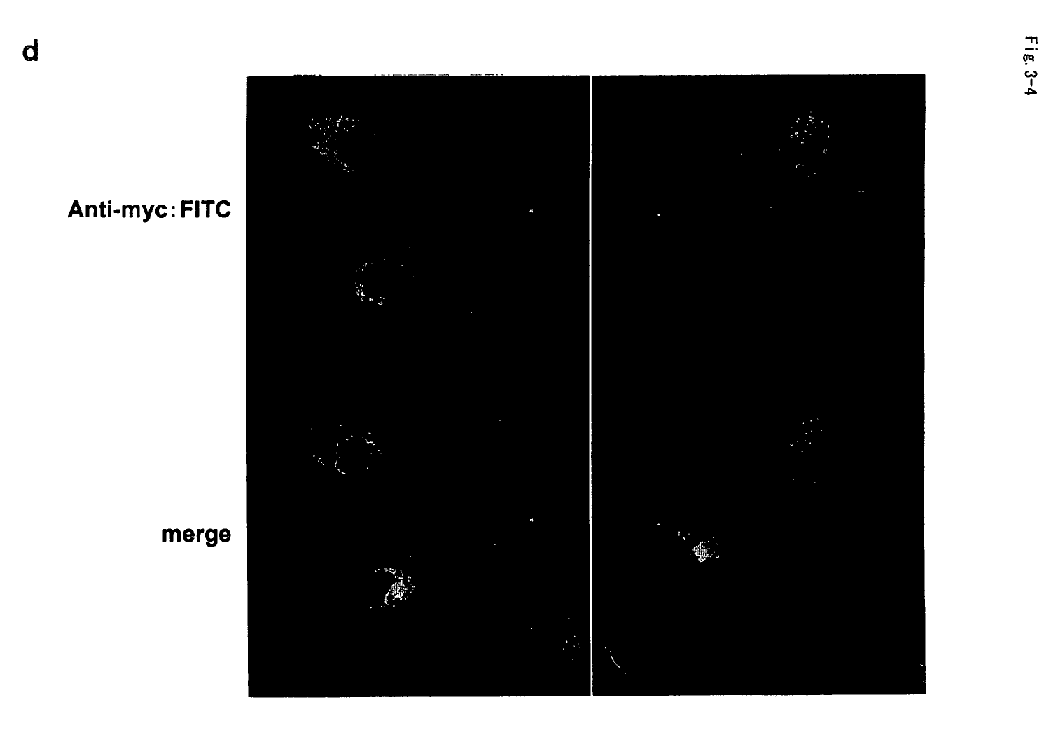

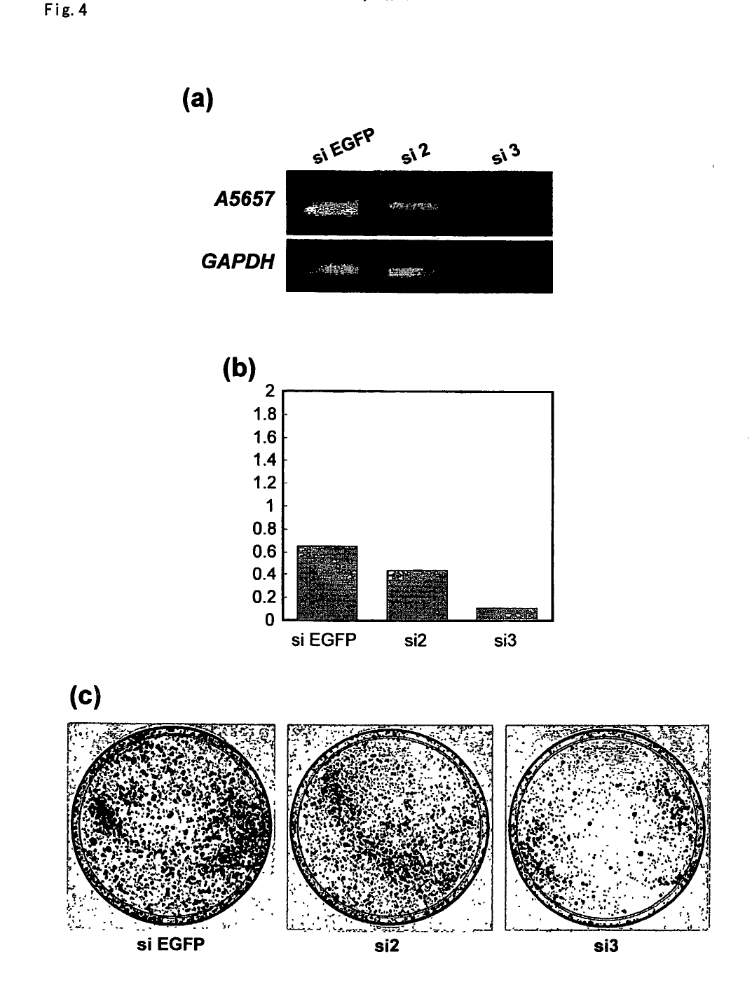

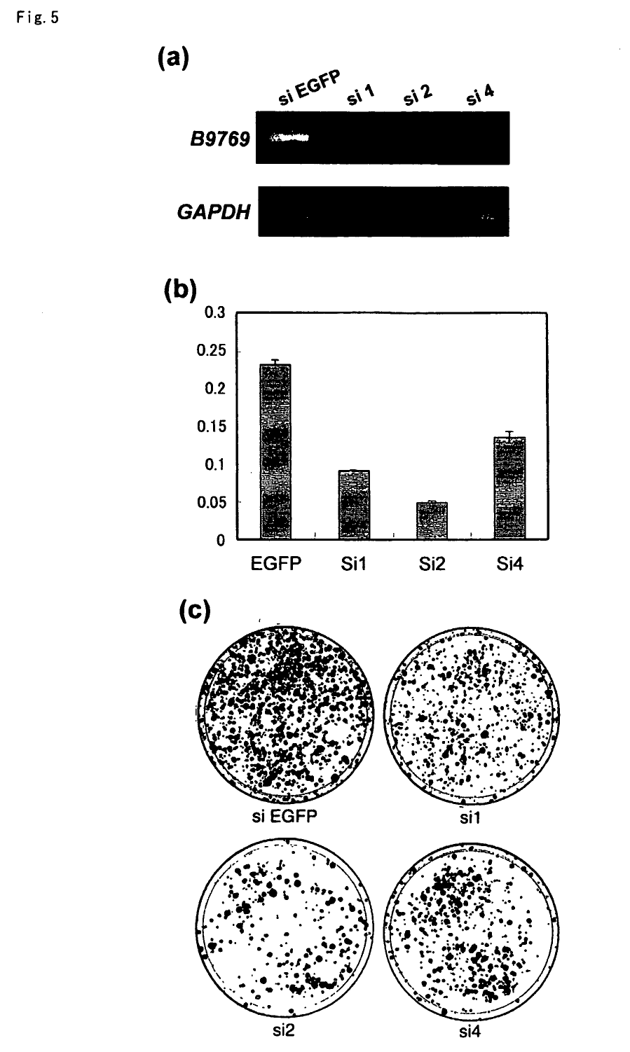

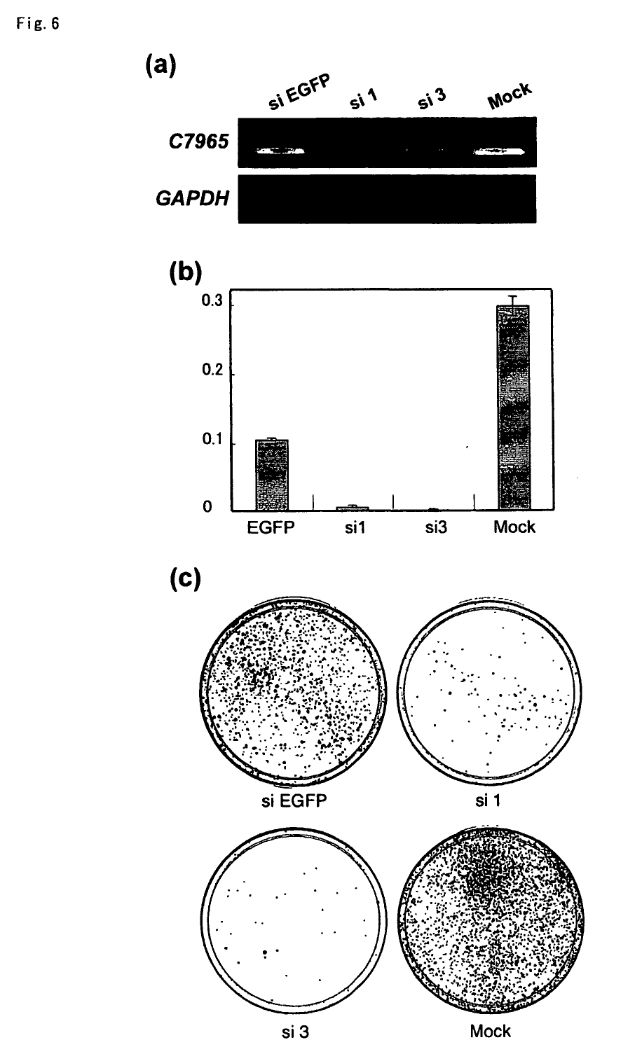

To assess the growth-promoting role of A5657, B9769 and C7965, the expression of endogenous A5657 and B9769 in the breast cancer line T47D, a line that has shown the over-expression of A5657, was knock down by means of the mammalian vector-based RNA interference (RNAi) technique (see Materials and Methods) (FIGS. 4, 5 and 6). Expression levels of A5657, B9769 and C7965 were examined by semi-quantitative RT-PCR experiments. As shown in FIGS. 4, 5 and 6, A5657 (si2 and si3), B9769 (si1 and si2, si4), and C7965 (si1 and si3)-specific siRNAs significantly suppressed expression of each gene compared with control siRNA construct (psiU6BX-EGFP). To confirm the cell growth inhibition with A5657, B9769 and C7965-specific siRNAs, colony-formation and MTT assays were performed, respectively. As a result, introduction of A5657 (si2 and si3) (FIG. 4b,c), B9769 (si1 and si2, si4) (FIG. 5b,c) and C7965 (si1 and si3) (FIG. 6b,c) constructs suppressed growth of T47D cells, consistent to the result of above reduced expression. Each result was verified by three independent experiments. Thus, these findings suggest that A5657, B9769 and C7965 have a significant function in the cell growth of the breast cancer.

Growth Promoting Effect of Transient Over-Expressing A5657. B9769 and C7965 into NIH3T3 Cells:

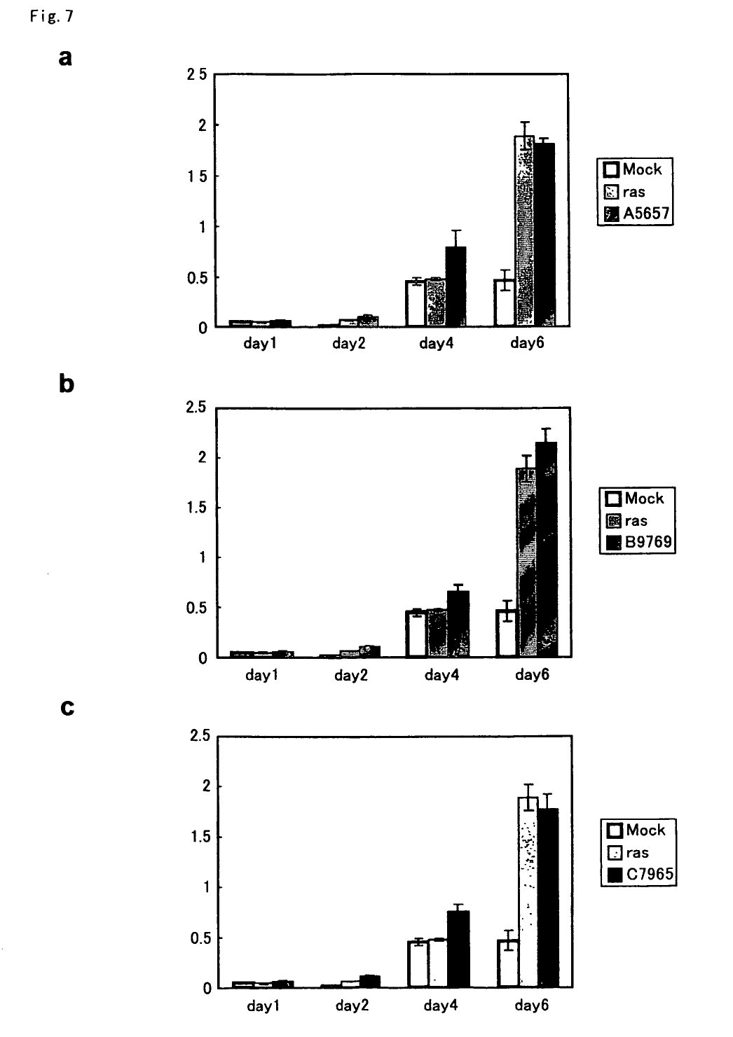

To further examine a possible role of A5657, B9769 and C7965 in the cell growth regulation, plasmids expressing A5657, B9769, C7965, H-ras mutant as positive control or Mock as negative control were transiently transfected into NIH3T3 cells, respectively, and then performed MTT assay (FIG. 7). After 6 days of transfection, over-expression of A5657 (FIG. 7a), B9769 (FIG. 7b) and C7965 (FIG. 7c) produced significant enhancement of cell growth (over 3-fold) as well as over-expression of H-ras mutant compared with NIH3T3 cells transfected with control plasmid (mock vector), suggesting that A5657, B9769 and C7965 might play roles in proliferation of cell growth. These results were confirmed by two independent experiments.

A5657 Protein Binds Ubiquitin:

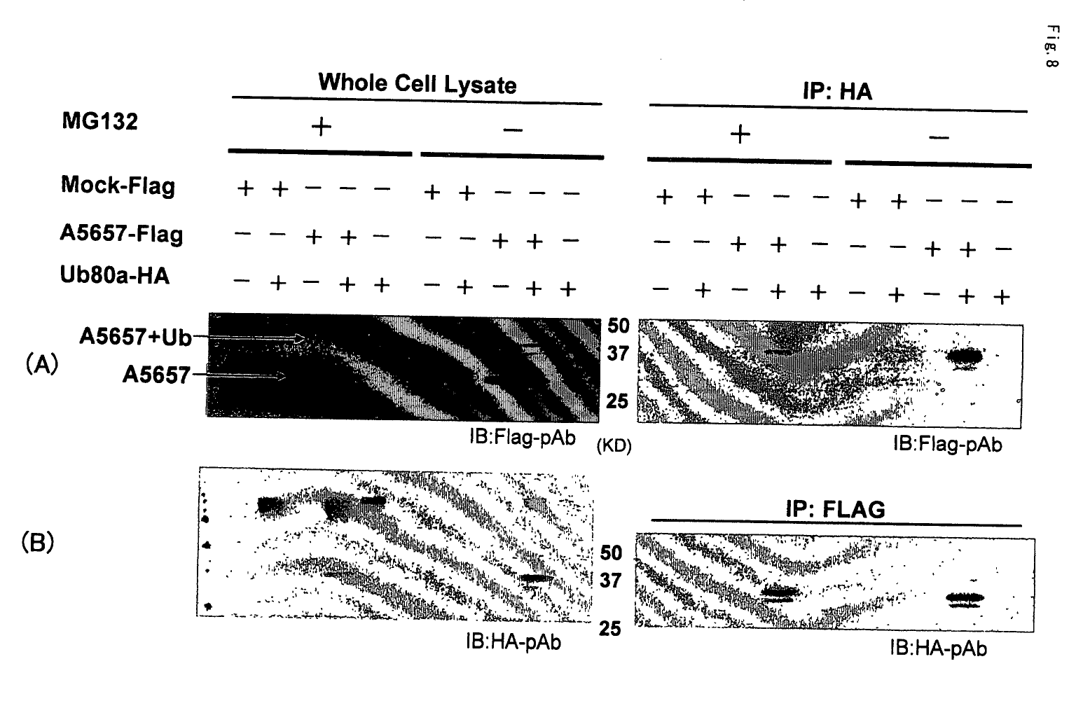

To explore the function of A5657 in more detail, Western blot analysis using whole cell lysates from A5657 expressing plasmid-transfected COS7 cells was performed. As a result, only one extra slower migrating band was observed in addition to the band showing the expected molecular weight (FIG. 8; left panel, in whole cell lysate), suggesting this might be generated by posttranslational modification. SMART program predicted A5657, encoded 197 amino-acid, would contain UBCc domain (Ubiquitin-conjugating enzyme E2, catalytic domain homologues) (5-152 residue), suggesting that A5657 might have a potential E2 ubiquitin enzyme activity. To investigate whether this extra band was due to mono-ubiquitination, plasmid DNAs designed to express FLAG-tagged A5657 (A5657-FLAG) and HA-tagged ubiquitin (UB80a-HA) were co-transfected into COS7 cells. A5657 was immunoprecipitated using anti-FLAG antibody and the precipitate was detectable with anti-HA antibody. An extra slow migrating band was observed, indicating that A5657 binds an ubiquitin (FIG. 8). Moreover, when immunopresipitation was performed using anti-HA antibody and then Western blot analysis with anti-FLAG, an extra slow migrating band was also observed as well (FIG. 8). Treatment of MG132, proteaseome inhibitor, had no effect on this binding. This finding strongly suggests that A5657 protein might have E2 ubiquitin enzyme activity via mono-ubiquitination.

Discussion

Herein, through the precise expression profiles of breast cancer by means of genome wide cDNA microarray, novel genes, A5657, B9769 and C7965, were isolated that were significantly over-expressed in breast cancer cells as compared to normal human tissues.

Among them, AS657 contains a UBCc domain (ubiquitin-conjugating enzyme E2, catalytic domain homologues) and, accordingly, may bind ubiquitin. This finding strongly suggests that the A5657 protein has E2 ubiquitin enzyme activity via mono-ubiquitination, and, accordingly, may be involved in breast cancer tumorigenesis.

The B9769 protein was observed by immunochemical staining to localize in cytoplasm as intermediate filaments. The B9769 protein was further observed to localize in the cytoplasmic apparatus as the intermediate filaments under conditions of low density cell population, but in the cytoplasm under conditions of high density cell population, which suggests that B9769 may play a key role in cell to cell interaction.

As demonstrated herein, treatment of breast cancer cells with siRNA effectively inhibited expression of all three target genes, A5657, B9769 and C7965, which, in turn, significantly suppressed breast cancer cell-tumor growth. Moreover, these genes when transiently over-expressed in NIH3T3 cells dramatically were demonstrated to promote cell proliferation in MTT assay. These findings suggest that A5657, B9769 and C7965 play key roles in tumor cell growth proliferation and are promising targets for development of anti-cancer drugs.

TABLE 2
Histoclinical information

| | age in | memopause | | | | | Histrogical | Lymphocytic | | | |
|---|---|---|---|---|---|---|---|---|---|---|---|
| ID | operation | status | T | N | M | Stage | type | infiltrate | Angioinvasion | ER | PgR |
| MMK010003 | 51 | pre | 2 | 1 | 0 | 2 | a3 | 3 | 0 | + | + |
| MMK010004 | 47 | pre | 2 | 1 | 0 | 2 | a1 | 0 | 0 | + | + |
| MMK010005 | 44 | pre | 2 | 0 | 0 | 2 | a1 | 1 | 0 | + | + |
| MMK010013 | 45 | pre | 2 | 1 | 0 | 2 | a1 | 1 | 0 | − | − |
| MMK010016 | 44 | pre | 2 | 0 | 0 | 2 | a2 | 0 | 0 | − | − |
| MMK010025 | 46 | pre | 2 | 0 | 0 | 2 | a1 | 0 | 0 | + | + |
| MMK010031 | 29 | pre | 2 | 2 | 0 | 3 | a3 | 3 | 0 | − | − |
| MMK010037 | 62 | post | 0 | 0 | 0 | 0 | Ia | 0 | 0 | + | + |
| MMK010042 | 47 | pre | 2 | 1 | 0 | 2 | a3 | 1 | 2 | + | + |
| MMK010086 | 42 | pre | 2 | 0 | 0 | 2 | a1 | 0 | 0 | + | + |
| MMK010102 | 51 | pre | 2 | 1 | 0 | 3 | a2 | 3 | 0 | + | + |
| MMK010110 | 39 | pre | 2 | 0 | 0 | 2 | a1 | 2 | 0 | − | − |
| MMK010129 | 52 | pre | 2 | 2 | 0 | 3 | a1 | 2 | 0 | − | − |
| MMK010135 | 41 | pre | 2 | 0 | 0 | 2 | a1 | 0 | 0 | + | + |
| MMK010138 | 38 | pre | 2 | 0 | 0 | 2 | a1 | 0 | 0 | + | + |
| MMK010145 | 51 | pre | 2 | 1 | 0 | 2 | a3 | 0 | 0 | + | + |
| MMK010147 | 49 | pre | 2 | 1 | 0 | 2 | a1 | 1 | 0 | + | + |
| MMK010149 | 35 | pre | 2 | 0 | 0 | 2 | a3 | 1 | 0 | − | − |
| MMK010175 | 38 | pre | 2 | 0 | 0 | 2 | a3 | 0 | 0 | + | + |
| MMK010178 | 51 | pre | 0 | 0 | 0 | 0 | Ia | 0 | 0 | + | + |
| MMK010207 | 40 | pre | 2 | 0 | 0 | 2 | a1 | 0 | 0 | + | + |
| MMK010214 | 42 | pre | 2 | 1 | 0 | 2 | a1 | 0 | 0 | − | − |
| MMK010247 | 48 | pre | 2 | 1 | 0 | 2 | a2 | 3 | 0 | − | − |
| MMK010252 | 52 | pre | 2 | 1 | 0 | 2 | a2 | 0 | 0 | − | − |
| MMK010255 | 47 | pre | 2 | 0 | 0 | 2 | a2 | 0 | 0 | − | − |
| MMK010302 | 46 | pre | 2 | 1 | 0 | 2 | a2 | 2 | 1 | − | − |
| MMK010304 | 48 | pre | 2 | 1 | 0 | 2 | a3 | 1 | 0 | + | + |
| MMK010326 | 53 | post | 0 | 0 | 0 | 0 | Ia | 0 | 0 | − | − |
| MMK010327 | 43 | pre | 2 | 1 | 0 | 2 | a1 | 1 | 1 | + | + |
| MMK010341 | 42 | pre | 2 | 1 | 0 | 2 | a1 | 2 | 0 | + | + |
| MMK010370 | 46 | pre | 2 | 1 | 0 | 2 | a3 | 2 | 0 | + | + |
| MMK010397 | 38 | pre | 2 | 1 | 0 | 2 | a3 | 3 | 2 | + | + |
| MMK010411 | 46 | pre | 2 | 0 | 0 | 2 | a1 | 0 | 0 | + | + |
| MMK010431 | 50 | pre | 2 | 0 | 0 | 2 | a3 | 0 | 0 | − | − |
| MMK010435 | 49 | pre | 2 | 1 | 0 | 2 | a3 | 0 | 0 | + | + |
| MMK010453 | 49 | pre | 2 | 1 | 0 | 2 | a3 | 3 | 0 | + | + |
| MMK010471 | 42 | pre | 2 | 1 | 0 | 2 | a1 | 3 | 0 | − | − |
| MMK010473 | 40 | pre | 2 | 1 | 0 | 2 | a2 | 0 | 0 | − | − |
| MMK010478 | 38 | pre | 2 | 2 | 0 | 3 | a2 | 0 | 0 | + | + |
| MMK010491 | 46 | pre | 2 | 0 | 0 | 2 | a3 | 1 | 0 | + | + |
| MMK010497 | 44 | pre | 0 | 0 | 0 | 0 | Ia | 0 | 0 | − | + |
| MMK010500 | 45 | pre | 2 | 0 | 0 | 2 | a1 | 0 | 0 | + | + |
| MMK010502 | 51 | pre | 2 | 0 | 0 | 2 | a2 | 0 | 0 | − | − |
| MMK010508 | 51 | pre | 2 | 1 | 0 | 2 | a2 | 0 | 0 | − | − |
| MMK010521 | 21 | pre | 2 | 0 | 0 | 2 | a1 | 1 | 1 | − | − |
| MMK010552 | 49 | pre | 2 | 0 | 0 | 2 | a2 | 0 | 0 | − | − |
| MMK010554 | 51 | pre | 2 | 0 | 0 | 2 | a3 | 2 | 0 | + | + |
| MMK010571 | 45 | pre | 2 | 1 | 1 | 4 | a3 | 3 | 0 | + | + |
| MMK010591 | 40 | pre | 0 | 0 | 0 | 0 | Ia | 0 | 0 | − | + |
| MMK010613 | 37 | pre | 0 | 0 | 0 | 0 | Ia | 0 | 0 | − | + |
| MMK010623 | 39 | pre | 2 | 1 | 0 | 2 | a1 | 3 | 0 | + | + |
| MMK010624 | 39 | pre | 2 | 1 | 0 | 2 | a1 | 3 | 0 | + | + |
| MMK010626 | 48 | pre | 2 | 0 | 0 | 2 | a1 | 1 | 1 | − | − |
| MMK010631 | 41 | pre | 2 | 0 | 0 | 2 | a1 | 0 | 0 | + | + |
| MMK010640 | 35 | pre | 0 | 0 | 0 | 0 | Ia | 0 | 0 | + | + |
| MMK010644 | 47 | pre | 2 | 2 | 0 | 2 | a3 | 3 | 0 | + | + |
| MMK010646 | 37 | pre | 2 | 1 | 0 | 2 | a3 | 1 | 0 | + | + |
| MMK010660 | 46 | pre | 2 | 0 | 0 | 2 | a1 | 0 | 0 | − | − |
| MMK010671 | 45 | pre | 2 | 0 | 0 | 2 | a1 | 0 | 0 | − | − |
| MMK010679 | 68 | post | 0 | 0 | 0 | 0 | Ia | 0 | 0 | + | + |
| MMK010680 | 58 | post | 0 | 0 | 0 | 0 | Ia | 0 | 0 | − | + |
| MMK010709 | 33 | pre | 2 | 0 | 0 | 2 | a3 | 0 | 2 | − | − |
| MMK010711 | 51 | pre | 0 | 0 | 0 | 0 | Ia | 0 | 0 | − | + |
| MMK010724 | 40 | pre | 2 | 1 | 0 | 2 | a3 | 3 | 2 | + | + |
| MMK010744 | 41 | pre | 0 | 0 | 0 | 0 | Ia | 0 | 0 | + | + |
| MMK010758 | 40 | pre | 2 | 1 | 0 | 2 | a1 | 0 | 1 | + | + |
| MMK010760 | 42 | pre | 2 | 0 | 0 | 2 | a1 | 0 | 0 | + | + |
| MMK010762 | 50 | pre | 2 | 1 | 0 | 2 | a3 | 3 | 1 | + | + |
| MMK010769 | 33 | pre | 2 | 0 | 0 | 2 | a2 | 0 | 0 | − | − |
| MMK010772 | 45 | pre | 2 | 1 | 0 | 2 | a3 | 2 | 0 | − | − |
| MMK010779 | 46 | pre | 2 | 1 | 0 | 2 | a2 | 0 | 1 | − | − |
| MMK010780 | 31 | pre | 2 | 0 | 0 | 2 | a2 | 0 | 0 | − | − |
| MMK010781 | 44 | pre | 2 | 0 | 0 | 2 | a3 | 0 | 2 | + | + |
| MMK010794 | 52 | pre | 2 | 1 | 0 | 2 | a3 | 2 | 1 | + | + |
| MMK010818 | 51 | pre | 2 | 0 | 0 | 2 | a1 | 0 | 2 | + | + |
| MMK010835 | 42 | pre | 0 | 0 | 0 | 0 | Ia | 0 | 0 | + | + |
| MMK010846 | 47 | pre | 2 | 0 | 0 | 2 | a1 | 0 | 0 | + | + |
| MMK010858 | 42 | pre | 2 | 1 | 0 | 2 | a3 | 2 | 3 | + | + |
| MMK010864 | 52 | pre | 2 | 1 | 0 | 2 | a1 | 0 | 1 | − | − |
| MMK010869 | 45 | pre | 2 | 0 | 0 | 2 | a1 | 0 | 1 | − | − |
| MMK010903 | 47 | pre | 2 | 0 | 0 | 2 | a1 | 0 | 0 | + | + |

### INDUSTRIAL APPLICABILITY

The gene-expression analysis of breast cancer described herein, obtained through a combination of laser-capture dissection and genome-wide cDNA microarray, has identified specific genes as targets for cancer prevention and therapy. Based on the expression of a subset of these differentially expressed genes, the present invention provides molecular diagnostic markers for identifying or detecting breast cancer.

The methods described herein are also useful in the identification of additional molecular targets for prevention, diagnosis and treatment of breast cancer. The data reported herein add to a comprehensive understanding of breast cancer, facilitate development of novel diagnostic strategies, and provide clues for identification of molecular targets for therapeutic drugs and preventative agents. Such information contributes to a more profound understanding of breast tumorigenesis, and provide indicators for developing novel strategies for diagnosis, treatment, and ultimately prevention of breast cancer.

All patents, patent applications, and publications cited herein are incorporated by reference in their entirety.

Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method of diagnosing breast cancer or a predisposition to developing breast cancer in a subject, comprising determining a level of expression of a breast cancer-associated gene in a patient-derived biological sample selected from the group consisting of A5657, B9769, and C7965, wherein an increase in said sample expression level as compared to a normal control level of said gene indicates that said subject suffers from or is at risk of developing breast cancer.

2. The method of claim 1, wherein said sample expression level is at least 10% greater than said normal control level.

3. The method of claim 1, wherein said breast cancer-associated gene is the A5657 gene, further wherein an increase in said sample expression level as compared to a normal control level indicates said subject suffers from or is at risk of developing IDC.

4. The method of claim 3, wherein said sample expression level is at least 10% greater than said normal control level.

5. The method of claim 1, wherein said method further comprises determining the level of expression of a plurality of said breast cancer-associated genes.

6. The method of claim 1, wherein gene expression level is determined by a method selected from the group consisting of:
(a) detecting mRNA of a breast cancer-associated gene selected from the group consisting of A5657, B9769, and C7965,
(b) detecting a protein encoded by a breast cancer-associated gene selected from the group consisting of A5657, B9769, and C7965, and
(c) detecting a biological activity of a protein encoded by a breast cancer-associated gene selected from the group consisting of A5657, B9769, and C7965.

7. The method of claim 6, wherein said detection is carried out on a DNA array.

8. The method of claim 1, wherein said patient-derived biological sample comprises a breast tissue.

9. The method of claim 8, wherein said breast tissue comprises an epithelial cell.

10. The method of claim 1, wherein said patient-derived biological sample comprises a breast cancer cell.

11. The method of claim 1, wherein said patient-derived biological sample comprises an epithelial cell from a breast cancer cell.

12. A method of screening for a compound for treating or preventing breast cancer, said method comprising the steps of:
a) contacting a test compound with a polypeptide encoded by a polynucleotide selected from the group consisting of A5657, B9769, and C7965;
b) detecting the binding activity between the polypeptide and the test compound; and
c) selecting the test compound that binds to the polypeptide.

13. A method of screening for a compound for treating or preventing breast cancer, said method comprising the steps of:
a) contacting a candidate compound with a cell expressing one or more marker genes, wherein the one or more marker genes is selected from the group consisting of A5657, B9769, and C7965; and
b) selecting the candidate compound that reduces the expression level of said one or more marker as compared to a control.

14. The method of claim 13, wherein said cell comprises a breast cancer cell.

15. A method of screening for a compound for treating or preventing breast cancer, said method comprising the steps of:
a) contacting a test compound with a polypeptide encoded by a polynucleotide selected from the group consisting of A5657, B9769, and C7965;
b) detecting the biological activity of the polypeptide of step (a); and
c) selecting the test compound that suppresses the biological activity of said polypeptide as compared the biological activity of said polypeptide detected in the absence of the test compound.

16. A method of screening for compound for treating or preventing breast cancer, said method comprising the steps of:
a) contacting a candidate compound with a cell into which a vector, comprising the transcriptional regulatory region of one or more marker genes selected from the group consisting of A5657, B9769, and C7965 and a reporter gene that is expressed under the control of the transcriptional regulatory region, has been introduced;
b) measuring the expression level or activity of said reporter gene; and
c) selecting the candidate compound that reduces the expression level or activity of said reporter gene as compared to a control.

17. The method of claim 12, wherein said breast cancer is IDC, said method comprises the steps of:
a) contacting a test compound with a polypeptide encoded by A5657;
b) detecting the binding activity between the polypeptide and the test compound; and
c) selecting the test compound that binds to the polypeptide.

18. The method of claim 13, wherein said breast cancer is IDC and said method comprises the steps of:
a) contacting a candidate compound with a cell expressing A5657; and
b) selecting the candidate compound that reduces the expression level of A5657, as compared to a control.

19. The method of claim 18, wherein said cell comprises an IDC cell.

20. The method of claim 15, wherein said breast cancer is IDC and said method comprises the steps of:
a) contacting a test compound with a polypeptide encoded by A5657;
b) detecting the biological activity of the polypeptide of step (a); and
c) selecting the test compound that suppresses the biological activity of said polypeptide as compared to the biological activity of said polypeptide detected in the absence of the test compound.

21. A method of claim 16, wherein said breast cancer is IDC and said method comprises the steps of:
a) contacting a candidate compound with a cell into which a vector, comprising the transcriptional regulatory region of A5657 and a reporter gene that is expressed under the control of the transcriptional regulatory region, has been introduced;
b) measuring the expression level or activity of said reporter gene; and
c) selecting the candidate compound that reduces the expression level or activity of said reporter gene as compared to a control.

22. A kit comprising a detection reagent which binds to two or more nucleic acid sequences selected from the group consisting of A5657, B9769, and C7965, or polypeptides encoded thereby.

23. A method of treating or preventing breast cancer in a subject comprising administering to said subject an antisense composition, said antisense composition comprising a nucleotide sequence complementary to a coding sequence corresponding to a gene selected from the group consisting of A5657, B9769, and C7965.

24. A method of treating or preventing breast cancer in a subject comprising administering to said subject an siRNA composition, wherein said siRNA composition reduces the expression of a nucleic acid sequence selected from the group consisting of A5657, B9769, and C7965.

25. The method of claim 24, wherein said siRNA comprises the sense strand comprising a nucleotide sequence selected from the group consisting of nucleotide sequences of SEQ ID NO: 28, 29, 30, 31, 32, 33, and 34.

26. A method for treating or preventing breast cancer in a subject comprising the step of administering to said subject a pharmaceutically effective amount of an antibody or immunologically active fragment thereof that binds to a protein encoded by any one gene selected from the group consisting of A5657, B9769, and C7965.

27. A method of treating or preventing breast cancer in a subject comprising administering to said subject a vaccine comprising a polypeptide encoded by a nucleic acid selected from the group consisting of A5657, B9769, and C7965 or an immunologically active fragment of said polypeptide, or a polynucleotide encoding the polypeptide.

28. A method for inducing anti-tumor immunity, said method comprising the step of contacting with an antigen presenting cell a polypeptide, a polynucleotide encoding said polypeptide, or a vector comprising the said polynucleotide, wherein the polypeptide is encoded by a gene selected from the group consisting of A5657, B9769, and C7965, or a immunologically active fragment thereof.

29. The method for inducing anti-tumor immunity of claim 27, wherein the method further comprises the step of administering the antigen presenting cell to a subject.

30. A method for treating or preventing breast cancer in a subject, said method comprising the step of administering a compound obtained by the method according to any one of claims 12-21.

31. The method of claim 23, wherein said breast cancer is IDC and said antisense composition comprises a nucleotide sequence complementary to a coding sequence corresponding to A5657.

32. The method of claim 24, wherein said breast cancer is IDC and said siRNA composition reduces the expression A5657.

33. The method of claim 32, wherein said siRNA comprises the sense strand comprising a nucleotide sequence of SEQ ID NO: 28 or 29.

34. The method of claim 26, wherein said breast cancer is IDC and said antibody or fragment thereof binds to a protein encoded by A5657.

35. The method of claim 27, wherein said breast cancer is IDC and said vaccine comprises a polypeptide encoded by A5657, or an immunologically active fragment of said polypeptide, or a polynucleotide encoding said polypeptide.

36. A method for treating or preventing breast cancer in a subject, wherein said breast cancer is IDC and wherein said method comprises the step of administering a compound obtained by the method according to any one of claims 17-21.

37. A composition for treating or preventing breast cancer, said composition comprising a pharmaceutically effective amount of an antisense polynucleotide or small interfering RNA against a polynucleotide selected from the group consisting of A5657, B9769, and C7965.

38. The composition of claim 37, wherein said siRNA comprises the sense strand comprising a nucleotide sequence selected from the group consisting of nucleotide sequences of SEQ ID NO: 28, 29, 30, 31, 32, 33, and 34.

39. A composition for treating or preventing breast cancer, said composition comprising a pharmaceutically effective amount of an antibody or fragment thereof that binds to a protein encoded by a gene selected from the group consisting of A5657, B9769, and C7965.

40. A composition for treating or preventing breast cancer, said composition comprising as an active ingredient a pharmaceutically effective amount of a compound selected by the method of any one of claims 12-16, and a pharmaceutically acceptable carrier.

41. The composition of claim 37, wherein said breast cancer is IDC and said polynucleotide is A5657.

42. The composition of claim 41, wherein said siRNA comprises the sense strand comprising a nucleotide sequence of SEQ ID NO: 28 or 29.

43. The composition of claim 39, wherein said breast cancer is IDC and said protein is encoded by A5657.

44. A composition for treating or preventing breast cancer, wherein said breast cancer is IDC and wherein said composition comprises as an active ingredient a pharmaceutically effective amount of a compound selected by the method of any one of claims 17-21, and a pharmaceutically acceptable carrier.
